(19) United States
(12) Patent Application Publication
(10) Pub. No.: US 2009/0018163 A1
(43) Pub. Date: Jan. 15, 2009

(54) Substituted Heterocyclic Ethers and Their Use in CNS Disorders

(75) Inventors: William D. Schmitz, Cheshire, CT (US); Irene E. Whitney, Lebanon, NH (US); Andrew P. Degnan, Rocky Hill, CT (US); Joanne J. Bronson, Durham, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/169,874

(22) Filed: Jul. 9, 2008

Related U.S. Application Data
(60) Provisional application No. 60/949,018, filed on Jul. 11, 2007.

(51) Int. Cl. A61K 31/4709; C07D 401/12; A61P 25/00

(52) U.S. Cl. 514/314; 546/152

(57) ABSTRACT

The invention encompasses compounds of Formula I, including pharmaceutically acceptable salts, their pharmaceutical compositions, and their use in treating CNS disorders.

### CROSS REFERENCE TO RELATED APPLICATIONS

This application claims the benefit of U.S. provisional application No. 60/949,018 filed Jul. 11, 2007.

### BACKGROUND OF THE INVENTION

Tachykinins are a group of naturally occurring peptides found widely distributed throughout mammals, both within the central nervous system and in the peripheral nervous and circulatory systems. The three known mammalian tachykinins are Neurokinin-1 (NK-1, substance P), Neurokinin A, and Neurokinin B. These compounds act as neurotransmitters and immunomodulators and may contribute to the pathophysiology of a wide variety of human diseases.

Receptors for tachykinins have been identified and include neurokinin-1 (NK-1 or Substance P-preferring), NK-2 (Neurokinin A-preferring) and NK-3 (Neurokinin B-preferring). NK-1 receptor antagonists are being developed for the treatment of physiological conditions associated with an excess or imbalance of tachykinins, particularly substance P. Such conditions include affective disorders such as anxiety, depression, obsessive compulsive disorder, bulimia, and panic disorder. See Gentsch et al. Behav. Brain Res. 2002, 133, 363; Varty et al. Neuropsychopharmacology 2002, 27, 371; Papp et al. Behav. Brain Res. 2000, 115, 19; Kramer et al. Science 1998, 281, 1640; and Rosen et al. Bioorg. Med. Chem. Lett. 1998, 8, 281. Robust antidepressant activity has been reported for two NK-1 antagonists, MK-869 (M. S. Kramer, et al., Science 1998, 281, 1640) and CP-122,721 (T. J. Rosen, et al., Bioorganic and Medicinal Chemistry Letters 1998, 8, 28 and CNS Drug News, December, 2000, 24).

Selective serotonin reuptake inhibitors (SSRI's) have proven to be effective in treating depression, but have the disadvantages of delayed onset of antidepressant activity, limited efficacy, and significant side effects. See Novel strategies for pharmacotherapy of depression, K. A. Maubach, N. M. J. Rupniak, M. S. Kramer, and R. G. Hill, Current Opinion in Chemical Biology 1999, 3, 491-499. Selective serotonin reuptake inhibitors (SSRIs) in combination with other agents can be useful for the treatment of depression and other disorders and combination SERT/NK1 compounds should also be useful for these conditions. For example, the combination of SSRIs with dopamine reuptake inhibitors such bupropion and modafanil have shown clinical benefit relative to SSRIs alone, primarily due to superior side effect profiles (Bodkin et al, 1997, J Clin Psychiatry, 58: 137-145; Kennedy et al, 2002, J Clin Psychiatry, 63:181-186). Additionally, the combination of SSRIs with 5-HT1A antagonists such as pindolol have shown improved clinical response relative to SSRIs alone (Artigas F et al, 1994, Arch Gen Psychiatry 51:248-251; Blier P and Bergeron R, 1995, J Clin Psychopharmacol 15:217-222). Finally, combining SSRIs with antipsychotics, such as fluoxetine plus olanzapine, has provided superior profiles in certain depressed populations including psychotic depression and bipolar depression (Corya et al, 2003, J Clin Psychiatry, 64:1349-1356; Rothschild et al, 2004, J Clin Psychopharmacol, 24:365-373).

NK-1 antagonists are believed to modulate 5-HT function via noradrenergic pathways and have been shown to attenuate presynaptic 5-HT1A receptor function. NK-1 antagonists offer an alternative approach for treating depression in patients that respond poorly to the SSRI's and other available drugs and the combination of serotonin reuptake inhibition with NK-1 antagonism may lead to new classes of drugs with improved characteristics.

### DESCRIPTION OF THE INVENTION

The invention encompasses compounds of Formula I, including pharmaceutically acceptable salts, their pharmaceutical compositions, and their use in treating CNS disorders.

One aspect of the invention are compounds of Formula I

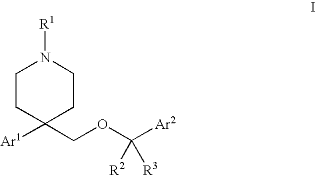

where:
R¹ is hydrogen or alkyl;
R² is hydrogen or alkyl
R³ is hydrogen or alkyl;
R⁴ is amino, alkylamino, dialkylamino, pyrrolidinyl, piperidinyl, piperazinyl, (alkyl)piperazinyl, morpholinyl, or thiomorpholinyl:
R⁵ is hydrogen or alkyl;
Ar¹ is phenyl or pyridinyl and is substituted with 0-3 substituents selected from the group consisting of halo, alkyl, haloalkyl, alkoxy, and cyano;
Ar² is quinolinyl, isoquinolinyl, quinazolinyl, or quinoxalinyl and is substituted with 0-3 substituents selected from the group consisting of halo, alkyl, cycloalkyl, haloalkyl, hydroxy, alkoxy, haloalkoxy, cyano, R⁴, COR⁴, CO₂R⁵, and Ar³; and
Ar³ is phenyl substituted with 0-3 substituents selected from the group consisting of halo, alkyl, haloalkyl, hydroxy, alkoxy, haloalkoxy, and cyano;
or a pharmaceutically acceptable salt thereof.

Another aspect of the invention are compounds of Formula I where R¹ is hydrogen.

Another aspect of the invention are compounds of Formula I where R¹ is methyl.

Another aspect of the invention are compounds of Formula I where R² and R³ are hydrogen.

Another aspect of the invention are compounds of Formula I where R² is methyl and R³ is hydrogen.

Another aspect of the invention are compounds of Formula I where Ar¹ is phenyl.

Another aspect of the invention are compounds of Formula I where Ar¹ is halophenyl.

Another aspect of the invention is a compound of formula I where Ar² is quinolinyl substituted with 0-3 substituents selected from the group consisting of halo, alkyl, haloalkyl, hydroxy, alkoxy, haloalkoxy, cyano, R⁴, COR⁴, CO₂R⁵, and Ar³.

Any scope of a substituent, including R¹, R², R³, R⁴, R⁵, Ar¹, Ar², and Ar³, can be used independently with the scope of any other instance of a substituent.

Unless specified otherwise, these terms have the following meanings. “Alkyl” means a straight or branched alkyl group composed of 1 to 6 carbons. “Alkenyl” means a straight or branched alkyl group composed of 2 to 6 carbons with at least one double bond. “Cycloalkyl” means a monocyclic ring system composed of 3 to 7 carbons. “Hydroxyalkyl,” “alkoxy” and other terms with a substituted alkyl moiety include straight and branched isomers composed of 1 to 6 carbon atoms for the alkyl moiety. “Haloalkyl” and “haloalkoxy” include all halogenated isomers from monohalo substituted alkyl to perhalo substituted alkyl. “Aryl” includes carbocyclic and heterocyclic aromatic substituents. Parenthetic and multiparenthetic terms are intended to clarify bonding relationships to those skilled in the art. For example, a term such as ((R)alkyl) means an alkyl substituent further substituted with the substituent R.

Heteroaryl compounds include tautomeric forms. Two examples include the following:

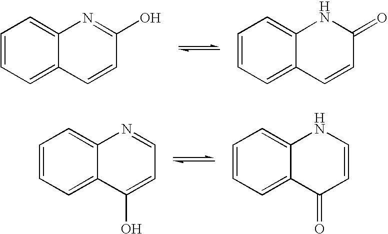

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some Formula I compounds contain at least one asymmetric carbon atom, an example of which is shown below. The invention includes all stereoisomeric forms of the compounds, both mixtures and separated isomers. Mixtures of stereoisomers can be separated into individual isomers by methods known in the art.

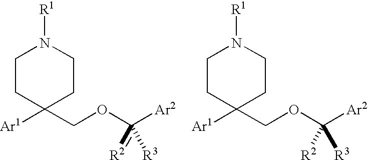

### Synthetic Methods

Compounds of Formula I can be made according to methods known in the art and those illustrated in the schemes below and in the specific embodiments section. The compounds can be made by reasonable variations known in the art. The variables describing general structural formulas and features in the synthetic schemes are distinct from and should not be confused with the variables in the claims or the rest of the specification. These variables are meant only to illustrate how to make some of the compounds of this invention.

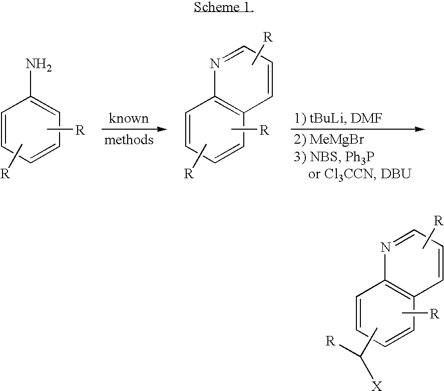

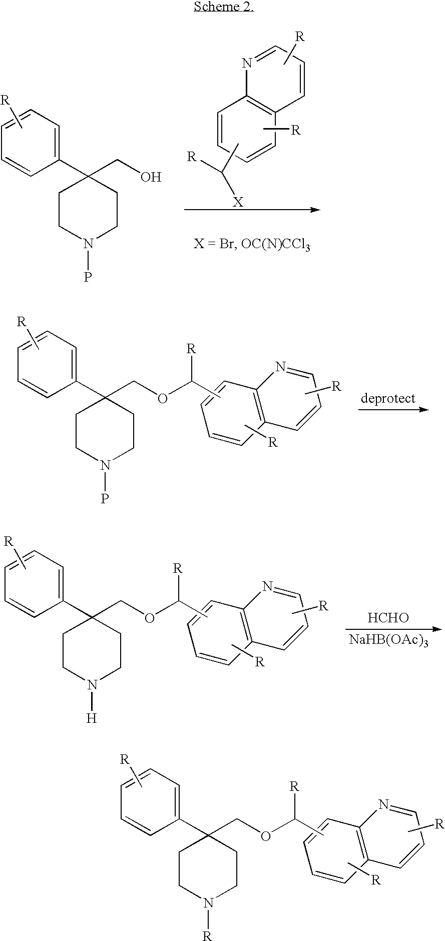

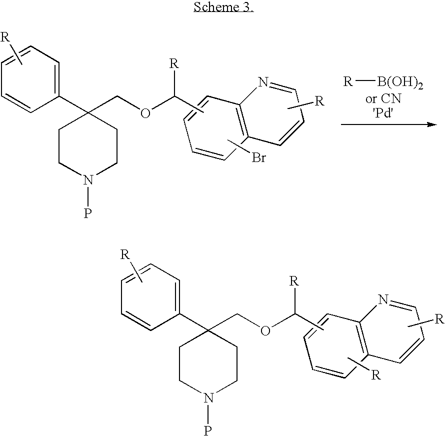

### Biological Methods

NK-1 Binding assay. Crude membrane suspensions were prepared for the NK1 and SERT radioligand binding assays from U373 cells or recombinant HEK-293 cells expressing hSERT, respectively. Cells were harvested from T-175 flasks as follows. The medium is removed from the flasks and the cells rinsed with HBSS without Ca and without Mg. The cells are then incubated for 5-10 minutes in 10 mM Tris-Cl, pH 7.5, 5 mM EDTA before the cells are lifted with a combination of pipetting and scraping, as needed. To prepare membranes, the cell suspension is collected into centrifuge bottles and homogenized for 30 seconds with a Polytron homogenizer. The suspension is centrifuged for 30 min @ 32,000×g, 4° C., then the supernatant is decanted and the pellet resuspended and homogenized in 50 mM Tris-Cl, pH 7.5, 1 mM EDTA for 10 seconds. The suspension is then centrifuged again for 30 min @ 32,000×g, 4° C. The supernatant is decanted and the pellet resuspended in 50 mM Tris-Cl, pH 7.5, 1 mM EDTA and briefly homogenized. A Bradford assay (Bio-rad) is performed and the membrane preparation diluted to 2 mg/ml with 50 mM Tris-Cl, pH 7.5, 1 mM EDTA. Aliquots are prepared, and then frozen and stored at −80° C.

NK1 radioligand binding assay. Compounds are dissolved in 100% DMSO at a concentration 100× the desired highest assay concentration, serially diluted 1:3 in 100% DMSO, and 0.6 ul/well of each solution is dispensed to a Nunc polypropylene, round bottom, 384 well plate. 100% inhibition is defined with 0.6 ul/well of 1 mM L-733,060 (Sigma L-137) dissolved in DMSO. 30 ul/well of a 2×U373 membrane preparation (267 ug/ml in 100 mM Tris-Cl, pH 7.5, 6 mM MgCl₂, 0.2% (v/v) Sigma mammalian protease inhibitor cocktail (Sigma P-8340), and 4 ug/ml chymostatin, Sigma C-7268) and 30 ul/well of a 2× radioligand solution (400 pM [¹²⁵I]Substance P (Perkin Elmer NEX-190) in 1% (w/v) BSA (Sigma A-2153), 0.1 mg/ml bacitracin, Sigma B-0125) are added to the well and the reaction incubated for 1 hour at room temperature. The contents of the assay plate are then transferred to a Millipore Multiscreen_{HTS} GF/B filter plate which has been pretreated with 0.5% PEI for at least one hour. The plate is vacuum filtered and washed with 7 washes of 100 ul/well of 20 mM Tris-Cl, pH 7.5, 0.5% (w/v) BSA chilled to 4° C. The filtration and washing is completed in less than 90 s. The plates are air-dried overnight, 12 ul/well of MicroScint scintillation fluid added, and the plates counted in a Trilux.

SERT radioligand binding assay. Compounds are dissolved in 100% DMSO at a concentration 100× the desired highest assay concentration, serially diluted 1:3 in 100% DMSO, and 0.4 ul/well of each solution is dispensed to a Nunc polypropylene, round bottom, 384 well plate. 100% inhibition is defined with 0.4 ul/well of 1 mM fluoxetine (Sigma F-132) dissolved in DMSO. 20 ul/well of a 2×HEK-hSERT membrane preparation (15 ug/ml in 50 mM Tris-Cl, pH 7.5, 120 mM NaCl, 5 mM KCl) and 20 ul/well of a 2× radioligand solution (520 pM [¹²⁵I]RTI-55 (Perkin-Elmer NEX-272) in 50 mM Tris-Cl, pH 7.5, 120 mM NaCl, 5 mM KCl) are added to each well and the reaction incubated for 1 hour at room temperature. The contents of the assay plate are then transferred to a Millipore Multiscreen_{HTS} GF/B filter plate which has been pretreated with 0.5% PEI for at least one hour. The plate is vacuum filtered and washed with 7 washes of 100 ul/well of 50 mM Tris-Cl, pH 7.5, 120 mM NaCl, 5 mM KCl chilled to 4° C. The filtration and washing is completed in less than 90 s. The plates are air-dried overnight, 12 ul/well of MicroScint scintillation fluid added, and the plates counted in a Trilux.

Data analysis. The raw data are normalized to percent inhibition using control wells defining 0% (DMSO only) and 100% (selective inhibitor) inhibition which are run on each plate. Each plate is run in triplicate, and the concentration response curve thus generated is fit using the four-parameter dose response equation, Y=Bottom+(Top-Bottom)/(1+10̂((LogIC₅₀−X)*HillSlope)) in order to determine the IC₅₀ value for each compound. The radioligand concentration chosen for each assay corresponds to the K_{d} concentration determined through saturation binding analysis for each assay. NK-1 and serotonin transporter binding results are shown in Table 1.

TABLE 1

| | | NK-1 IC₅₀ | SERT IC₅₀ |
|---|---|---|---|
| Example | Structure | (nM) | (nM) |
| 1 | 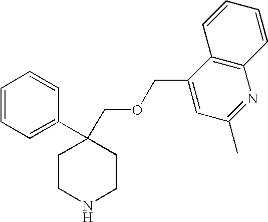 | C | A |
| 2 | 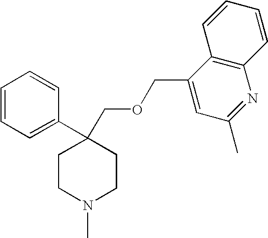 | C | A |
| 3 | 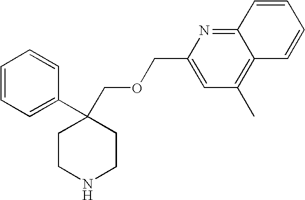 | B | A |
| 4 | 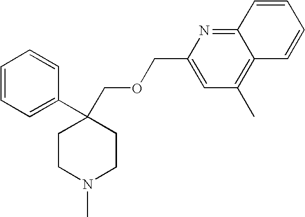 | B | A |
| 5 | 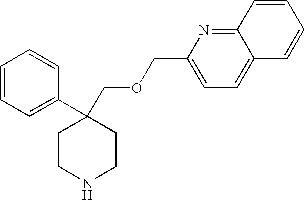 | C | A |
| 6 | 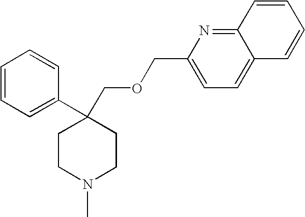 | C | A |
| 7 | 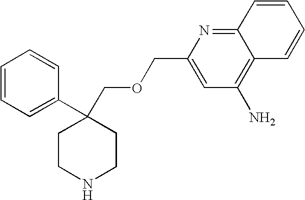 | C | A |
| 8 | 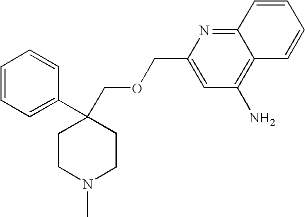 | C | A |
| 9 | 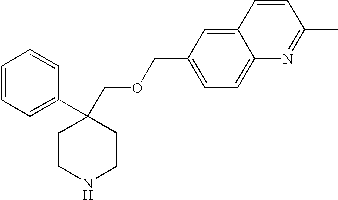 | C | A |
| 10 | 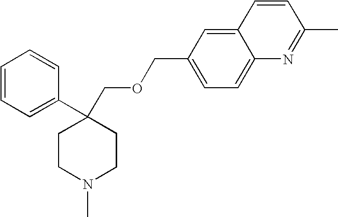 | C | A |
| 11 | 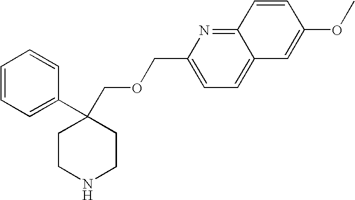 | C | A |
| 12 | 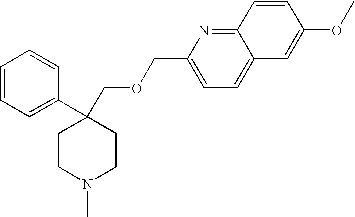 | C | A |
| 13 | 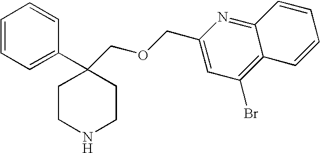 | B | A |
| 14 | 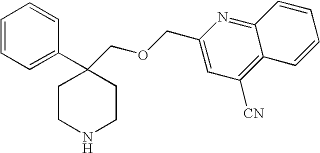 | C | A |
| 15 | 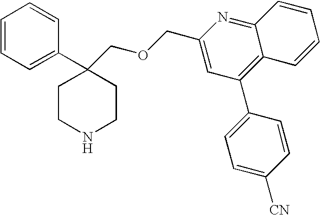 | C | A |
| 16 | 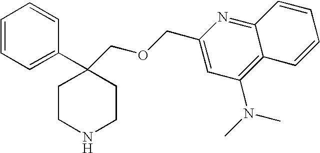 | C | A |
| 17 | 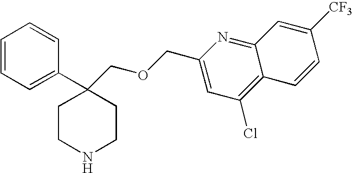 | C | A |
| 18 | 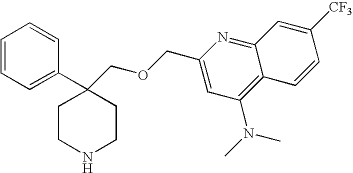 | C | A |
| 19 | 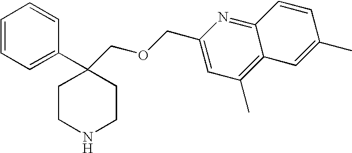 | C | A |
| 20 | 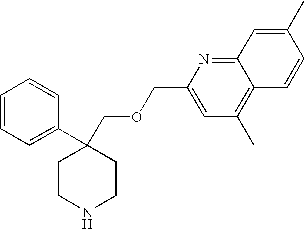 | C | A |
| 21 | 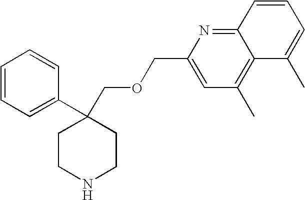 | C | A |
| 22 | 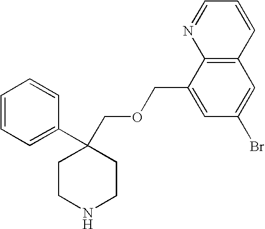 | A | A |
| 23 | 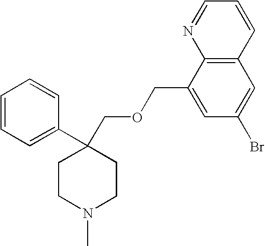 | A | A |
| 24 | 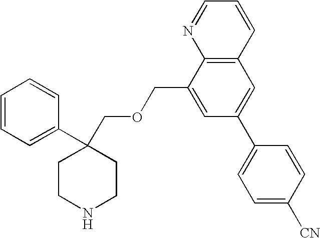 | C | A |
| 25 | 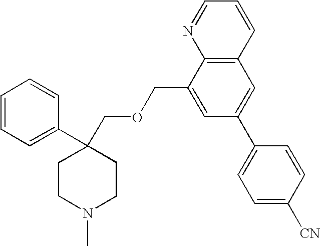 | C | A |
| 26 | 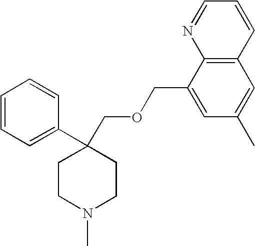 | A | A |
| 27 | 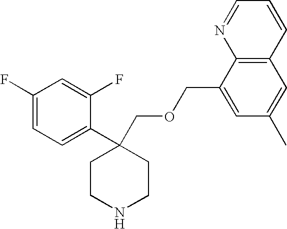 | A | A |
| 28 | 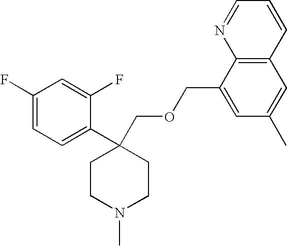 | B | A |
| 29 | 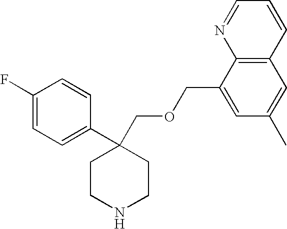 | A | A |
| 30 | 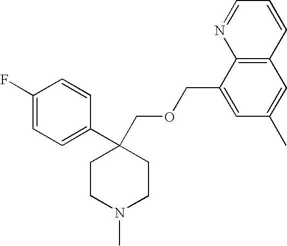 | A | A |
| 31 | 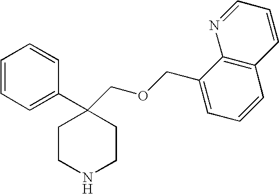 | C | A |
| 32 | 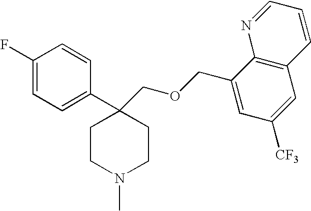 | A | A |
| 33 | 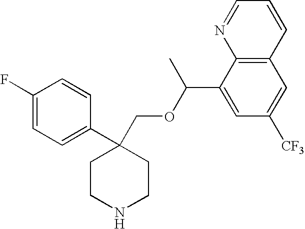 | A | A |
| 34 | 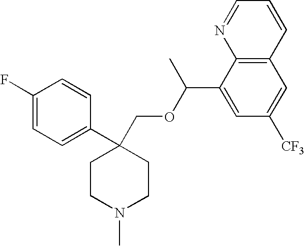 | A | A |
| 35 | 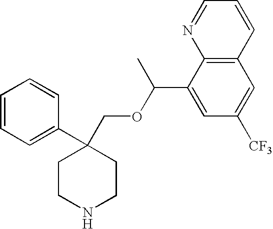 | A | A |
| 36 | 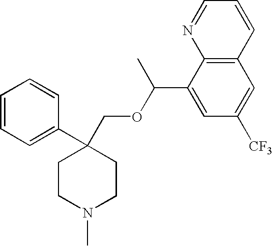 | A | A |
| 37 | 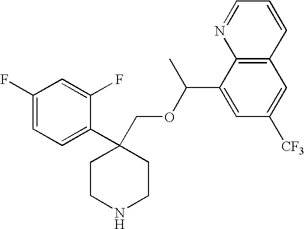 | A | A |
| 38 | 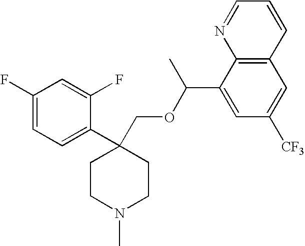 | A | A |
| 39 | 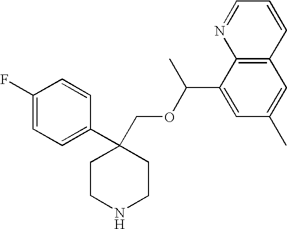 | A | A |
| 40 | 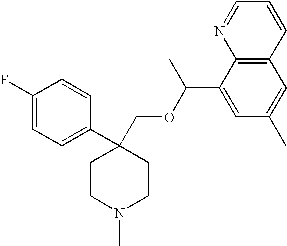 | A | A |
| 41 | 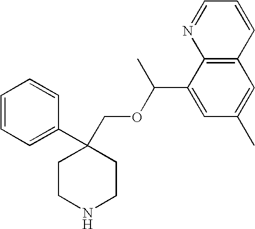 | A | A |
| 42 | 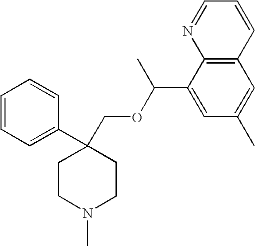 | A | A |
| 43 | 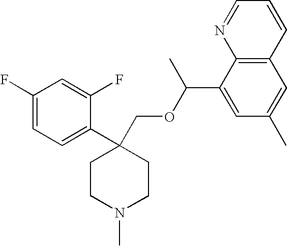 | A | A |
| 44 | 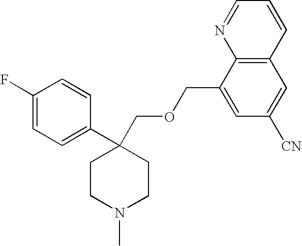 | C | A |
| 45 | 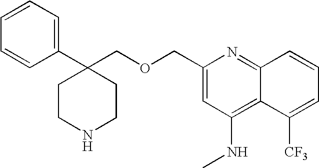 | C | A |
| 46 | 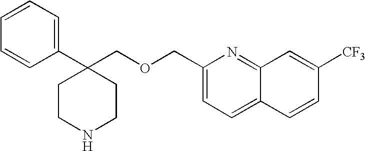 | C | A |
| 47 | 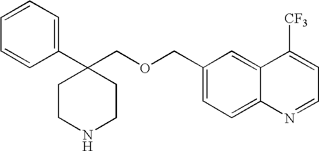 | C | A |
| 48 | 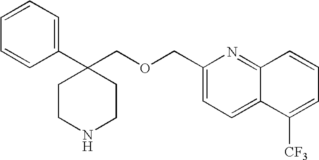 | C | A |
| 49 | 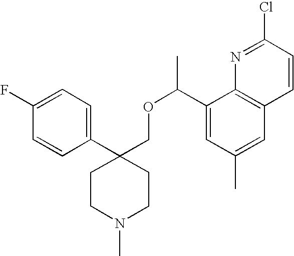 | A | A |
| 50 | 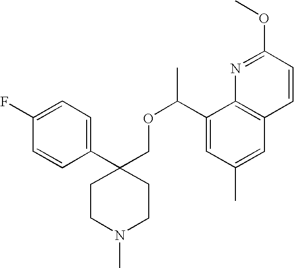 | A | A |
| 51 | 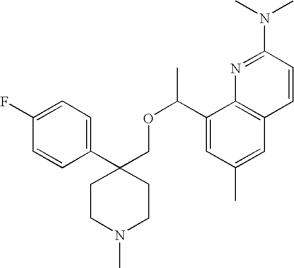 | A | B |
| 52 | 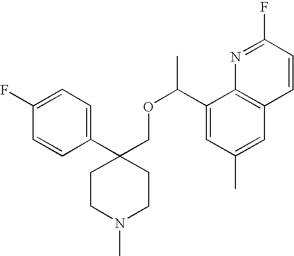 | A | A |
| 53 | 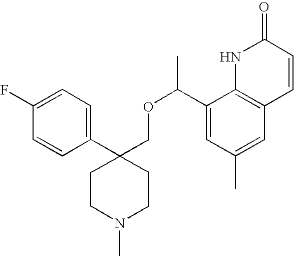 | A | A |

Values:
A = 0.01-100 nM;
B = 100-300 nM;
C >300 nM.

Some additional compounds are tabulated in Table 1a.

TABLE 1a

| | | | NK-1 | SERT |
|---|---|---|---|---|
| Example | Structure | enantiomer | IC₅₀ (nM) | IC₅₀ (nM) |
| 67 | 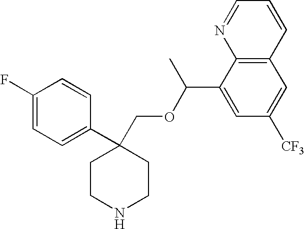 | 1 | C | A |
| 68 | 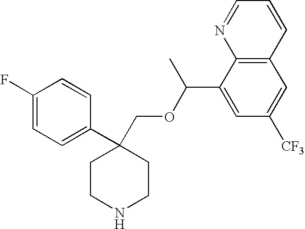 | 2 | A | A |
| 69 | 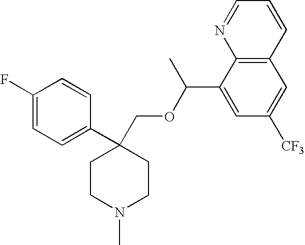 | 1 | C | A |
| 70 | 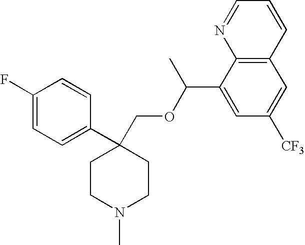 | 2 | A | A |
| 71 | 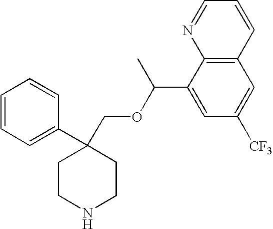 | 2 | A | A |
| 72 | 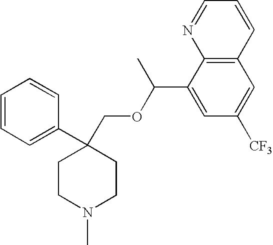 | 1 | C | A |
| 73 | 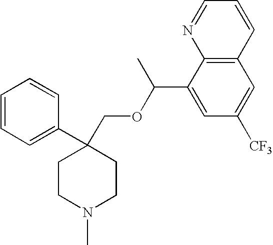 | 2 | A | A |
| 74 | 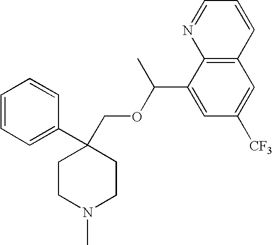 | 2 | A | A |
| 75 | 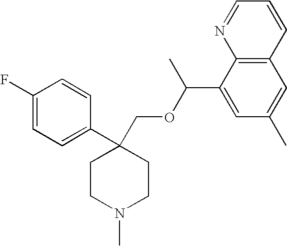 | 1 | C | A |
| 76 | 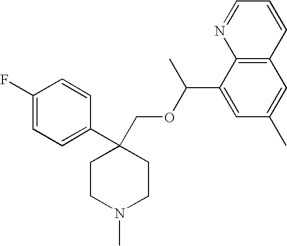 | 2 | A | A |
| 77 | 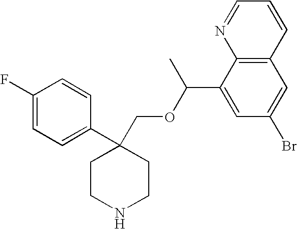 | 2 | A | A |
| 78 | 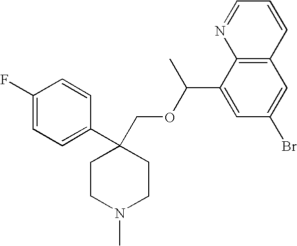 | 1 | B | A |
| 79 | 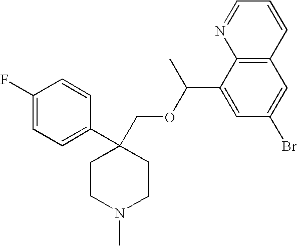 | 2 | A | A |

Values:
A = 0.01-100 nM;
B = 100-300 nM;
C >300 nM.

### Pharmaceutical Composition and Methods of Use

The compounds of Formula I demonstrate inhibition of neurokinin-1 or serotonin reuptake or both. Inhibition of these receptors correlates with efficacy for affective disorders such as anxiety, depression, obsessive compulsive disorder, bulimia, and panic disorder. As such, the compounds of Formula I can be useful for the treatment of these disorders and other aspects of the invention are compositions and methods of using the compounds to treat these conditions and other conditions associated with aberrant levels of tachykinins or serotonin or both.

The compounds of this invention are generally given as pharmaceutical compositions comprised of a therapeutically effective amount of a compound of Formula I, or a pharmaceutically acceptable salt, and a pharmaceutically acceptable carrier and may contain conventional exipients. A therapeutically effective amount is the amount needed to provide a meaningful patient benefit as determined by practitioners in that art. Pharmaceutically acceptable carriers are those conventionally known carriers having acceptable safety profiles. Compositions encompass all common solid and liquid forms including capsules, tablets, losenges, and powders as well as liquid suspensions, syrups, elixers, and solutions. Compositions are made using common formulation techniques and conventional excipients (such as binding and wetting agents) and vehicles (such as water and alcohols).

Solid compositions are normally formulated in dosage units providing from about 1 to about 1000 mg of the active ingredient per dose. Some examples of solid dosage units are 1 mg, 10 mg, 100 mg, 250 mg, 500 mg, and 1000 mg. Liquid compositions are generally in a unit dosage range of 1-100 mg/mL. Some examples of liquid dosage units are 1 mg/mL, 10 mg/mL, 25 mg/mL, 50 mg/mL, and 100 mg/mL. Generally, the dosage unit will be in a unit range similar to agents of that class used clinically, for example fluoxetine.

The invention encompasses all conventional modes of administration; oral and parenteral methods are preferred. Generally, the dosing regimen will be similar to agents of that class used clinically, for example fluoxetine. Typically, the daily dose will be 0.01-100 mg/kg body weight daily. Generally, more compound is required orally and less parenterally. The specific dosing regime, however, should be determined by a physician using sound medical judgement.

Tachykinin and serotonin modulators are associated with depression. Accordingly, another aspect of the invention are methods for treating depressive disorders including Major Depressive Disorders (MDD), bipolar depression, unipolar depression, single or recurrent major depressive episodes, recurrent brief depression, catatonic features, melancholic features including feeding disorders, such as anorexia, weight loss, atypical features, anxious depression, or postpartum onset. Other central nervous system disorders encompassed within the term MDD include neurotic depression, post-traumatic stress disorders (PTSD) and social phobia, with early or late onset dementia of the Alzheimer's type, with depressed mood, vascular dementia with depressed mood, mood disorders and tolerance induced by drugs such as alcohol, amphetamines, cocaine, inhalants, opioids, sedatives, anxiolytics and other substances, schizoaffective disorder of the depressed type, and adjustment disorder with depressed mood.

Tachykinin and serotonin modulators are also associated with the treatment or prevention of schizophrenic disorders. Accordingly, another aspect of the invention are methods for treating schizophrenic disorders including paranoid schizophrenia, disorganized schizophrenia, catatonic schizophrenia, undifferentiated schizophrenia, residual schizophrenia.

Tachykinin and serotonin modulators are also associated with the treatment or prevention of anxiety. Accordingly, another aspect of the invention are methods for treating anxiety disorders including panic disorders, agoraphobia, phobias, obsessive-compulsive disorder, stress disorders including post-traumatic stress disorders, generalized anxiety disorders, acute stress disorders and mixed anxiety-depression disorders.

Tachykinin and serotonin modulators are also associated with the treatment or prevention of cognitive disorders. Accordingly, another aspect of the invention are methods for treating cognitive disorders including dementia, and amnesia disorders. Tachykinin and serotonin modulators are also associated with the treatment or prevention of memory and cognition in healthy humans.

Tachykinin and serotonin modulators are also associated with use as analgesics. Accordingly, another aspect of the invention are methods for treating pain, including the treatment of traumatic pain such as postoperative pain, chronic pain such as arthritic pain such as occurring in osteo-, rheumatoid or psoriatic arthritis, neuropathic pain such as post-herpetic neuralgia, trigeminal neuralgia, segmental or intercostal neuralgia, fibromyalgia, peripheral neuropathy, diabetic neuropathy, chemotherapy-induced neuropathy, AIDS-related neuropathy, various forms of headache such as migraine, acute or chronic tension headache, cluster headaches, maxillary sinus pain, cancer pain, pain of bodily origin, gastrointestinal pain, sport's injury pain, dysmennorrhoea, menstrual pain, meningitis, musculoskeletal pain, low back pain e.g. spinal stenosis, prolapsed disc, sciatica, angina, ankylosing spondyolitis, gout, burns, scar pain, itch and thalamic pain such as post stroke thalamic pain.

Tachykinin and serotonin modulators are also associated with the treatment or prevention of sleep disorders. Accordingly, another aspect of the invention are methods for treating sleep disorders including insomnia, sleep apnea, narcolepsy, and circadian rhymic disorders.

Tachykinin and serotonin modulators are also associated with the treatment or prevention of inflammation. Accordingly, another aspect of the invention are methods for treating inflammation, including the treatment of inflammation in asthma, influenza and chronic bronchitis, in the treatment of inflammatory diseases of the gastrointestinal tract such as Crohn's disease, ulcerative colitis, inflammatory bowel disease and non-steroidal anti-inflammatory drug induced damage, inflammatory diseases of the skin such as herpes and eczema, inflammatory diseases of the bladder such as cystitis and urge incontinence, and eye and dental inflammation.

Tachykinin and serotonin modulators are also associated with the treatment or prevention of allergic disorders. Accordingly, another aspect of the invention are methods for treating allergic disorders, in particular allergic disorders of the skin such as urticaria, and allergic disorders of the airways such as rhinitis.

Tachykinin and serotonin modulators are also associated with the treatment or prevention of emesis, nausea, retching and vomiting. Accordingly, another aspect of the invention are methods for treating these disorders.

Tachykinin and serotonin modulators are also associated with the treatment or prevention of premenstrual dysphoric disorder (PMDD), in chronic fatigue syndrome and multiple sclerosis. Accordingly, another aspect of the invention are methods for treating these disorders.

### DESCRIPTION OF SPECIFIC EMBODIMENTS

The following experimental procedures describe the synthesis of some Formula I compounds. Standard chemistry conventions are used in the text unless otherwise noted. The experimental encompass reasonable variations known in the art.

HPLC method 1: Phenomenex Luna 3.0×50 mm, A=90% H₂O/10% MeOH, B=90% MeOH/10% H₂O, Modifier 0.1% TFA, 0.00 min=0% B, 2.0 min=100% B, Flow rate=4 mL/min. HPLC method 2: Phenomenex Luna 3.0×50 mm, A=90% H₂O/10% MeOH, B=90% MeOH/10% H₂O, Modifier 0.1% TFA, 0.00 min=0% B, 3.0 min=100% B, Flow rate=4 mL/min. HPLC method 3: Phenomenex Luna 3.0×50 mm, A=90% H₂O/10% MeOH, B=90% MeOH/10% H₂O, Modifier 0.1% TFA, 0.00 min=0% B, 4.0 min=100% B, Flow rate=4 mL/min.

### Intermediate 1

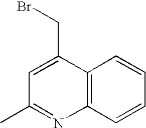

4-(bromomethyl)-2-methylquinoline. To a solution of (2-methyl-quinolin-4-yl)-methanol (200 mg, 1.15 mmol) in THF (8 ml) at 0° C. was added NBS (431 mg, 2.42 mmol) and triphenylphosphine (605 mg, 2.31 mmol). The reaction mixture was allowed to warm to room temperature and stirred reaction overnight. The reaction mixture was quenched with sat NaHCO₃ and extracted with ethyl acetate. The organic portions were dried over MgSO₄, filtered and concentrated. Column chromatography on silica gel (10-30% ethyl acetate/hexanes) afforded intermediate 1 (258 mg, 1.09 mmol, 94%). ¹H NMR (400 MHz, CDCl₃) δ ppm 7.95-8.18 (m, 2H), 7.65-7.81 (m, 1H), 7.43-7.66 (m, 1H), 7.32 (s, 1H), 4.82 (s, 2H), 2.73 (s, 3H).

### Example 1

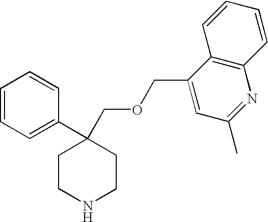

2-methyl-4-(((4-phenylpiperidine-4-yl)methoxy)methyl)quinoline. To a cold solution of tert-butyl 4-(hydroxymethyl)-4-phenylpiperidine-1-carboxylate (100 mg, 0.343 mmol) and 4-(bromomethyl)-2-methylquinoline (121 mg, 0.514 mmol) in THF (3.5 ml) was added 1.0 equivalent of potassium tert-butoxide (38 mg, 0.338 mmol). The reaction mixture was stirred at 0° C. for 20 minutes. A further 1.0 equivalent of potassium tertbutoxide (38 mg, 0.338 mmol) was added. The mixture was allowed to slowly warm to room temperature over 2 hours. The reaction mixture was quenched with sat NH₄Cl and extracted with ethyl acetate. The combined organics were washed with brine, dried over MgSO₄ and concentrated. Column chromatography on silica gel (10-30% ethyl acetate/hexanes) afforded Boc-protected intermediate of Example 1 (126 mg, 0.282 mmol, 82%). This intermediate was then treated with a trifluoroacetic acid/methylene chloride mixture (1:2, 2 ml) for 40 minutes. The solvent was removed in vacuo and the resulting crude mixture passed through a cation exchange column (Waters MCX, 1 g). After washing the column with several volumes of methanol, the product was eluted by washing the column with 2 M ammonia in methanol to yield the title compound (81 mg, 0.233 mmol, 83%). ¹H NMR (400 MHz, CDCl₃) δ ppm 7.98 (br. d, J=8.06 Hz, 1H), 7.68 (br. d, J=8.31 Hz, 1H), 7.59-7.66 (m, 1H), 7.32-7.41 (m, 6H), 7.03 (s, 1H), 4.72-4.81 (m, 2H), 3.43-3.52 (m, 2H), 2.89-3.00 (m, 2H), 2.70-2.84 (m, 2H), 2.63-2.69 (m, 3H), 2.13-2.28 (m, 2H), 1.91-2.03 (m, 2H). Mass 347 [M+H]⁺.

### Example 2

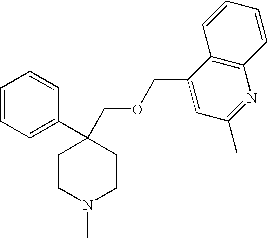

2-methyl-4-(((1-methyl-4-phenylpiperidin-4-yl)methoxy)methyl)quinoline. To a solution of 2-methyl-4-(((4-phenylpiperidine-4-yl)methoxy)methyl)quinoline (45 mg, 0.129 mmol) in DCM (0.5 ml) was added formaldehyde (37 wt % in water) (0.5 ml) and sodium triacetoxyborohydride (110 mg, 0.519 mmol). The mixture was stirred at room temperature overnight then quenched with sat NaHCO₃ and extracted into EtOAc. The combined organics were washed with brine, dried over MgSO₄ and concentrated. The crude material was purified by column chromatography on silica gel (0-12%) MeOH in DCM to afford the title compound in example 2 (18 mg, 0.049 mmol, 39%). ¹H NMR (400 MHz, MeOD) δ ppm 7.85 (d, J=8.31 Hz, 1H), 7.74 (d, J=8.31 Hz, 1H), 7.57-7.65 (m, 1H), 7.33-7.42 (m, 3H), 7.24-7.31 (m, 2H), 7.17 (t, J=7.30 Hz, 1H), 7.09 (s, 1H), 3.52 (s, 2H), 3.26-3.34 (m, 2H), 2.55 (s, 5H), 2.14 (s, 7H), 2.00 (s, 2H). Mass 361 [M+H]⁺.

### Intermediate 2

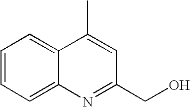

(4-methylquinolin-2-yl)methanol. To a solution of 4-methylquinoline (2.00 g, 14.0 mmol) in 50 ml of water was added ethylene glycol (50 ml), trifluoroacetic acid (1.08 ml, 14.0 mmol), ammonium persulfate (12.75 g, 55.9 mmol) and silver nitrate (237 mg, 1.40 mmol). The mixture was heated to reflux for 3 hours. The solution was cooled and made basic by addition of NaOH (10 N) then extracted with dichloromethane, dried over Na₂SO₄ and concentrated. Column chromatography on silica gel (30%) EtOAc in hexanes afforded intermediate 2 (760 mg, 4.38 mmol, 31%).

Table 2 describes compounds that were prepared from (4-methylquinolin-2-yl)methanol following the experimental conditions described in intermediate 1, example 1 and example 2. Retention time (t_{R}) is in min.

TABLE 2

| | | MS | HPLC | |
|---|---|---|---|---|
| Example | Structure | [M + H]⁺ | (method) | ¹H NMR |
| 3 | 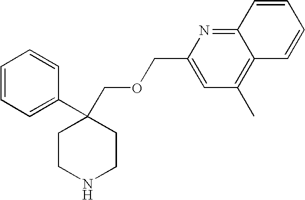 | 347.28 | 1.557(2) | ¹H NMR (400 MHz,CDCl₃) δ ppm 7.91-8.01 (m, 2 H), 7.63-7.69 (m, 1 H), 7.48-7.54 (m, 1 H), 7.33-7.41 (m, 4 H), 7.26-7.30 (m, 1 H), 6.95-6.99 (m, 1 H), 4.59-4.66 (m, 2 H), 3.49 (d,J = 7.55 Hz, 2 H), 2.93-3.02 (m, 2 H), 2.70-2.85 (m, 2 H), 2.57-2.61 (m, 3 H), 2.21 (s, 2H), 1.92-2.08 (m, 2 H) |
| 4 | 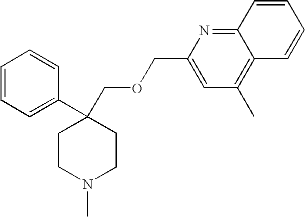 | 361.32 | 1.035(1) | ¹H NMR (400 MHz,MeOD) δ ppm 7.99 (d,J = 8.31 Hz, 1 H), 7.85 (d,J = 8.56 Hz, 1 H), 7.61-7.68 (m, 1 H), 7.48-7.55 (m, 1 H), 7.34-7.39 (m, 2 H), 7.25-7.32 (m, 2 H), 7.15-7.21 (m, 1 H), 6.97 (s, 1H), 4.50 (s, 2 H), 3.47 (s,2 H), 2.63 (s, 3 H), 2.60-2.71 (m, 2 H), 2.16-2.30 (m, 4 H), 2.12 (s, 3H), 2.00 (s, 2 H) |

### Intermediate 3

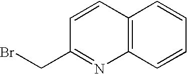

2-(bromomethyl)quinoline. To a solution of quinaldine (500 mg, 3.49 mmol) in CCl₄ (50 ml) was added NBS (621 mg, 3.49 mmol) and AIBN (28 mg, 0.174 mmol). This mixture was refluxed overnight. The reaction was cooled to 0° C. and filtered to remove any undissolved solids. The filtrate was concentrated under vacuum. Column chromatography on silica gel (0-12% ethyl acetate/hexanes) afforded intermediate 3 (71 mg, 0.319 mmol, 10%). ¹H NMR (400 MHz, CDCl₃) δ ppm 8.18 (d, J=8.31 Hz, 1H), 8.07 (d, J=8.56 Hz, 1H), 7.81 (d, J=8.06 Hz, 1H), 7.69-7.77 (m, 1H), 7.51-7.60 (m, 2H), 4.71 (s, 2H). Mass 222 [M+H]⁺.

Table 3 describes compounds that were prepared from 2-(bromomethyl)quinoline following the experimental conditions described in example 1 and example 2. Retention time (t_{R}) is in min.

TABLE 3

| | | MS | HPLC | |
|---|---|---|---|---|
| Example | Structure | [M + H]⁺ | (method) | ¹H NMR |
| 5 | 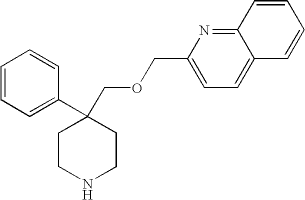 | 333.28 | 1.017(1) | ¹H NMR (400 MHz,MeOD) δ ppm 8.14(d, J = 8.56 Hz, 1 H),7.84 (dd, J = 19.77,8.44 Hz, 2 H), 7.62-7.69 (m, 1 H), 7.49 (t,J = 7.55 Hz, 1 H), 7.33-7.39 (m, 2 H), 7.20-7.31 (m, 3 H), 1.81-1.96 (m, 2 H) 7.15 (t,J = 7.30 Hz, 1 H), 4.54(s, 2 H), 3.47 (s, 2 H),2.75-2.85 (m, 2 H),2.53-2.68 (m, 2 H),2.16 (s, 2 H) |
| 6 | 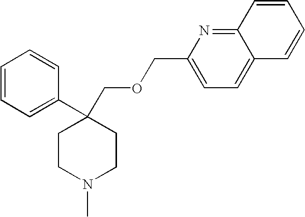 | 347.30 | 0.993(1) | ¹H NMR (400 MHz,MeOD) δ ppm 8.14(d, J = 8.56 Hz, 1 H),7.84 (dd, J = 19.26,8.18 Hz, 2 H), 7.62-7.69 (m, 1 H), 7.49 (t,J = 7.55 Hz, 1 H), 7.33-7.39 (m, 2 H), 7.20-7.30 (m, 3 H), 7.15 (t,J = 7.18 Hz, 1 H), 4.54(s, 2 H), 3.48 (s, 2 H),2.53-2.61 (m, 2 H),2.17-2.35 (m, 2 H),2.17 (s, 3 H), 2.00-2.11 (m, 2 H) |

### Intermediate 4

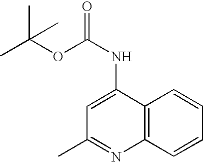

tert-butyl 2-methylquinolin-4-ylcarbamate. To a solution of 4-aminoquinaldine (1.00 g, 6.32 mmol) in THF (30 ml) was added (BOC)₂O (1.37 g, 6.32 mmol), TEA (1.06 ml, 7.58 mmol), and DMAP (77 mg, 0.632 mmol). The reaction mixture was stirred overnight then quenched with water and extracted into EtOAc. The combined organics were washed with brine, dried over MgSO₄ and concentrated under vacuum. Column chromatography on silica gel (20-40% ethyl acetate/hexanes) afforded intermediate 4 (517 mg, 2.00 mmol, 31%). ¹H NMR (400 MHz, CDCl₃) δ ppm 7.99-8.07 (m, 2H), 7.64-7.76 (m, 2H), 7.48 (t, J=7.68 Hz, 1H), 2.71 (s, 3H), 1.58 (s, 9H).

### Intermediate 5

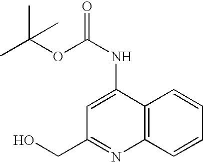

tert-butyl 2-(hydroxymethyl)quinolin-4-ylcarbamate. To a solution of tert-butyl 2-methylquinolin-4-ylcarbamate (410 mg, 1.58 mmol) in dry 1,4-dioxane (15 ml) was added selenium (IV) dioxide (208 mg, 1.87 mmol). The mixture was heated to reflux for 3 hours then cooled to room temperature and filtered through a pad of celite. The filter cake was rinsed with EtOH. The filtrate was cooled to 0° C. and NaBH₄ (60 mg, 1.58 mmol) was added. The reaction mixture was stirred at room temperature for 3 hours then slowly quenched with sat NH₄Cl. This mixture was concentrated under vacuum to remove EtOH and dioxane. The resulting mixture was suspended in water and extracted with EtOAc. The combine organic portions were washed with brine, dried over MgSO₄ and concentrated under vacuum. Column chromatography on silica gel (30-50% ethyl acetate/hexanes) afforded intermediate 5 (345 mg, 1.25 mmol, 79%). ¹H NMR (400 MHz, CDCl₃) δ ppm 8.01-8.09 (m, 2H), 7.68-7.79 (m, 2H), 7.51-7.58 (m, 1H), 7.31 (s, 1H), 4.87 (s, 2H), 1.53-1.62 (m, 9H). Mass: 275 [M+H]⁺.

Table 4 describes compounds that were prepared from tert-butyl 2-(hydroxymethyl)quinolin-4-ylcarbamate following the experimental conditions described in intermediate 1, example 1 and example 2. Retention time (t_{R}) is in min.

TABLE 4

| | | MS | HPLC | |
|---|---|---|---|---|
| Example | Structure | (MH)⁺ | (method) | ¹H NMR |
| 7 | 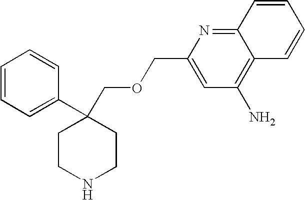 | 348.20 | 0.948(1) | ¹H NMR (400 MHz,MeOD) δ ppm) 8.19 (d,J = 8.56 Hz, 1 H), 7.83 (t,J = 7.68 Hz, 1 H, 7.68 (d,J = 8.56 Hz, 1 H), 7.57 (t,J = 7.81 Hz, 1 H, 7.35-7.42 (m, 2 H), 7.30 (t,J = 7.81 Hz, 2 H), 7.17 (t,J = 7.30 Hz, 1 H), 6.63 (s,1 H), 4.48 (s, 2 H), 3.54 (s, 2H), 3.25-3.31 (m, 2 H),2.78-2.96 (m, 2 H), 2.47(d, J = 14.86 Hz, 2 H), 2.01-2.25 (m, 2 H) |
| 8 | 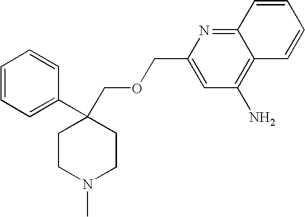 | 362.29 | 1.035(1) | ¹H NMR (400 MHz,MeOD) δ ppm 7.95 (d,J = 8.31 Hz, 1 H), 7.66 (d,J = 8.56 Hz, 1 H), 7.50-7.58 (m, 1 H), 7.23-7.39(m, 5 H), 7.14 (t, J = 7.30Hz, 1 H), 6.48 (s, 1 H),4.35 (s, 2 H), 3.44 (s, 2 H),2.64-2.73 (m, 2 H), 2.23-2.34 (m, 4 H), 2.21 (s, 3H), 2.05-2.16 (m, 2 H) |

Table 5 describes compounds that were prepared from 6-(bromomethyl)-2-methylquinoline following the experimental conditions described in example 1 and example 2. Retention time (t_{R}) is in min.

TABLE 5

| | | MS | HPLC | |
|---|---|---|---|---|
| Example | Structure | (MH)⁺ | (method) | ¹H NMR |
| 9 | 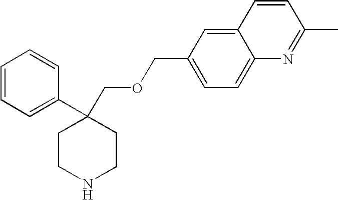 | 347.23 | 1.025(1) | ¹H NMR (400 MHz,MeOD) δ ppm 8.06(d, J = 8.56 Hz, 1 H),7.78 (d, J = 8.56 Hz, 1H), 7.54 (s, 1 H),7.46 (dd, J = 8.69,1.89 Hz, 1 H), 7.30-7.41 (m, 5 H), 7.21-7.28 (m, 1 H), 4.51(s, 2 H), 3.39-3.46(m, 2 H), 3.18 (d,J = 13.60 Hz, 2 H),2.76-2.93 (m, 2 H),2.64 (s, 3 H), 2.41-2.49 (m, 2 H), 2.01-2.20 (m, 2 H) |
| 10 | 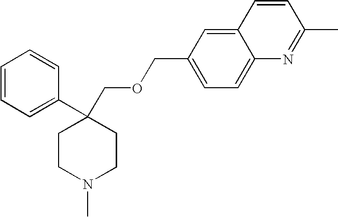 | 361.26 | 1.493(2) | ¹H NMR (400 MHz,MeOD) δ ppm 8.03(d, J = 8.31 Hz, 1 H),7.75 (d, J = 8.81 Hz, 1H), 7.47 (s, 1 H),7.24-7.42 (m, 6 H),7.17 (t, J = 7.18 Hz, 1H), 4.46 (s, 2 H),3.41 (s, 2 H), 1.92-2.52-2.66 (m, 5 H),2.16-2.32 (m, 4 H),2.12 (s, 3 H), 2.06(m, 2 H) |

Table 6 describes compounds that were prepared from 6-methoxyquinaldine following the experimental conditions described in example 1 and example 2. Retention time (t_{R}) is in min.

TABLE 6

| | | MS | HPLC | |
|---|---|---|---|---|
| Example | Structure | (MH)⁺ | (method) | ¹H NMR |
| 11 | 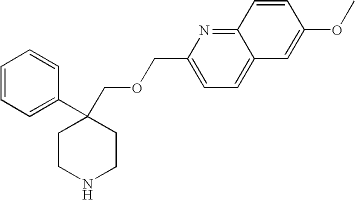 | 363.25 | 1.073(1) | ¹H NMR (400 MHz, MeOD) δppm 8.06 (d, J = 8.31 Hz, 1 H),7.76 (d, J = 9.06 Hz, 1 H), 7.34-7.39 (m, 2 H), 7.24-7.33 (m, 3H), 7.13-7.21 (m, 3 H), 4.50 (s,2 H), 3.86 (s, 3 H), 3.46 (s, 2 H),2.76-2.90 (m, 2 H), 2.53-2.70(m, 2 H), 2.20-2.28 (m, 2 H),1.84-2.01 (m, 2 H) |
| 12 | 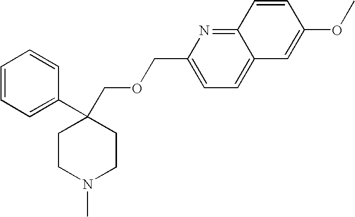 | 377.27 | 1.052(1) | ¹H NMR (400 MHz, MeOD) δppm 8.05 (d, J = 8.56 Hz, 1 H),7.76 (d, J = 9.07 Hz, 1 H), 7.33-7.39 (m, 2 H), 7.24-7.32 (m, 3H), 7.12-7.20 (m, 3 H), 450 (s,2 H), 385 (s,3 H), 3.41-3.49(m,2 H), 2.66-2.75 (m, 2 H),2.19-2.33 (m, 4 H), 2.18 (s, 3H), 2.04 (d,UJ = 10.83 Hz, 2 H) |

### Intermediate 6

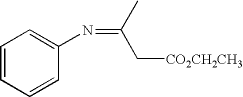

(Z)-ethyl 3-(phenylimino)butanoate. A mixture of aniline (5.00 g, 53.6 mmol), ethyl acetoacetate (6.84 ml, 53.6 mmol), absolute EtOH (20 ml), glacial AcOH (0.06 ml) and drierite (7.00 g) were heated under reflux for 4 hours. The drierite was filtered off and the solvent was removed under vacuum. Column chromatography on silica gel (0-10% ethyl acetate/hexanes) afforded intermediate 6 (6.26 g, 30.5 mmol, 56%). ¹H NMR (400 MHz, CDCl₃) δ ppm 7.31 (t, J=7.81 Hz, 2H), 7.11-7.17 (m, 1H), 7.07 (d, J=7.55 Hz, 2H), 4.68 (s, 1H), 4.05-4.21 (m, 2H), 1.99 (s, 3H), 1.28 (t, J=7.05 Hz, 3H).

### Intermediate 7

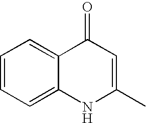

2-methylquinolin-4(1H)-one. (Z)-ethyl 3-(phenylimino)butanoate (6.26 g, 30.49 mmol) was cyclized in diphenyl ether (30 ml) by heating at 255° C. for 30 minutes. After cooling to room temperature a precipitate formed. The precipitate was filtered and washed several times with ether to remove any traces of phenyl ether to afford intermediate 7 (2.6 g, 16.3 mmol, 53%). ¹H NMR (400 MHz, MeOD) δ ppm 8.12 (d, J=8.06 Hz, 1H), 7.55-7.66 (m, 1H), 7.46 (d, J=8.31 Hz, 1H), 7.31 (t, J=7.68 Hz, 1H), 6.13 (s, 1H), 2.39 (s, 3H). Mass 159 [M+H]⁺.

### Intermediate 8

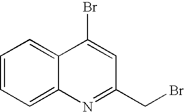

4-bromo-2-(bromomethyl)quinoline. 2-methylquinolin-4(1H)-one (1.00 g, 6.28 mmol) was slowly added to phosphorous oxybromide (1.80 g, 6.28 mmol) while the temperature was raised from 75° C. to 150° C. The mixture was stirred at 150° C. for 2.5 hours then quenched with ice water. The product was extracted into EtOAc, dried over MgSO₄ and concentrated. Column chromatography on silica gel (0-10% ethyl acetate/hexanes) afforded intermediate 8 (363 mg, 1.20 mmol, 19%). ¹H NMR (400 MHz, CDCl₃) δ ppm 8.17 (d, J=8.06 Hz, 1H), 8.05 (d, J=8.31 Hz, 1H), 7.87 (s, 1H), 7.74-7.81 (m, 1H), 7.60-7.68 (m, 1H), 4.65 (s, 2H). Mass 299 [M+H]⁺.

### Example 13

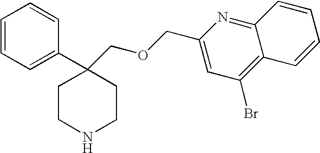

4-bromo-2-(((4-phenylpiperidin-4-yl)methoxy)methyl)quinoline Example 13 was prepared from 4-bromo-2-(bromomethyl)quinoline following the experimental conditions described in example 1. ¹H NMR (400 MHz, MeOD) δ ppm 8.12 (d, J=8.56 Hz, 1H), 7.90 (d, J=8.31 Hz, 1H), 7.70-7.77 (m, 1H), 7.56-7.66 (m, 1H), 7.47 (s, 1H), 7.35-7.41 (m, 2H), 7.31 (t, J=7.81 Hz, 2H), 7.19 (t, J=7.18 Hz, 1H), 4.53 (s, 2H), 3.51 (s, 2H), 2.81-2.95 (m, 2H), 2.57-2.74 (m, 2H), 2.23 (s, 2H), 1.86-2.00 (m, 2H). Mass 411 [M+H]⁺.

### Example 14

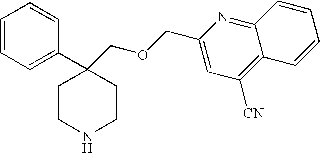

2-(((4-phenylpiperidin-4-yl)methoxy)methyl)quinoline-4-carbonitrile. A solution of tert-butyl 4-(((4-bromoquinolin-2-yl)methoxy)methyl)-4-phenylpiperidine-1-carboxylate (77 mg, 0.150 mmol) in DMF (2 ml) and water (40 uL) was degassed by bubbling through N₂ for 5 minutes. To this solution was added Pd₂ dba₃ (14 mg, 0.015 mmol) and dppf (17 mg, 0.030 mmol), followed by zinc cyanide (17 mg, 0.150 mmol). The mixture was heated at 120° C. overnight. The reaction mixture was filtered through a pad of celite and washed with EtOAc. The filtrate was washed with water and brine then dried over MgSO₄ and concentrated. Column chromatography on silica gel (10-30% ethyl acetate/hexanes) afforded Boc-protected intermediate of example 14 (15 mg, 0.032 mmol, 22%). This intermediate was then treated with a trifluoroacetic acid/methylene chloride mixture (1:2, 2 ml) for 40 minutes. The solvent was removed in vacuo and the resulting crude mixture passed through a strong cation exchange column (Waters MCX, 1 g). After washing the column with several volumes of methanol, the product was eluted with 2 M ammonia in methanol to afford the title compound in example 14 (9 mg, 0.025 mmol, 81%). ¹H NMR (400 MHz, MeOD) δ ppm 8.06 (d, J=8.31 Hz, 1H), 8.00 (d, J=8.31 Hz, 1H), 7.79-7.85 (m, 1H), 7.68-7.76 (m, 1H), 7.29-7.41 (m, 5H), 7.19-7.26 (m, 1H), 4.59 (s, 2H), 3.46-3.58 (m, 2H), 2.89-3.02 (m, 2H), 2.60-2.81 (m, 2H), 2.31-2.39 (m, 2H), 1.87-2.07 (m, 2H). Mass 358 [M+H]⁺.

### Example 15

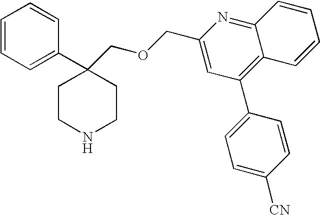

4-(2-(((4-phenylpiperidin-4-yl)methoxy)methyl)quinolin-4-yl)benzonitrile. A mixture of tert-butyl 4-(((4-bromoquinolin-2-yl)methoxy)methyl)-4-phenylpiperidine-1-carboxylate (50 mg, 0.097 mmol), 4-cyanophenyl boronic acid (60 mg, 0.410 mmol), and Pd(PPh₃)₄ (11 mg, 0.0097 mmol) were combined in THF (2 ml) in a microwave sealed vial. The mixture was flushed with N₂, then 0.25 ml of 1N KOH aq solution was added. The mixture was heated in a microwave reactor at 120° C. for 1 hour. After cooling, the crude mixture was diluted with EtOAc and washed with water and brine then dried over MgSO₄ and concentrated. Column chromatography on silica gel (10-40% ethyl acetate/hexanes) afforded Boc-protected intermediate of example 15 (38 mg, 0.071 mmol, 73%). This intermediate was then treated with a trifluoroacetic acid/methylene chloride mixture (1:2, 2 ml) for 40 minutes. The solvent was removed in vacuo and the resulting crude mixture passed through a strong cation exchange column (Waters MCX, 1 g). After washing the column with several volumes of methanol, the product was eluted with 2 M ammonia in methanol to afford the title compound in example 15 (30 mg, 0.069 mmol, 96%). ¹H NMR (400 MHz, MeOD) δ ppm 7.98 (d, J=8.06 Hz, 1H), 7.90 (d, J=8.31 Hz, 2H), 7.72 (t, J=7.93 Hz, 2H), 7.46-7.59 (m, 3H), 7.35 (d, J=8.31 Hz, 2H), 7.20 (t, J=7.81 Hz, 2H), 6.98-7.08 (m, 2H), 4.64 (s, 2H), 3.53 (s, 2H), 2.99-3.16 (m, 2H), 2.67-2.91 (m, 2H), 2.35 (s, 2H), 1.86-2.13 (m, 2H). Mass 434 [M+H]⁺.

### Example 16

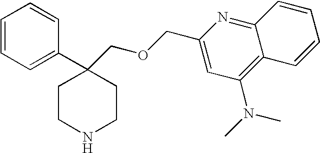

N,N-dimethyl-2-(((4-phenylpiperidin-4-yl)methoxy)methyl)quinolin-4-amine. To a mixture of tert-butyl 4-(((4-bromoquinolin-2-yl)methoxy)methyl)-4-phenylpiperidine-1-carboxylate (56 mg, 0.109 mmol) in EtOH (2 ml) was added dimethylamine (2.0 M soln in THF) (0.11 ml, 0.218 mmol). This mixture was heated to reflux over 68 hours then concentrated under vacuum and purified by column chromatography on silica gel (40-60% ethyl acetate/hexanes) to afford Boc-protected intermediate of example 16 (32 mg, 0.067 mmol, 61%). This intermediate was then treated with a trifluoroacetic acid/methylene chloride mixture (1:2, 2 ml) for 40 minutes. The solvent was removed in vacuo and the resulting crude mixture was passed through a strong cation exchange column (Waters MCX, 1 g). After washing the column with several volumes of methanol, the product was eluted with 2 M ammonia in methanol to afford the title compound in example 16 (23 mg, 0.061 mmol, 92%). ¹H NMR (400 MHz, MeOD) δ ppm 8.00 (d, J=8.56 Hz, 1H), 7.77 (d, J=8.31 Hz, 1H), 7.53-7.60 (m, 1H), 7.36-7.45 (m, 3H), 7.31 (t, J=7.68 Hz, 2H), 7.19 (d, J=7.05 Hz, 1H), 6.65 (s, 1H), 4.47 (s, 2H), 3.49 (s, 2H), 3.00 (s, 2H), 2.92 (s, 6H), 2.63-2.79 (m, 2H), 2.28 (s, 2H), 1.99-2.08 (m, 2H). Mass 376 [M+H]⁺.

### Intermediate 9

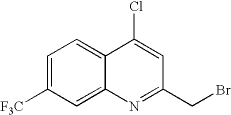

2-(bromomethyl)-4-chloro-7-(trifluoromethyl)quinoline. To a solution of 4-chloro-2-methyl-7-(trifluoromethyl) quinoline (250 mg, 1.01 mmol) in CCl₄ (10 ml) was added NBS (199 mg, 1.11 mmol) and AIBN (13 mg, 0.081 mmol). This mixture was heated to reflux overnight, cooled and diluted with DCM then quenched with 10% aq NaHSO₃. The layers were separated and the DCM layer was washed with sat NaHCO₃ and brine then dried over MgSO₄ and concentrated. Column chromatography on silica gel (0-8% ethyl acetate/hexanes) afforded intermediate 9 (163 mg, 0.502 mmol, 49%). ¹H NMR (400 MHz, CDCl₃) δ ppm 8.26-8.49 (m, 2H), 7.64-7.92 (m, 2H), 4.66 (s, 2H). Mass 324 [M+H]⁺.

### Example 17

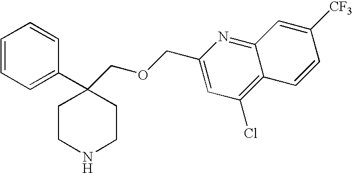

4-chloro-2-(((4-phenylpiperidin-4-yl)methoxy)methyl)-7-(trifluoromethyl)quinoline. Example 17 was prepared from 2-(bromomethyl)-4-chloro-7-(trifluoromethyl)quinoline following the experimental conditions described in example 1. ¹H NMR (400 MHz, MeOD) δ ppm 8.35 (d, J=8.81 Hz, 1H), 8.21 (s, 1H), 7.83 (dd, J=8.81, 1.76 Hz, 1H), 7.28-7.42 (m, 5H), 7.19 (t, J=7.30 Hz, 1H), 4.57 (s, 2H), 3.52 (s, 2H), 2.88-3.00 (m, 2H), 2.61-2.79 (m, 2H), 2.27 (s, 2H), 1.88-2.04 (m, 2H). Mass 435 [M+H]⁺.

### Example 18

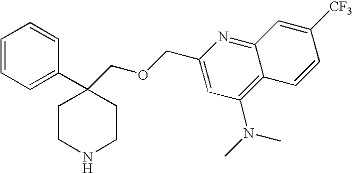

N,N-dimethyl-2-(((4-phenylpiperidin-4-yl)methoxy)methyl)-7-(trifluoromethyl)quinolin-4-amine. Example 18 was prepared from 4-chloro-2-(((4-phenylpiperidin-4-yl)methoxy)methyl)-7-(trifluoromethyl)quinoline by following the experimental conditions described in example 16. ¹H NMR (400 MHz, MeOD) δ ppm 8.19 (d, J=8.81 Hz, 1H), 8.04 (s, 1H), 7.57 (dd, J=8.94, 1.64 Hz, 1H), 7.38 (d, J=8.06 Hz, 2H), 7.29 (t, J=7.81 Hz, 2H), 7.16 (t, J=7.30 Hz, 1H), 6.71 (s, 1H), 4.47 (s, 2H), 3.45-3.56 (m, 2H), 2.87-3.03 (m, 8H), 2.69-2.83 (m, 2H), 2.27-2.40 (m, 2H), 1.94-2.08 (m, 2H). Mass 444 [M+H]⁺.

### Intermediate 10

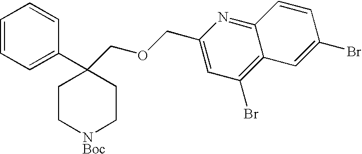

tert-butyl 4-(((4,6-dibromoquinolin-2-yl)methoxy)methyl-4-phenylpiperidine-1-carboxylate. Intermediate 10 was prepared from 4-bromoaniline by following the experimental conditions described for the synthesis of BOC-protected intermediate in example 13. ¹H NMR (400 MHz, CDCl₃) δ ppm 8.30 (d, J=2.01 Hz, 1H), 7.72-7.86 (m, 2H), 7.44 (s, 1H), 7.35-7.40 (m, 4H), 7.26-7.30 (m, 1H), 4.57 (s, 2H), 3.77 (s, 2H), 3.50 (s, 2H), 2.95-3.14 (m, 2H), 2.18-2.26 (m, 2H), 1.84-1.93 (m, 2H), 1.43 (s, 9H). Mass 591 [M+H]⁺.

### Example 19

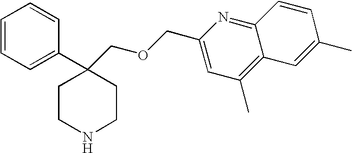

4,6-dimethyl-2-(((4-phenylpiperidin-4-yl)methoxy)methyl)quinoline. To a solution of tert-butyl 4-(((4,6-dibromoquinolin-2-yl)methoxy)methyl-4-phenylpiperidine-1-carboxylate (55 mg, 0.093 mmol) in THF (1 ml) was added trimethylboroxine (26 uL, 0.186 mmol), sodium carbonate (4M soln in water) (69 uL, 0.279 mmol), and Pd(PPh₃)₄ (5.4 mg, 0.0046 mmol). The tube was flushed with N₂, sealed, and heated at 110° C. for 2 hours in a microwave reactor. The contents were poured into ether and washed with water and brine then dried over MgSO₄ and concentrated. Column chromatography on silica gel (20-40% ethyl acetate/hexanes) afforded Boc-protected intermediate of example 19 (26 mg, 0.056 mmol, 62%). This intermediate was then treated with a trifluoroacetic acid/methylene chloride mixture (1:2, 2 ml) for 40 minutes. The solvent was removed in vacuo and the resulting crude mixture passed through a strong cation exchange column (Waters MCX, 1 g). After washing the column with several volumes of methanol, the product was eluted with 2 M ammonia in methanol to afford the title compound in example 19 (4.4 mg, 0.012 mmol, 22%). ¹H NMR (400 MHz, MeOD) δ ppm 7.70-7.78 (m, 2H), 7.49 (d, J=8.81 Hz, 1H), 7.24-7.39 (m, 4H), 7.18 (t, J=7.05 Hz, 1H), 6.92 (s, 1H), 4.40-4.52 (m, 2H), 3.41-3.48 (m, 2H), 2.80-2.95 (m, 2H), 2.59-2.73 (m, 2H), 2.41-2.55 (m, 6H), 2.21 (d, J=14.10 Hz, 2H), 1.84-2.01 (m, 2H). Mass 361 [M+H]⁺.

Table 7 describes compounds that were prepared from 3-bromoaniline following the experimental conditions described for synthesizing example 19. Retention time (t_{R}) is in min.

TABLE 7

| | | MS | HPLC | |
|---|---|---|---|---|
| Example | Structure | (MH)⁺ | (method) | ¹H NMR |
| 20 | 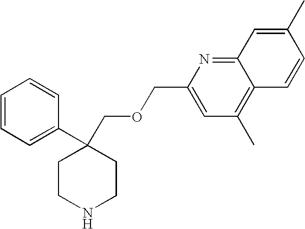 | 361.28 | 1.062(1) | ¹H NMR (400 MHz,MeOD) δ ppm 7.84-7.91(m, 1 H), 7.65 (s, 1 H),7.32-7.44 (m, 5 H), 7.22-7.28 (m, 1 H), 6.96 (s, 1H), 4.46-4.58 (m, 2 H),3.45-3.50 (m, 2 H), 3.27-3.32 (m, 2 H), 2.81-2.96 (m, 2 H), 2.55 (s, 3H), 2.42-2.50 (m, 5 H),2.22 (ddd, J = 15.1, 11.7,3.9 Hz, 2 H) |
| 21 | 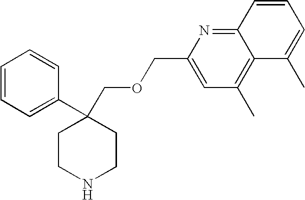 | 361.28 | 1.035(1) | ¹H NMR (400 MHz,MeOD) δ ppm 7.71 (d,J = 8.06 Hz, 1 H), 7.43-7.49 (m, 1 H), 7.31-7.42(m, 4 H), 7.18-7.31 (m, 2H), 6.93 (s, 1 H), 4.38-4.57 (m, 2 H), 3.44-3.55(m, 2 H), 3.08-3.17 (m, 2H), 2.73-2.88 (m, 8 H),2.39-2.47 (m, 2 H), 2.13(ddd, J = 15.2, 11.4, 3.4Hz, 2 H) |

### Intermediate 11

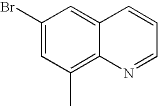

6-bromo-8-methylquinoline. Glycerol (2.3 ml, 32.2 mmol) was added dropwise via syringe to a solution of 4-bromo-2-methylaniline (5.00 g, 26.8 mmol) and sodium iodide (52 mg, 0.349 mmol) in 75% aq H₂SO₄ (15.81 ml, 120 mmol) at 140° C. The mixture was held at this temperature for 3 hours. The reaction mixture was neutralized to a pH of 7 with aq NaOH. This mixture was extracted with EtOAc and the combined organics were washed with brine, dried over MgSO₄ and concentrated. Column chromatography on silica gel (5-25% EtOAc in hexanes) afforded intermediate 11 (5.50 g, 24.7 mmol, 92%). ¹H NMR (400 MHz, CDCl₃) δ ppm 8.93 (dd, J=4.28, 1.76 Hz, 1H), 8.03 (dd, J=8.31, 1.76 Hz, 1H), 7.82 (d, J=2.01 Hz, 1H), 7.65 (d, J=1.01 Hz, 1H), 7.40 (dd, J=8.18, 4.15 Hz, 1H), 2.78 (s, 3H). Mass 221 [M+H]⁺.

### Intermediate 12

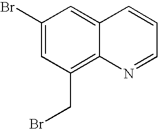

6-bromo-8-(bromomethyl)quinoline. To a solution of 6-bromo-8-methylquinoline (2.00 g, 9.00 mmol) in CCl₄ (80 ml) was added NBS (1.76 g, 9.90 mmol) and AIBN (118 mg, 0.72 mmol). The mixture was refluxed overnight under N₂ then cooled, diluted with DCM and quenched with 10% NaHSO₃. The layers were separated and the organic layer was washed with sat NaHCO₃ and brine then dried over MgSO₄ and concentrated. Column chromatography on silica gel (5-25% EtOAc in hexanes) afforded intermediate 12 (2.10 g, 6.97 mmol, 77%). ¹H NMR (400 MHz, CDCl₃) δ ppm 8.99 (dd, J=4.28, 1.76 Hz, 1H), 8.07 (dd, J=8.31, 1.51 Hz, 1H), 7.93 (dd, J=14.98, 2.14 Hz, 2H), 7.46 (dd, J=8.31, 4.03 Hz, 1H), 5.16 (s, 2H). Mass 299 [M+H]⁺.

Table 8 describes compounds that were prepared from 6-bromo-8-(bromomethyl)quinoline following the experimental conditions described in example 1, example 2, example 15, and example 19. Retention time (t_{R}) is in min.

TABLE 8

| | | MS | HPLC | |
|---|---|---|---|---|
| Example | Structure | (MH)⁺ | (method) | ¹H NMR |
| 22 | 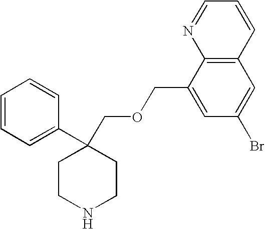 | 413.14 | 1.348(1) | ¹H NMR (400 MHz,MeOD) δ ppm 8.74(dd, J = 4.15, 1.64 Hz,1 H), 8.16 (dd,J = 8.44, 7.93 (d,J = 2.01 Hz, 1 H), 7.55(d, J = 1.26 Hz, 1 H),7.35-7.47 (m, 3 H),7.29 (t, J = 7.81 Hz, 2H), 7.15 (t, J = 7.30Hz, 1 H), 4.95 (s, 2H), 3.55 (s, 2 H), 2.82(d,J = 4.03 Hz, 2 H),2.55-2.70 (m, 2 H),2.20 (s, 2 H), 1.77-2.03 (m, 2 H), 1.64Hz, 1 H), |
| 23 | 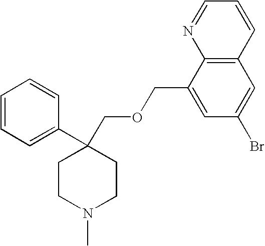 | 427.09 | 1.347(1) | ¹H NMR (400 MHz,MeOD) δ ppm 8.72-8.78 (m, 1 H), 8.11-8.20 (m, 1 H), 7.94 (t,J = 2.27 Hz, 1 H), 7.57(s, 1 H), 7.36-7.48(m, 3 H), 7.27-7.35(m, 2 H), 7.15-7.22(m, 1 H), 4.97 (d,J = 2.52 Hz, 2 H), 3.56(s, 2 H), 2.89 (s, 2 H),2.48 (s, 2 H), 2.36 (d,J = 2.52 Hz, 5 H), 2.14(s, 2 H) |
| 24 | 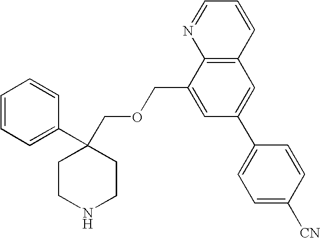 | 434.21 | 1.308(1) | ¹H NMR (400 MHz,MeOD) δ ppm 8.79(dd, J = 4.28, 1.76 Hz,1 H), 8.33 (dd,J = 8.31, 1.76 Hz, 1 H),8.08 (d, J = 2.27 Hz, 1H), 7.73-7.86 (m, 5H), 7.49 (dd, J = 8.44,4.15 Hz, 1 H), 7.41(d, J = 8.06 Hz, 2 H),7.31 (t, J = 7.68 Hz, 2H), 7.19 (t, J = 7.30Hz, 1 H), 5.08 (s, 2H), 3.61 (s, 2 H), 3.26-3.31 (m, 2 H), 2.77-2.95 (m, 2 H), 2.31-2.48 (m, 2 H), 2.05-2.24 (m, 2 H) |
| 25 | 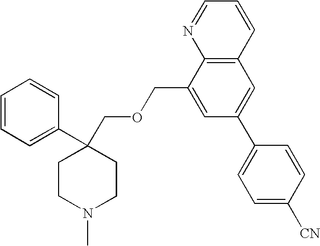 | 448.30 | 1.290(1) | ¹H NMR (400 MHz,MeOD) δ ppm 8.78(dd, J = 4.28, 1.76 Hz,1 H), 8.32 (dd,J = 8.31, 1.76 Hz, 1 H),8.05 (d, J = 2.01 Hz, 1H), 7.72-7.85 (m, 5H), 7.48 (dd, J = 8.31,4.28 Hz, 1 H), 7.39(d, J = 8.31 Hz, 2 H),7.25 (t, J = 7.81 Hz, 2H), 7.13 (t, J = 7.30Hz, 1 H), 5.06 (s, 2H), 3.61(s, 2 H), 2.76-2.86 (m, 2 H), 2.28-2.42 (m, 4 H), 2.26 (s,3 H), 2.07-2.17 (m, 2H) |
| 26 | 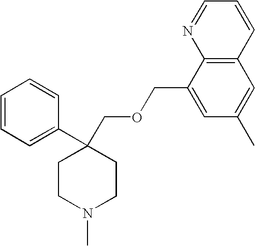 | 361.28 | 0.877(1) | ¹H NMR (400 MHz,CDCl₃) δ ppm 8.76(dd, J = 4.15, 1.64 Hz,1 H), 8.01 (dd,J = 8.31, 1.76 Hz, 1 H),7.41-7.47 (m, 3 H),7.29-7.39 (m, 4 H),7.22-7.24 (m, 1 H),5.10 (s, 2 H), 3.62 (s,2 H), 2.65-2.76 (m, 2H), 2.44 (s, 3 H), 2.10-2.40 (m, 7 H), 1.61-1.85 (m, 2 H) |
| 27 | 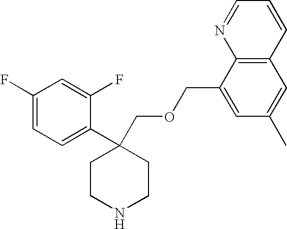 | 383.15 | 0.957(1) | ¹H NMR (500 MHz,CDCl₃) δ ppm 8.77(dd, J = 4.12, 1.68 Hz,1 H), 8.02 (dd,J = 8.24, 1.83 Hz, 1 H),7.44 (s, 1 H), 7.27-7.36 (m, 3 H), 6.81-6.89 (m, 1 H), 6.66-6.77 (m, 1 H), 5.06-5.14 (m, 2 H), 3.79 (s,2 H), 2.97-3.08 (m, 2H), 2.74-2.90 (m, 2H), 2.45 (s, 3 H), 2.29-2.36 (s, 2 H), 1.94-2.11 (m, 2 H) |
| 28 | 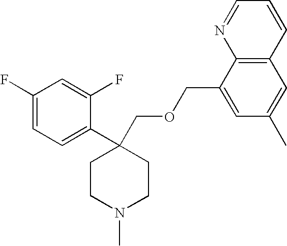 | 397.17 | 0.962(1) | ¹H NMR (400 MHz,MeOD) δ ppm 8.62(dd, J = 4.15, 1.64 Hz,1 H), 8.10 (dd,J = 8.18, 1.64 Hz, 1 H),7.48 (s, 1 H), 7.21-7.38 (m, 3 H), 6.68-6.87 (m, 2 H), 4.93 (s,2 H), 3.67 (s, 2 H),2.65-2.74 (m, 2 H),2.39-2.53 (m, 2 H),2.34 (d, J = 17.12 Hz, 8H), 2.01-2.12(m, 2 H) |
| 29 | 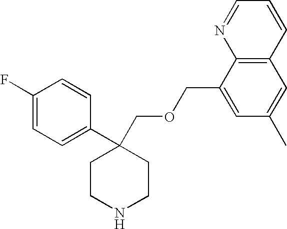 | 365.15 | 1.498(2) | ¹H NMR (400 MHz,MeOD) δ ppm 8.63(dd, J = 4.28, 1.76 Hz,1 H), 8.10 (dd,J = 8.31, 1.76 Hz, 1 H),7.47 (s, 1 H), 7.30-7.39 (m, 3 H), 7.23 (s,1 H), 6.90-7.01 (m, 2H), 4.92 (s, 2 H), 3.46-3.52 (m, 2 H), 2.75-2.88 (m, 2 H), 2.52-2.67 (m, 2 H), 2.36 (s,3 H), 2.13 (d, J = 12.09Hz, 2 H), 1.87-1.98(m, 2 H) |
| 30 | 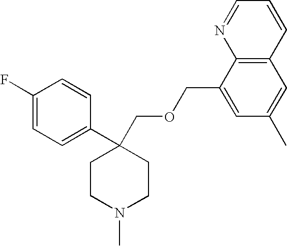 | 379.21 | 0.913(1) | ¹H NMR (400 MHz,MeOD) δ ppm 8.62(dd, J = 4.28, 1.51 Hz,1 H), 8.09 (dd,J = 8.31, 1.51 Hz, 1 H),7.46 (s, 1 H), 7.31-7.37 (m, 3 H), 7.23 (s,1 H), 6.91-6.99 (m, 2H), 4.92 (s, 2 H), 3.49(s, 2 H), 2.71-2.81(m, 2 H), 2.43 (s, 3H), 2.24-2.40 (m, 7H), 2.05-2.16 (m, 2H) |

### Example 31

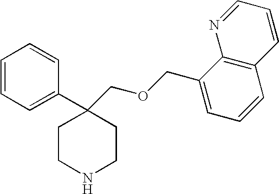

8-(((4-phenylpiperidin-4-yl)methoxy)methyl)quinoline. To a round bottomed flask under nitrogen was added tert-butyl 4-(((6-bromoquinolin-8-yl)methoxy)methyl)-4-phenylpiperidine-1-carboxylate (50 mg, 0.098 mmol) and 10% Pd/C (5 mg, 0.047 mmol) followed by ethyl acetate (0.5 ml) and methanol (0.5 ml). The flask was evacuated and backfilled with H₂ then stirred under a H₂ filled balloon overnight. The mixture was filtered through a pad of celite, rinsed with EtOAc and concentrated. Column chromatography on silica gel (20-40% EtOAc in hexanes) afforded BOC-protected intermediate of example 31 (10 mg, 0.023 mmol, 24%). This intermediate was then treated with a trifluoroacetic acid/methylene chloride mixture (1:2, 2 ml) for 40 minutes. The solvent was removed in vacuo and the resulting crude mixture was passed through a strong cation exchange column (Waters MCX, 1 g). After washing the column with several volumes of methanol, the product was eluted by washing the column with 2 M ammonia in methanol to afford the title compound in example 31 (6.1 mg, 0.018 mmol, 79%). ¹H NMR (400 MHz, MeOD) δ ppm 8.73 (dd, J=4.28, 1.51 Hz, 1H), 8.21 (dd, J=8.18, 1.39 Hz, 1H), 7.72 (d, J=7.81 Hz, 1H), 7.50 (d, J=7.05 Hz, 1H), 7.32-7.44 (m, 4H), 7.21-7.31 (m, 2H), 7.08-7.19 (m, 1H), 4.90-5.02 (m, 2H), 3.52 (s, 2H), 2.91 (s, 2H), 2.61-2.79 (m, 2H), 2.14-2.31 (m, 2H), 1.93-2.08 (m, 2H). Mass 333 [M+H]⁺.

### Intermediate 13

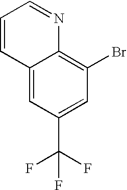

8-bromo-6-(trifluoromethyl)quinoline. To a solution of 2-bromo-4-(trifluoromethyl)aniline (3.92 g, 16.33 mmol) and sodium iodide (0.061 g, 0.408 mmol) in 75% H₂SO₄ (aq) (20 ml) at 140° C. was added glycerol (1.791 ml, 24.50 mmol) dropwise via syringe. Stirring was continued at this temperature for 3 hours, while distilling the water formed. The reaction was cooled and neutralized to pH 7 with aqueous NaOH. The mixture was extracted with EtOAc (3×100 ml). The organic portion was washed with brine, dried over MgSO₄, filtered and concentrated. The crude residue was purified by column chromatography on silica gel (15-35%) EtOAc in hexanes to afford intermediate 13 (2.57 g, 9.31 mmol, 57%). ¹H NMR (400 MHz, CDCl₃) δ ppm 9.16 (dd, J=4.15, 1.64 Hz, 1H), 8.21-8.32 (m, 2H), 8.12 (s, 1H), 7.59 (dd, J=8.31, 4.28 Hz, 1H). Mass 275 [M+H]⁺.

### Intermediate 14

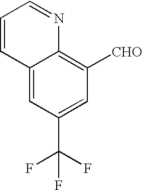

6-(trifluoromethyl)quinoline-8-carbaldehyde. To a solution of 8-bromo-6-(trifluoromethyl)quinoline (750 mg, 2.72 mmol) in diethyl ether (27 ml) cooled to −90° C., was added tBuLi (1.7 M solution in pentane) (2.88 ml, 4.89 mmol) dropwise over 10-15 minutes. The reaction mixture turned a bright yellow color upon initial addition of tBuLi and turned into a brown color when addition was complete. This mixture was stirred at −90° C. for 30 minutes then DMF (0.421 ml, 5.43 mmol) was added and the reaction was allowed to slowly warm to room temperature. After stirring at room temperature for 2 hours the reaction was quenched with water and extracted with EtOAc. The combined organics were dried over MgSO₄, filtered and concentrated. The crude residue was purified by column chromatography on silica gel (20-40%) EtOAc in hexanes to afford intermediate 14 (302 mg, 1.34 mmol, 49%). ¹H NMR (400 MHz, CDCl₃) δ ppm 11.45 (s, 1H), 9.17 (dd, J=4.03, 1.76 Hz, 1H), 8.51 (d, J=2.01 Hz, 1H), 8.31-8.43 (m, 2H), 7.63 (dd, J=8.44, 4.15 Hz, 1H). Mass 225 [M+H]⁺.

### Intermediate 15

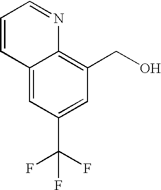

(6-(trifluoromethyl)quinolin-8-yl)methanol. A solution of 6-(trifluoromethyl)quinoline-8-carbaldehyde (80 mg, 0.355 mmol) in EtOH (3 mL) was cooled in an ice bath. Added sodium borohydride (26.9 mg, 0.711 mmol). The reaction mixture was allowed to warm to room temperature while stirring for 2 hours then quenched with a few drops of sat. NH₄Cl and concentrated to remove the EtOH. The resulting mixture was diluted with water and EtOAc. The layers were shaken and separated and the organic layer was washed with brine, dried over MgSO₄, filtered and concentrated. The crude residue was purified by column chromatography on silica gel (30-50%) EtOAc in hexanes to afford intermediate 15 (24 mg, 0.106 mmol, 29%). ¹H NMR (400 MHz, CDCl₃) δ ppm 8.99 (dd, J=4.28, 1.76 Hz, 1H), 8.30 (dd, J=8.31, 1.76 Hz, 1H), 8.09 (s, 1H), 7.79 (s, 1H), 7.56 (dd, J=8.44, 4.15 Hz, 1H), 5.24 (d, J=6.55 Hz, 2H), 4.60 (t, J=6.55 Hz, 1H). Mass 228 [M+H]⁺.

### Example 32

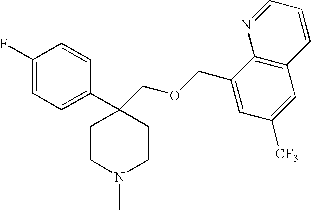

8-(((4-(4-fluorophenyl)-1-methylpiperidin-4-yl)methoxy)methyl)-6-(trifluoromethyl)quinoline. Example 32 was prepared from (6-(trifluoromethyl)quinolin-8-yl)methanol following the experimental conditions described for intermediate 1, example 1 and example 2. ¹H NMR (400 MHz, MeOD) δ ppm 8.86 (dd, J=4.15, 1.64 Hz, 1H), 8.31-8.38 (m, 1H), 8.11 (s, 1H), 7.62 (s, 1H), 7.52 (dd, J=8.31, 4.28 Hz, 1H), 7.35-7.43 (m, 2H), 6.96 (t, J=8.69 Hz, 2H), 5.01 (s, 2H), 3.49-3.64 (m, 2H), 2.74 (s, 2H), 2.30-2.48 (m, 4H), 2.34 (s, 3H) 1.95-2.16 (m, 2H). Mass 433 [M+H]⁺.

### Intermediate 16

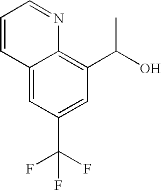

1-(6-(trifluoromethyl)quinolin-8-yl)ethanol. To a solution of 6-(trifluoromethyl)quinoline-8-carbaldehyde (302 mg, 1.341 mmol) in diethyl ether (8 ml) cooled in an ice bath, was added methyl magnesium bromide (3 M solution in diethyl ether) (0.536 ml, 1.609 mmol). The mixture was allowed to slowly warm to room temperature over one hour then quenched with sat NH₄Cl and extracted with ether. The organics were washed with brine, dried over MgSO₄, filtered and concentrated. The crude residue was purified by column chromatography on silica gel (15-35%) EtOAc in hexanes to afford intermediate 16 (266 mg, 1.10 mmol, 82%). ¹H NMR (400 MHz, CDCl₃) δ ppm 8.98 (dd, J=4.03, 1.76 Hz, 1H), 8.29 (dd, J=8.31, 1.76 Hz, 1H), 8.05 (s, 1H), 7.77 (s, 1H), 7.54 (dd, J=8.31, 4.28 Hz, 1H), 5.44-5.61 (m, 2H), 1.75 (d, J=6.55 Hz, 3H). Mass 242 [M+H]⁺.

Table 9 describes compounds that were prepared from 1-(6-(trifluoromethyl)quinolin-8-yl)ethanol following the experimental conditions described in intermediate 1, example 1 and example 2. Retention time (t_{R}) is in min.

TABLE 9

| | | MS | HPLC | |
|---|---|---|---|---|
| Example | Structure | (MH)⁺ | (method) | ¹H NMR |
| 33 | 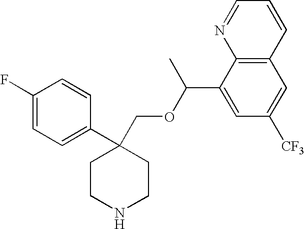 | 433.15 | 1.635(1) | ¹H NMR (400 MHz,MeOD) δ ppm 8.88 (dd,J = 4.03, 1.76 Hz, 1 H),8.34 (dd, J = 8.31, 1.76 Hz,1 H), 8.08 (s, 1 H), 7.44-7.55 (m, 2 H), 7.27-7.37(m, 2 H), 6.90-7.01 (m, 2H), 5.43-5.53 (m, 1 H),3.44 and 3.33 (AB,J_{AB} = 9.1 Hz, 2 H), 2.84-2.97 (m, 2 H), 2.62-2.76(m, 2 H), 2.22-2.33 (m, 1H), 2.12-2.22 (m, 1 H),1.83-2.04 (m, 2 H), 1.39(d, J = 6.3 Hz, 3 H) |
| 34 | 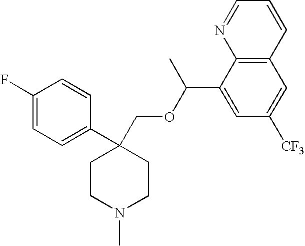 | 447.11 | 1.613(1) | ¹H NMR (400 MHz,MeOD) δ ppm 8.86 (dd,J = 4.28, 1.76 Hz, 1 H),8.33 (dd, J = 8.44, 1.64 Hz,1 H), 8.07 (s, 1 H), 7.42-7.56 (m, 2 H), 7.23-7.38(m, 2 H), 6.87-7.02 (m, 2H), 5.48 (q, J = 6.29 Hz, 1H), 3.36 (d, J = 8.81 Hz, 1H), 3.22-3.28 (m, 3 H),2.71-2.89 (m, 2 H), 2.12-2.41 (m, 5 H), 1.86-2.12 (m, 2 H), 1.31 (d,J = 6.30 Hz, 3 H) |
| 35 | 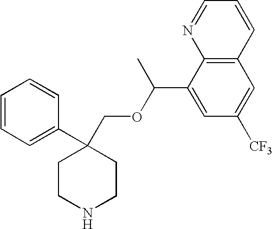 | 415.13 | 1.627(1) | ¹H NMR (400 MHz,MeOD) δ ppm 8.87 (dd,J = 4.28, 1.76 Hz, 1 H),8.34 (dd, J = 8.56, 1.76 Hz,1 H), 8.09 (s, 1 H), 7.56(d, J = 2.01 Hz, 1 H), 7.50(dd, J = 8.44, 4.15 Hz, 1H), 7.31-7.36 (m, 2 H),7.21-7.29 (m, 2 H), 7.087.17 (m, 1 H), 5.47 (q,J = 6.30 Hz, 1 H), 3.26-3.42 (m, 2 H), 2.81-2.95(m, 2 H), 2.52-2.75 (m, 2H), 1.95-2.11 (m, 2 H),1.83-2.04 (m, 2 H), 1.31(d, J = 6.55 Hz, 3 H) |
| 36 | 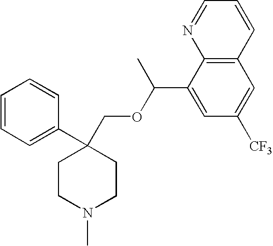 | 429.15 | 1.588(1) | ¹H NMR (400 MHz,MeOD) δ ppm, 8.86 (dd,J = 4.03, 1.76 Hz, 1 H),8.33 (dd, J = 8.31, 1.76 Hz,1 H), 8.08 (s, 1 H), 7.55(d, J = 2.01 Hz, 1 H), 7.49(dd, J = 8.44, 4.15 Hz, 1H), 7.30-7.35 (m, 2 H),7.25 (t, J = 7.68 Hz, 2 H),7.13 (t, J = 7.18 Hz, 1 H),5.48 (q, J = 6.38 Hz, 1 H),3.26-3.43 (m, 2 H), 279(s, 2 H), 2.19-2.52 (m, 7H), 1.95-2.14 (m, 2 H),1.31 (d, J = 6.30 Hz, 3 H) |
| 37 | 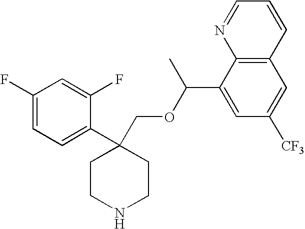 | 451.11 | 2.528(2) | ¹H NMR (400 MHz,MeOD) δ ppm 8.89 (dd,J = 4.28, 1.76 Hz, 1 H),8.35 (dd, J = 8.44, 1.64 Hz,1 H), 8.10 (s, 1 H), 7.46-7.55 (m, 2 H), 7.26-7.42(m, 1 H), 6.81-6.91 (m, 1 H),6.68-6.79 (m, 1 H),5.51 (q, J = 6.30 Hz, 1 H),3.45-3.57 (m, 2 H), 2.78-2.94 (m,2 H), 2.51-2.75 (m, 2 H), 2.29 (d,J = 2.52 Hz, 1 H), 2.19 (s,1 H), 1.75-1.99 (m, 2 H),1.29 (d, J = 6.30 Hz, 3 H) |
| 38 | 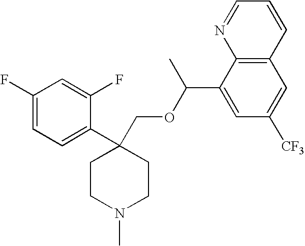 | 465.14 | 1.633(1) | ¹H NMR (400 MHz,MeOD) δ ppm 8.89 (dd,J = 4.28, 1.76 Hz, 1 H),8.35 (dd, J = 8.44, 1.64 Hz,1 H), 8.10 (s, 1 H), 7.47-7.55 (m, 2 H), 7.29-7.41(m, 1 H), 6.69-6.92 (m, 2H), 5.52 (q, J = 6.21 Hz, 1H), 3.50 (s, 2 H), 2.70-2.87 (m, 2 H), 2.32 (s, 3H), 2.26-2.55 (m, 4 H),2.00-2.18 (m, 2 H), 1.30(d, J = 6.55 Hz, 3 H) |

Table 10 describes compounds that were prepared from 8-bromo-6-methylquinoline following the experimental conditions described for the synthesis of compounds in Table 8.

TABLE 10

| | | MS | HPLC | |
|---|---|---|---|---|
| Example | Structure | [M + H]⁺ | (method) | ¹H NMR |
| 39 | 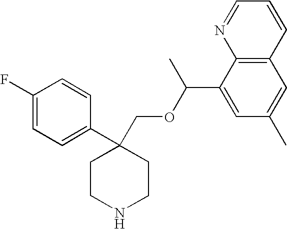 | 379.21 | 1.123(1) | ¹H NMR (400 MHz,CDCl₃) δ ppm 8.77 (dd,J = 4.28, 1.76 Hz, 1 H),8.01 (dd, J = 8.18, 1.64 Hz,1 H), 7.40 (s, 1 H), 7.29-7.36 (m, 3 H), 7.11 (d,J = 1.76 Hz, 1 H), 6.96-7.07 (m, 2 H), 5.53 (q,J = 6.13 Hz, 1 H), 3.27-3.44 (m, 2 H), 2.86-3.06(m, 2 H), 2.66-2.86 (m, 2H), 2.38 (s, 3 H), 2.17-2.26 (m, 1 H), 1.98-2.10(m, 2 H), 1.91 (ddd,J = 13.7, 9.8, 3.7 Hz, 2 H),1.40 (d, J = 6.30 Hz, 3 H) |
| 40 | 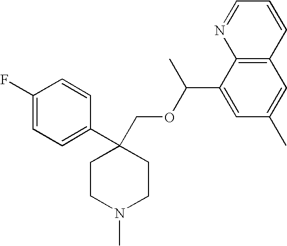 | 393.16 | 1.090(1) | ¹H NMR (400 MHz,MeOD) δ ppm 8.63 (dd,J = 4.03, 1.76 Hz, 1 H),8.08 (dd, J = 8.31, 1.76 Hz,1 H), 7.43 (s, 1 H), 7.28-7.38 (m, 3 H), 6.97-7.07(m, 3 H), 5.44 (q, J = 6.30Hz, 1 H), 3.24-3.41 (m, 3H), 2.92 (s, 2 H), 2.44-2.66 (m, 2 H), 2.40 (s, 3H), 2.28 (s, 3 H), 1.91-2.25 (m, 3 H), 1.29 (d,J = 6.30 Hz, 3 H) |
| 41 | 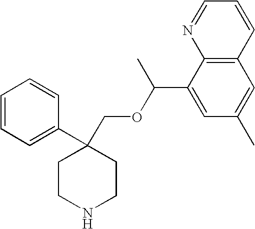 | 361.18 | 1.052(1) | ¹H NMR (400 MHz,MeOD) δ ppm 8.63 (dd,J = 4.28, 1.76 Hz, 1 H),8.07 (dd, J = 8.31, 1.76 Hz,1 H), 7.41 (s, 1 H), 7.25-7.36 (m, 5 H), 7.14-7.20(m, 1 H), 7.01 (d, J = 2.01Hz, 1 H), 5.37-5.47 (m, 1H), 3.32 (d, J = 8.81 Hz, 2H), 2.80-2.97 (m, 2 H),2.55-2.77 (m, 2 H), 2.23-2.37 (m, 4 H), 2.11 (d,J = 14.10 Hz, 1 H), 1.78-2.03 (m, 2 H), 1.28 (d,J = 6.55 Hz, 3 H) |
| 42 | 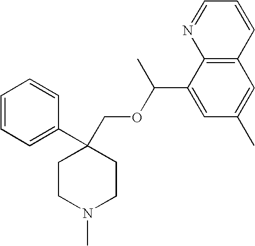 | 375.20 | 1.040(1) | ¹H NMR (500 MHz,MeOD) δ ppm 8.68 (dd,J = 4.12, 1.68 Hz, 1 H),8.12 (dd, J = 8.24, 1.53 Hz,1 H), 7.46 (s, 1 H), 7.32-7.41 (m, 5 H), 7.24 (t,J = 7.02 Hz, 1 H), 7.07 (s,1 H), 5.49 (q, J = 6.21 Hz,1 H), 3.33-3.46 (m, 2 H),2.95-3.14 (m, 2 H),2.55-2.77 (m, 2 H), 2.38 (s, 3H),2.32-2.47 (m, 1 H),2.21-2.32 (m, 1 H), 2.11-2.21 (m, 1 H), 1.34 (d,J = 6.41 Hz, 3 H) |
| 43 | 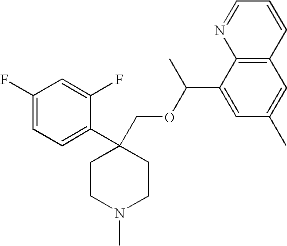 | 411.19 | 1.142(1) | ¹H NMR (400 MHz,MeOD) δ ppm 8.65 (dd,J = 4.03, 1.76 Hz, 1 H),8.09 (dd, J = 8.31, 1.76 Hz,1 H), 7.45 (s, 1 H), 7.30-7.41 (m, 2 H), 7.06 (d,J = 1.51 Hz, 1 H), 6.78-6.99 (m, 2 H), 5.48 (q,J = 6.46 Hz, 1 H), 3.41-3.52 (m, 2 H), 3.01-3.18(m, 2 H), 2.73-2.94 (m, 2H), 2.65 (s, 3 H),2.56-2.68 (m, 1 H), 2.42 (s, 3H), 2.38-2.50 (m, 1 H),2.22-2.34 (m, 1 H), 2.10-2.22 (m, 1 H), 1.23-1.33 (m, 3 H) |

### Intermediate 17

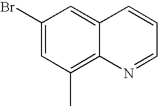

6-bromo-8-methylquinoline. Intermediate 17 was prepared from 4-bromo-2-methyl aniline by following experimental conditions described for the synthesis of intermediate 13. ¹H NMR (400 MHz, CDCl₃) δ ppm 8.93 (dd, J=4.28, 1.76 Hz, 1H), 8.03 (dd, J=8.31, 1.76 Hz, 1H), 7.82 (d, J=2.01 Hz, 1H), 7.65 (d, J=1.01 Hz, 1H), 7.40 (dd, J=8.18, 4.15 Hz, 1H), 2.78 (s, 3H). Mass 221 [M+H]⁺.

### Intermediate 18

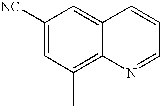

8-methylquinoline-6-carbonitrile. A solution of 6-bromo-8-methylquinoline (500 mg, 2.251 mmol) in DMF (8 ml) and water (160 μl) was degassed by bubbling through N₂ for 5 minutes. Then 1,1′-bis(diphenyphosphino)ferrocene (250 mg, 0.450 mmol) and Pd₂ dba₃ (206 mg, 0.225 mmol) were added followed by zinc cyanide (0.143 ml, 2.251 mmol). The mixture was heated at 120° C. overnight then cooled, filtered through a pad of celite and washed with EtOAc. The filtrate was washed with water and brine, dried over MgSO₄, filtered and concentrated. The crude residue was purified by column chromatography on silica gel (5-30%) EtOAc in hexanes to afford intermediate 18 (347 mg, 2.06 mmol, 92%). ¹H NMR (500 MHz, CDCl₃) δ ppm 8.99-9.11 (m, 1H), 8.16-8.22 (m, 1H), 8.07 (s, 1H), 7.70 (s, 1H), 7.52 (dd, J=8.24, 4.27 Hz, 1H), 2.83 (s, 3H). Mass 169 [M+H]⁺.

### Example 44

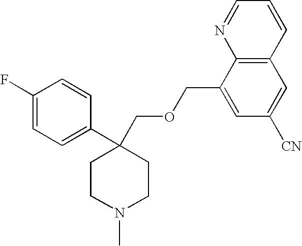

8-(((4-(4-fluorophenyl)-1-methylpiperidin-4-yl)methoxy)methyl)quinoline-6-carbonitrile. Example 44 was prepared from 8-methylquinoline-6-carbonitrile by following experimental conditions described for intermediate 3, example 1 and example 2. ¹H NMR (400 MHz, MeOD) δ ppm 8.88 (dd, J=4.28, 1.76 Hz, 1H), 8.31 (dd, J=8.31, 1.76 Hz, 1H), 8.23 (s, 1H), 7.50-7.57 (m, 2H), 7.34-7.41 (m, 2H), 6.95-7.02 (m, 2H), 4.99 (s, 2H), 3.55 (s, 2H), 2.63-2.74 (m, 2H), 2.22 (s, 3H), 2.19-2.35 (m, 4H), 2.06-2.17 (m, 2H). Mass 390 [M+H]⁺.

### Intermediate 19

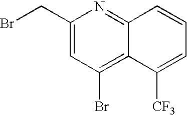

4-bromo-2-(bromomethyl)-5-(trifluoromethyl)quinoline. To a solution of (4-bromo-5-(trifluoromethyl)quinolin-2-yl)methanol (23 mg, 79 μM) in carbon tetrachloride (2.0 mL) was added N-bromosuccinimide (21 mg, 119 μM) and a catalytic amount of 2,2′-azobisisobutyronitrile. This mixture was heated at reflux for 20 hours then cooled and directly purified on silica gel eluting with hexane then 5 to 10% ethyl acetate/hexane in a linear gradient over 96 mL to yield the title compound as a white solid (10 mg, 34%). 1H NMR (400 MHz, CDCl₃) δ ppm 8.28 (d, J=8.56 Hz, 1H), 8.14 (d, J=7.55 Hz, 1H), 8.08 (s, 1H), 7.77 (t, J=7.68 Hz, 1H), 4.61 (s, 2H). Mass 737 [2M]⁺.

### Intermediate 20

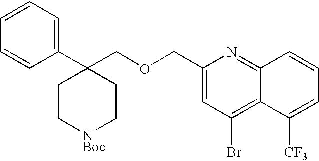

tert-butyl 4-(((4-bromo-5-(trifluoromethyl)quinolin-2-yl)methoxy)methyl)-4-phenylpiperidine-1-carboxylate. To a mixture of tert-butyl 4-(hydroxymethyl)-4-phenylpiperidine-1-carboxylate (138 mg, 0.474 mmol) and 4-bromo-2-(bromomethyl)-5-(trifluoromethyl)quinoline (175 mg, 0.474 mmol) in THF (5 ml) at ice-bath temperature was added potassium tert-butoxide (53 mg, 0.474 mmol). The mixture was stirred at this temp for 40 minutes and then additional potassium tert-butoxide (53 mg, 0.474 mmol) was added. The cooling bath was removed and the mixture was stirred for 4 hours. TLC indicated near complete reaction with small amounts of both the phenyl piperidine and quinoline methylbromide present. The mixture was quenched with sat. NH₄Cl solution and diluted with ethyl acetate and water. The layers were separated and an insoluble white suspended solid was removed by filtration before the water layer was further extracted with ethyl acetate (2×20 mL). Combined organics were washed with brine and dried over MgSO₄ then filtered and concentrated The crude material was purified on silica gel (Biotage 25S) equilibrated in hexane, loaded in CH₂Cl₂, eluted using hexane (150 mL), 22% ethyl acetate/hexane (576 mL) to yield the title compound as a clear oil (107 mg, 39%). 1H NMR (400 MHz, MeOD) δ ppm 8.08 (d, 1H), 8.00 (t, J=7.93 Hz, 1H), 8.13 (d, J=7.30 Hz, 1H), 7.45 (d, J=7.55 Hz, 2H), 7.35 (t, J=7.81 Hz, 2H), 7.20 (t, J=7.30 Hz, 1H), 6.71 (s, 1H), 4.68 (s, 2H), 3.67 (s, 2H), 3.29-3.38 (m, 2H), 3.13 (s, 3H), 2.87-2.99 (m, 2H), 2.48-2.62 (m, 2H), 2.12-2.25 (m, 2H). Mass 579 [M+H]⁺.

### Intermediate 21

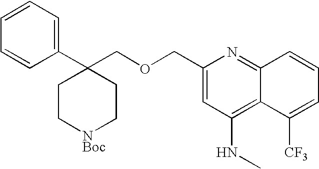

tert-butyl 4-(((4-(methylamino)-5-(trifluoromethyl)quinolin-2-yl)methoxy)methyl)-4-phenylpiperidine-1-carboxylate. To a solution of intermediate 20 (78 mg, 0.135 mmol) in absolute anhydrous Ethanol (2 ml) was added dimethylamine (650 μL, 12.83 mmol). This mixture was heated to 150° C. in a microwave reactor for 12 h. The mixture was concentrated and then dissolved in dichloromethane. Some insoluble material remained and was not soluble even upon adding small amounts of ethyl acetate. The soluble portion was separated by pipetting and was purified on silica gel (Biotage 25S), equilibrated in hexane, loaded in CH₂Cl₂, eluted using hexane (96 mL), 30% ethyl acetate/hexane (192 mL), 30 to 50% ethyl acetate/hexane (384 mL, linear gradient) to yield the title compound as a clear slightly yellow film (47.1 mg, 66%). 1H NMR (400 MHz, CDCl₃) δ ppm 8.06 (d, J=8.56 Hz, 1H), 7.80 (d, J=7.55 Hz, 1H), 7.56 (t, J=7.93 Hz, 1H), 7.30-7.42 (m, 4H), 7.21 (t, J=7.18 Hz, 1H), 6.48 (s, 1H), 5.64 (s, 1H), 4.56 (s, 2H), 3.73 (s, 2H), 3.53 (s, 2H), 3.01-3.12 (m, J=2.77 Hz, 2H), 2.87 (d, J=4.53 Hz, 3H), 2.21 (d, J=13.85 Hz, 2H), 1.86-1.98 (m, J=3.53 Hz, 2H), 1.42 (s, 9H). Mass 530 [M+H]⁺.

### Example 45

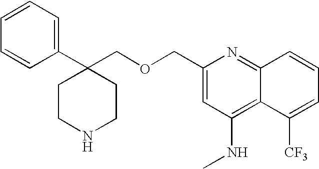

N-methyl-2-(((4-phenylpiperidin-4-yl)methoxy)methyl)-5-(trifluoromethyl)quinolin-4-amine. A solution of intermediate 21 (47.1 mg, 0.089 mmol) in dichloromethane (2 ml) was cooled in an ice-water bath and trifluoroacetic acid (200 μL, 2.60 mmol) was added. After 30 minutes the bath was removed and the mixture was allowed to come to ambient temperature over 1 hour. TLC indicated complete conversion of starting material, and the mixture was concentrated to yield the title compound (62.8 mg, 92%) based on formula mass 771.54 for 3TFA salt. 1H NMR (400 MHz, MeOD) δ ppm 8.13 (d, J=7.30 Hz, 1H), 8.08 (d, 1H), 8.00 (t, J=7.93 Hz, 1H), 7.45 (d, J=7.55 Hz, 2H), 7.35 (t, J=7.81 Hz, 2H), 7.20 (t, J=7.30 Hz, 1H), 6.71 (s, 1H), 4.68 (s, 2H), 3.67 (s, 2H), 3.29-3.38 (m, 2H), 3.13 (s, 3H), 2.87-2.99 (m, 2H), 2.48-2.62 (m, 2H), 2.12-2.25 (m, J=3.78 Hz, 2H). Mass 430 [M+H]⁺.

### Intermediate 22

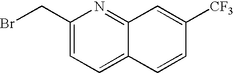

2-(bromomethyl)-7-(trifluoromethyl)quinoline. This compound was prepared according to the procedure for Intermediate 19 using 2-methyl-7-(trifluoromethyl)quinoline (200 mg, 0.95 mmol), carbon tetrachloride (21 mL), N-bromosuccinimide (233 mg, 1.31 mmol) and 2,2′-azobisisobutyronitrile (20 mg, 0.122 mmol) to yield the title compound as a white solid (103 mg, 37%). 1H NMR (400 MHz, CDCl₃) δ ppm 8.36 (s, 1H), 8.20 (d, J=8.56 Hz, 1H), 7.91 (d, J=8.56 Hz, 1H), 7.70 (dd, J=8.56, 1.51 Hz, 1H), 7.66 (d, J=8.56 Hz, 1H), 4.69 (s, 2H). Mass 290 [M+H]⁺.

### Intermediate 23

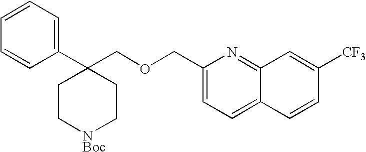

tert-butyl 4-phenyl-4-(((7-(trifluoromethyl)quinolin-2-yl)methoxy)methyl)piperidine-1-carboxylate. This compound was prepared according to the procedure for Intermediate 20 using Intermediate 22 (99.8 mg, 0.344 mmol), tert-butyl 4-(hydroxymethyl)-4-phenylpiperidine-1-carboxylate (91 mg, 0.31 mmol), potassium tert-butoxide (70.0 mg, 0.624 mmol) in THF (3.0 mL) over 3 hours to yield the title compound as a clear film (84 mg, 54%). 1H NMR (400 MHz, CDCl₃) δ ppm 8.29 (s, 1H), 8.07 (d, J=8.56 Hz, 1H), 7.89 (d, J=8.31 Hz, 1H), 7.66 (d, J=8.56 Hz, 1H), 7.21-7.39 (m, 6H), 4.66 (s, 2H), 3.76 (s, 2H), 3.49 (s, 2H), 3.04 (s, 2H), 2.21 (d, J=13.85 Hz, 2H), 1.85-1.98 (m, 2H), 1.42 (s, 9H). Mass 501 [M+H]⁺.

### Example 46

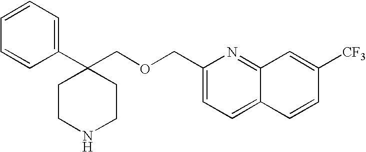

2-(((4-phenylpiperidin-4-yl)methoxy)methyl)-7-(trifluoromethyl)quinoline. This compound was prepared according to the procedure for Example 45 using Intermediate 23 (84 mg, 0.168 mmol) and trifluoroacetic acid (1.0 mL) in dichloromethane (5.0 mL) for 1.5 hours. The product was converted to the free base by passage through an ion exchange cartridge (Waters MCX, 1 g) eluting with 2M ammonia in methanol then purified on silica gel eluting with 2% 2M ammonia in methanol/dichloromethane to yield the title compound as a clear film (51.6 mg, 77%). 1H NMR (400 MHz, MeOD) δ ppm 8.24 (d, J=8.6 Hz, 1H), 8.18-8.21 (m, 1H), 8.04 (d, J=8.6 Hz, 1H), 7.72 (dd, J=8.3, 1.5 Hz, 1H), 7.28-7.46 (m, 5H), 7.20 (t, J=7.3 Hz, 1H), 4.60 (s, 2H), 3.53 (s, 2H), 2.86 (td, J=8.6, 3.9 Hz, 2H), 2.61-2.72 (m, 2H), 2.18-2.29 (m, 2H), 1.88-2.00 (m, 2H). Mass 401 [M+H]⁺.

### Intermediate 24

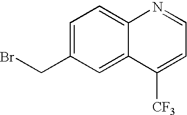

6-(bromomethyl)-4-(trifluoromethyl)quinoline. This compound was prepared according to the procedure for Intermediate 19 using 4-(trifluoromethyl)-6-methyl-quinoline (203 mg, 0.961 mmol), carbon tetrachloride (20 mL), N-bromosuccinimide (257 mg, 1.44 mmol) and 2,2′-azobisisobutyronitrile (20 mg, 0.122 mmol). Purification on silica gel eluting with a hexane-ethyl acetate gradient provided the title compound as a white solid (62.1 mg, 22%). 1H NMR (400 MHz, CDCl₃) δ ppm 8.97-9.04 (d, J=4.28 Hz, 1H), 8.17 (d, J=8.81 Hz, 1H), 8.06 (s, 1H), 7.82 (d, J=8.81 Hz, 1H), 7.67 (d, J=4.28 Hz, 1H), 4.65 (s, 2H). Mass 290 [M+H]⁺.

### Intermediate 25

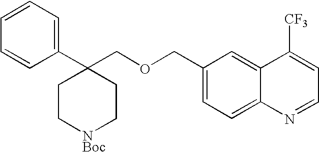

tert-butyl 4-phenyl-4-(((4-(trifluoromethyl)quinolin-6-yl)methoxy)methyl)piperidine-1-carboxylate. This compound was prepared according to the procedure for Intermediate 20 using Intermediate 24 (56.2 mg, 0.195 mmol), tert-butyl 4-(hydroxymethyl)-4-phenylpiperidine-1-carboxylate (56.9 mg, 0.195 mmol), potassium tert-butoxide (42.0 mg, 0.374 mmol) in THF (1.5 mL) over 20 hours to yield the title compound as a clear film (38.6 mg, 40%). 1H NMR (400 MHz, CDCl₃) δ ppm 8.98 (d, J=4.53 Hz, 1H), 8.09 (d, J=8.81 Hz, 1H), 7.95 (s, 1H), 7.67 (d, J=4.53 Hz, 1H), 7.53 (dd, J=8.81, 1.51 Hz, 1H), 7.31-7.40 (m, 4H), 7.20-7.26 (m, 1H), 4.56 (s, 2H), 3.67-3.84 (m, 2H), 3.46 (s, 2H), 3.03 (app. t, J=11.21 Hz, 2H), 2.21 (d, J=13.85 Hz, 2H), 1.85-1.95 (m, J=3.78 Hz, 2H), 1.42 (s, 9H). Mass 501 [M+H]⁺.

### Example 47

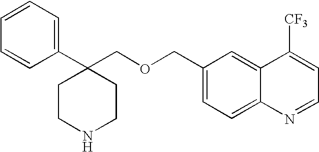

6-(((4-phenylpiperidin-4-yl)methoxy)methyl)-4-(trifluoromethyl)quinoline. This compound was prepared according to the procedure for Example 45 using Intermediate XVI (38.6 mg, 0.077 mmol) and trifluoroacetic acid (0.2 mL) in dichloromethane (2.0 mL) for 2.5 hours. The product was converted to the free base by passage through an ion exchange cartridge (Waters MCX, 1 g) eluting with 2M ammonia in methanol then purified on silica gel eluting with 2% 2M ammonia in methanol/dichloromethane to yield the title compound as a clear film (26.7 mg, 86%). 1H NMR (400 MHz, MeOD) δ ppm 8.36 (d, J=8.81 Hz, 1H), 8.14 (d, J=8.31 Hz, 1H), 7.93 (d, J=7.30 Hz, 1H), 7.77 (t, J=7.93 Hz, 1H), 7.26-7.43 (m, 5H), 7.20 (t, J=7.30 Hz, 1H), 4.59 (s, 2H), 3.52 (s, 2H), 2.82-2.92 (m, 2H), 2.59-2.72 (m, 2H), 2.17-2.28 (m, 2H), 1.87-2.01 (m, 2H). Mass 401 [M+H]⁺.

### Intermediate 26

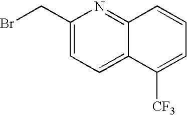

2-(bromomethyl)-5-(trifluoromethyl)quinoline. This compound was prepared according to the procedure for Intermediate 19 using 2-methyl-5-(trifluoromethyl)quinoline (200 mg, 0.947 mmol), carbon tetrachloride (20 mL), N-bromosuccinimide (243 mg, 1.36 mmol) and 2,2′-azobisisobutyronitrile (20 mg, 0.122 mmol). Purification on silica gel eluting with a hexane-ethyl acetate gradient provided the title compound (103 mg, 37%) as a white solid. 1H NMR (400 MHz, CDCl₃) δ ppm 8.47-8.54 (m, 1H), 8.24 (d, J=8.56 Hz, 1H), 7.92 (d, J=7.30 Hz, 1H), 7.76 (t, J=7.93 Hz, 1H), 7.69 (d, J=9.06 Hz, 1H), 4.70 (s, 2H). Mass 290 [M+H]⁺.

### Intermediate 27

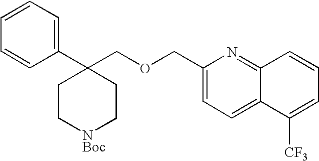

tert-butyl 4-phenyl-4-(((5-(trifluoromethyl)quinolin-2-yl)methoxy)methyl)piperidine-1-carboxylate. This compound was prepared according to the procedure for Intermediate 20 using Intermediate 26 (51.3 mg, 0.177 mmol), tert-butyl 4-(hydroxymethyl)-4-phenylpiperidine-1-carboxylate (50.3 mg, 0.173 mmol), potassium tert-butoxide (38.8 mg, 0.346 mmol) in THF (2.0 mL) over 21 hours to yield the title compound as a clear oil (56.8 mg, 82%). 1H NMR (400 MHz, CDCl₃) δ ppm 8.35 (d, J=8.81 Hz, 1H), 8.16 (d, J=8.56 Hz, 1H), 7.86 (d, J=7.30 Hz, 1H), 7.69 (t, J=7.93 Hz, 1H), 7.28-7.39 (m, 5H), 7.22-7.27 (m, 1H), 4.65 (s, 2H), 3.76 (s, 2H), 3.50 (s, 2H), 3.04 (t, J=11.21 Hz, 2H), 2.21 (d, J=13.85 Hz, 2H), 1.85-1.97 (m, 2H), 1.42 (s, 9H). Mass 501 [M+H]⁺.

### Example 48

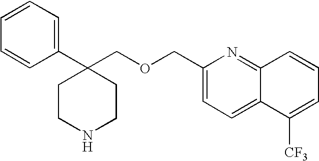

2-(((4-phenylpiperidin-4-yl)methoxy)methyl)-5-(trifluoromethyl)quinoline. This compound was prepared according to the procedure for Example 45 using Intermediate 27 (57 mg, 0.114 mmol) and trifluoroacetic acid (0.2 mL) in dichloromethane (3.0 mL) for 2.5 hours. The product was converted to the free base by passage through an ion exchange cartridge (Waters MCX, 1 g) eluting with 2M ammonia in methanol then purified on silica gel eluting with 2% 2M ammonia in methanol/dichloromethane to yield the title compound as a yellow film (41.9 mg, 92%). 1H NMR (400 MHz, MeOD) δ ppm 8.36 (d, J=8.81 Hz, 1H), 8.14 (d, J=8.31 Hz, 1H), 7.93 (d, J=7.30 Hz, 1H), 7.77 (t, J=7.93 Hz, 1H), 7.34-7.43 (m, 3H), 7.31 (t, J=7.81 Hz, 2H), 7.20 (t, J=7.30 Hz, 1H), 4.59 (s, 2H), 3.52 (s, 2H), 2.83-2.93 (m, 2H), 2.62-2.73 (m, 2H), 2.23 (d, J=14.1 Hz, 2H), 1.94 (ddd, J=13.8, 10.4, 3.7 Hz, 2H). Mass 401 [M+H]⁺.

### Intermediate 28

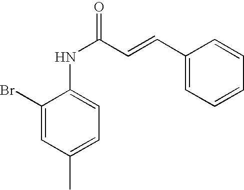

Potassium carbonate (20.4 g, 148 mmol) was dissolved in water (100 mL) at room temperature, cooled to 0° C., and treated with ice (150 mL), acetone (200 mL), and 2-bromo-4-methylaniline (25.0 g, 134 mmol). To this was slowly added cinnamoyl chloride (23.5 g, 141 mmol) as a solution in acetone (100 mL). After 1 h, the mixture was poured into ice water. The product was collected by filtration to give 39.8 g (94%) as a white powder. 1H-NMR (CDCl₃, 500 MHz) δ 8.34 (bs, 1H), 7.76 (d, J=15.6 Hz, 1H), 7.74 (s, 1H), 7.56 (m, 2H), 7.38 (m, 4H), 7.13 (d, J=8.2 Hz, 1H), 6.58 (d, J=15.6 Hz, 1H), 2.30 (s, 3H); ¹³C-NMR (126 MHz, CDCl₃) δ ppm 163.8, 142.8, 135.5, 134.7, 133.5, 132.6, 130.2, 129.2, 129.0, 128.1, 122.1, 120.9, 20.6.

### Intermediate 29

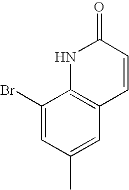

To a suspension of N-(2-bromo-4-methylphenyl)cinnamamide (39.8 g, 126 mmol) in chlorobenzene (200 mL, 126 mmol) at 0° C. was added aluminum trichloride (84 g, 629 mmol). The ice bath was removed and the reaction warmed to reflux. After heating at reflux for 1 h, the reaction was cooled to room temperature, poured onto ice (500 mL), and diluted with methanol (400 mL). The resulting mixture was stirred at room temperature for 1 h and transferred to a separatory funnel. The mixture was extracted with dichloromethane (5×). The combined organic layers were dried over magnesium sulfate, and concentrated. The resulting solid was suspended in diethyl ether and filtered to give 17.4 g (58%) as a light pink solid. HNMR and LC/MS show that it is a 3.5:1 mixture of product:debrominated product. Used without purification. ¹H-NMR (CDCl₃, 500 MHz) δ 9.03 (bs, 1H), 7.61 (d, J=9.5 Hz, 1H), 7.52 (s, 1H), 7.27 (s, 1H), 6.63 (d, J=9.5 Hz, 1H), 2.37 (s, 3H); ¹³C-NMR (126 MHz, CDCl₃) δ ppm 162.2, 140.2, 134.7, 133.3, 127.5, 123.0. Mass spec.: 237.82 (MH)⁺.

### Intermediate 30

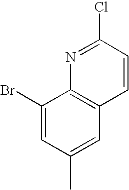

A flask was charged with 8-bromo-6-methylquinolin-2(1H)-one (17.4 g, 73.1 mmol) and phosphorous oxychloride (170 mL, 1.82 mol). The resulting suspension was heated to reflux and held there for 2 h. The reaction was concentrated, diluted with dichloromethane, poured onto ice, and made basic by addition of aqueous ammonia. The mixture was extracted with dichloromethane (2×). The organics were washed with brine, dried over magnesium sulfate, and concentrated. The resulting amorphous solid was dissolved in hot ethanol (200 mL), allowed to cool to room temperature, and placed in a 0° C. bath for 1 h. The resulting precipitate was collected by filtration, washed with a minimum of cold ethanol, and air dried to give 12.6 g (67%) as fine needles. ¹H-NMR (CDCl₃, 500 MHz) δ 7.99 (d, J=8.6 Hz, 1H), 7.90 (d, J=1.8 Hz, 1H), 7.52 (s, 1H), 7.38 (d, J=8.5 Hz, 1H), 2.50 (s, 3H); ¹³C-NMR (126 MHz, CDCl₃) δ ppm 151.1, 143.8, 138.8, 137.9, 136.3, 128.1, 126.5, 123.5, 123.2, 21.3. Mass spec.: 256.01 (MH)⁺.

### Intermediate 31

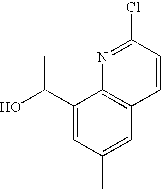

To a solution of 8-bromo-2-chloro-6-methylquinoline (2.5 g, 9.75 mmol) in tetrahydrofuran (25 mL) at −78° C. was added tert-butyllithium (1.7 M in pentane, 6.88 mL, 11.7 mmol) dropwise. After 10 min, the reaction was treated with acetaldehyde (1.1 mL, 19.5 mmol). The ice bath was removed and the reaction allowed to warm to room temperature over 45 min. The reaction was quenched by addition of saturated ammonium chloride, and diluted with diethyl ether. The ethereal was washed with water (2×), then brine, dried over magnesium sulfate, and concentrated. Column chromatography (8%→20% EtOAc/Hex) gave 1.46 g (68%). ¹H-NMR (CDCl₃, 500 MHz) δ 7.98 (d, J=8.5 Hz, 1H), 7.48 (s, 1H), 7.44 (s, 1H), 7.31 (d, J=8.5 Hz, 1H), 5.46 (m, 1H), 4.66 (d, J=6.4 Hz, 1H), 2.49 (s, 3H), 1.68 (d, J=6.7 Hz, 3H); ¹³C-NMR (126 MHz, CDCl₃) δ ppm 148.4, 144.4, 141.5, 138.9, 136.9, 129.6, 127.4, 125.7, 122.2, 68.8, 24.0, 21.7. Mass spec.: 222.13 (MH)⁺.

### Intermediate 32

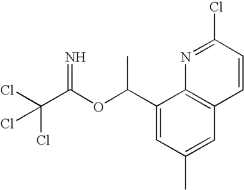

To a solution of 1-(2-chloro-6-methylquinolin-8-yl)ethanol (2.5 g, 11.3 mmol) and trichloroacetonitrile (11.3 mL, 113 mmol) in dichloromethane (80 mL) at room temperature was added diazabicycloundecene (0.17 mL, 1.13 mmol). The reaction was stirred at room temperature for 4 h and concentrated to give a moist solid. The crude product was transferred to an ehrlenmeyer flask, and treated with 225 mL of diethyl ether. The mixture was vigorously stirred for 10 min. The resulting mixture was filtered through a plug of cotton and the ethereal concentrated. The resulting solid was dissolved in diethyl ether again (225 mL), stirred 10 min, filtered through a plug of cotton, and the etheral concentrated to give 4.07 g (99%) as a light yellow solid. ¹H-NMR (CDCl₃, 500 MHz) δ 8.31 (s, 1H), 7.99 (d, J=8.5 Hz, 1H), 7.73 (s, 1H), 7.50 (s, 1H), 7.34 (d, J=8.5 Hz, 1H), 7.04 (q, J=6.4 Hz, 1H), 2.51 (s, 3H), 1.76 (d, J=6.4 Hz, 3H).

### Intermediate 33

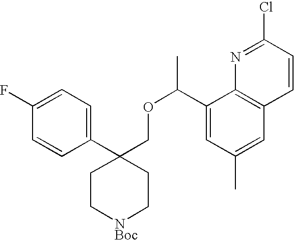

To a solution of 1-(2-chloro-6-methylquinolin-8-yl)ethyl 2,2,2-trichloroacetimidate (4.02 g, 11.0 mmol) and tert-butyl 4-(4-fluorophenyl)-4-(hydroxymethyl)piperidine-1-carboxylate (3.74 g, 12.1 mmol) in dichloromethane (25 mL) at 0° C. was added cyclohexane (25 mL) and tetrafluoroboric acid diethyletherate (0.075 mL, 0.55 mmol). The reaction was stirred at 0° C. for 10 min. The reaction was treated with an additional portion of tetrafluoroboric acid diethyletherate (0.050 mL). After 30 min at 0° C., The reaction was quenched by addition of saturated sodium bicarbonate, diluted with diethyl ether, and poured into water. The ethereal was washed with water (2×), then brine, dried over magnesium sulfate, and concentrated. Column chromatography (12%→18% EtOAc/Hex) gave 2.56 g (45%) as an amorphous foam solid. ¹H-NMR (CDCl₃, 500 MHz) δ 7.94 (d, J=8.5 Hz, 1H), 7.39 (s, 1H), 7.24-7.34 (m, 3H), 7.11 (s, 1H), 7.03 (m, 2H), 5.45 (q, J=6.4 Hz, 1H), 3.68 (m, 2H), 3.33 (q_{AB}, J_{AB}=9.2, 2H), 3.08 (m, 1H), 3.06 (m, 1H), 2.37 (s, 3H), 2.20 (m, 1H), 1.90-2.10 (m, 2H), 1.85 (m, 1H), 1.44 (s, 9H), 1.38 (d, J=6.4 Hz, 3H). Mass spec.: 513.69 (MH)⁺.

### Intermediate 34

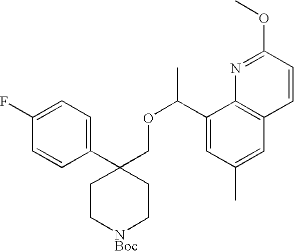

A microwave tube was charged with tert-butyl 4-((1-(2-chloro-6-methylquinolin-8-yl)ethoxy)methyl)-4-(4-fluorophenyl)piperidine-1-carboxylate (100 mg, 0.195 mmol), sodium methoxide (42.1 mg, 0.78 mmol), and methanol (3 mL). The tube was purged with nitrogen, sealed and heated at 85° C. for 2 h. The reaction was treated with an additional portion of sodium methoxide (42.1 mg, 0.78 mmol) and heated at 130° C. for 6 h. The reaction was concentrated, diluted with diethyl ether, washed with water (2×), then brine, dried over magnesium sulfate, and concentrated. Column chromatography (EtOAc/Hex) gave 58 mg (59%) as a white foam. ¹H-NMR (CDCl₃, 500 MHz) δ 7.84 (d, J=8.9 Hz, 1H), 7.28-7.37 (m, 3H), 6.98-7.08 (m, 3H), 6.82 (d, J=8.5 Hz, 1H), 5.35 (q, J=6.4 Hz, 1H), 3.97 (s, 3H), 3.71 (m, 2H), 3.33 (s, 2H), 3.07 (m, 2H), 2.35 (s, 3H), 2.20 (m, 1H), 2.08 (m, 1H), 1.96 (m, 1H), 1.88 (m, 1H), 1.44 (s, 9H), 1.40 (d, J=6.4 Hz, 3H); ¹³C-NMR (126 MHz, CDCl₃) δ ppm 162.4, 160.9, 160.4, 155.1, 142.0, 140.0, 139.3 (m), 138.5, 133.5, 129.1, 129.0, 127.4, 125.4, 124.8, 115.1, 114.9, 112.5, 79.4, 77.7, 73.5, 53.2, 41.3, 40.4 (br), 32.2 (br), 28.6, 23.1, 22.8, 21.6, 14.2. Mass spec.: 509.37 (MH)⁺.

### Intermediate 35

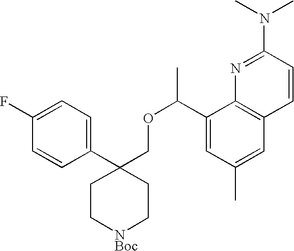

tert-Butyl 4-((1-(2-chloro-6-methylquinolin-8-yl)ethoxy)methyl)-4-(4-fluorophenyl)piperidine-1-carboxylate (100 mg, 0.195 mmol) was dissolved in dimethylamine (33% in ethanol, 2.5 mL) and heated via microwave at 120° C. for 2 h. The reaction was concentrated and the residue loaded directly onto a silica gel column. Column chomatography (8%→16% EtOAc/Hex) gave 88.2 mg (87%) as a white foam solid. ¹H-NMR (CDCl₃, 500 MHz) δ 7.74 (d, J=8.9 Hz, 1H), 7.34 (dd, J=8.5 Hz, 5.2 Hz, 2H), 7.21 (s, 1H), 7.03 (m, 2H), 7.00 (d, J=1.5 Hz, 1H), 6.82 (d, J=9.2 Hz, 1H), 5.36 (q, J=6.4 Hz, 1H), 3.71 (m, 2H), 3.34 (q_{AB}, J_{AB}=9.2 Hz, 2H), 3.15 (s, 6H), 3.03-3.20 (m, 2H), 2.33 (s, 3H), 2.18 (m, 1H), 2.09 (m, 1H), 1.98 (m, 1H), 1.89 (m, 1H), 1.46 (s, 9H), 1.40 (d, J=6.4 Hz, 3H); ¹³C-NMR (126 MHz, CDCl₃) δ ppm 162.4, 160.4, 156.3, 155.1, 143.7, 139.5 (m), 138.9, 136.9, 130.8, 129.1, 129.0, 127.2, 125.1, 122.1, 115.0, 114.9, 108.6, 79.4, 77.5, 73.7, 41.3, 40.3 (br), 38.1, 32.1 (br), 28.6, 22.9, 21.4, 14.2. Mass spec.: 522.69 (MH)⁺.

### Intermediate 36

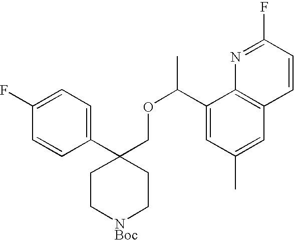

A microwave tube was charged with tert-butyl 4-((1-(2-chloro-6-methylquinolin-8-yl)ethoxy)methyl)-4-(4-fluorophenyl)piperidine-1-carboxylate (100 mg, 0.195 mmol), potassium fluoride (56.6 mg, 0.975 mmol), kryptofix 222 (110 mg, 0.292 mmol), and dimethylsulfoxide (3 mL). The tube was purged with nitrogen, sealed, and heated at 130° C. for 10 h. The reaction was treated with an additional portion of kryptofix 222 (110 mg, 0.292 mmol) and potassium fluoride (56.6 mg, 0.975 mmol) and heated at 140° C. for 2 h. The reaction was poured into diethyl ether/water. The ethereal was washed with water (2×), then brine, dried over magnesium sulfate, and concentrated. Column chromatography (8%→12% EtOAc/Hex) gave 51 mg (53%) as a colorless film. ¹H-NMR (CDCl₃, 500 MHz) δ 8.09 (m, 1H), 7.43 (s, 1H), 7.31 (dd, J=8.9 Hz, 5.2 Hz, 2H), 7.12 (s, 1H), 7.02 (m, 2H), 6.99 (dd, J=8.9 Hz, 2.8 Hz, 1H), 5.36 (q, J=6.4 Hz, 1H), 3.68 (m, 2H), 3.32 (q_{AB}, J_{AB}=8.9 Hz, 2H), 3.11 (m, 1H), 3.04 (m, 1H), 2.38 (s, 3H), 2.21 (m, 1H), 2.00 (m, 2H), 1.84 (m, 1H), 1.44 (s, 9H), 1.37 (d, J=6.1 Hz, 3H); ¹³C-NMR (126 MHz, CDCl₃) δ ppm 162.4, 161.0, 160.4, 159.1, 155.1, 141.6, 141.5, 141.2, 139.3 (m), 136.0 (m), 129.0, 128.9, 128.8, 126.9, 125.4, 115.1, 114.9, 109.8, 109.4, 79.4, 77.6, 72.9, 41.3, 40.2 (br), 32.0 (br), 28.6, 23.5, 21.6. Mass spec.: 497.43 (MH)⁺.

### Intermediate 37

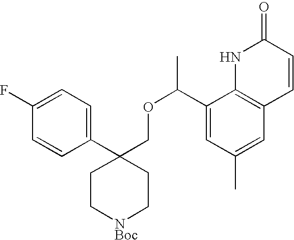

A microwave tube was charged with tert-butyl 4-((1-(2-chloro-6-methylquinolin-8-yl)ethoxy)methyl)-4-(4-fluorophenyl)piperidine-1-carboxylate (100 mg, 0.195 mmol), potassium hydroxide (crushed with a mortar and pestle, 87 mg, 1.56 mmol), kryptofix 222 (110 mg, 0.292 mmol), and tert-butanol (3 mL). The tube was purged with nitrogen, sealed, and heated at 130° C. for 2 h. The reaction was concentrated, quenched by addition of saturated ammonium chloride, and diluted with diethyl ether. The ethereal was washed with water (2×), then brine, dried over magnesium sulfate, and concentrated. Column chromatography (37%→60% EtOAc/Hex) gave 76.6 mg (79%) as a viscous oil. ¹H-NMR (CDCl₃, 500 MHz) δ 9.80 (bs, 1H), 7.62 (d, J=9.8 Hz, 1H), 7.15-7.35 (m, 3H), 6.98 (m, 2H), 6.88 (d, J=1.2 Hz, 1H), 6.57 (dd, J=9.5 Hz, 1.2 Hz, 1H), 4.34 (bs, 1H), 3.74 (m, 2H), 3.32 (m, 1H), 3.27 (m, 1H), 2.94 (m, 2H), 2.31 (s, 3H), 2.16 (m, 1H), 2.11 (m, 1H), 1.81 (m, 2H), 1.40 (m, 12H); ¹³C-NMR (126 MHz, CDCl₃) δ ppm 162.5, 161.9, 160.5, 155.0, 140.5, 137.9, 134.1, 131.4, 130.6, 128.81, 128.75, 127.5, 126.7, 122.2, 120.6, 115.5, 115.4, 80.2, 79.5, 79.0, 41.3, 40.0 (br), 32.2, 31.9, 28.5, 21.6, 20.7. Mass spec.: 495.44 (MH)⁺.

### Example 49

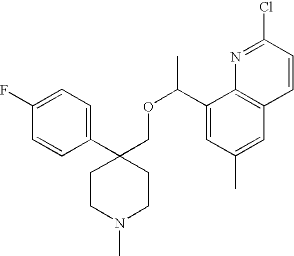

tert-Butyl 4-((1-(2-chloro-6-methylquinolin-8-yl)ethoxy)methyl)-4-(4-fluorophenyl)piperidine-1-carboxylate (100 mg, 0.195 mmol) was dissolved in trifluoroacetic acid (20% in dichloromethane, 1 mL), stirred for 30 min at room temperature, and concentrated. The resulting residue was dissolved in methanol, loaded onto a strong cation exchange cartridge, and washed with several volumes of methanol which were discarded. The crude product was eluted with 2M ammonia in methanol and concentrated. The crude product was dissolved in acetonitrile (1.5 mL) and treated with sodium cyanoborohydride (24.5 mg, 0.39 mmol) and formalin (100 μL). To this was added 1 drop of acetic acid. After 1 h, the reaction was concentrated, diluted with diethyl ether, washed with 1 M sodium hydroxide, then water, then brine, dried over magnesium sulfate, and concentrated. Column chomatography (5% methanol/dichloromethane→10% 2M ammonia in methanol/dichloromethane) gave 46 mg (55%). ¹H-NMR (CDCl₃, 500 MHz) δ 7.93 (d, J=8.5 Hz, 1H), 7.38 (s, 1H), 7.32 (dd, J=8.9 Hz, 5.5 Hz, 2H), 7.27 (d, J=8.5 Hz, 1H), 7.10 (d, J=1.5 Hz, 1H), 7.02 (m, 2H), 5.43 (q, J=6.4 Hz, 1H), 3.41 (d, J=8.9 Hz, 1H), 3.25 (d, J=8.9 Hz, 1H), 2.59 (m, 2H), 2.36 (s, 3H), 2.23 (s, 3H), 2.16-2.32 (m, 3H), 2.12 (m, 2H), 1.99 (m, 1H), 1.37 (d, J=6.4 Hz, 3H); ¹³C-NMR (126 MHz, CDCl₃) δ ppm 162.3, 160.3, 148.8, 144.0, 141.6, 140.3 (br), 138.4, 137.0, 129.0, 128.8, 126.9, 125.3, 122.0, 114.9, 114.7, 72.8, 52.02, 51.96, 46.2, 40.3, 32.7, 32.2, 23.6, 21.7. Mass spec.: 427.49 (MH)⁺.

### Example 50

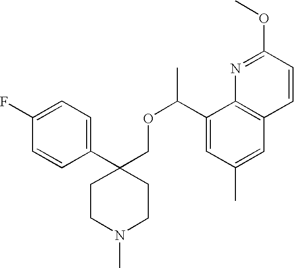

¹H-NMR (CDCl₃, 500 MHz) δ 7.84 (d, J=8.9 Hz, 1H), 7.34 (dd, J=8.9 Hz, 5.5 Hz, 2H), 6.97-7.08 (m, 3H), 6.81 (d, J=8.9 Hz, 1H), 5.33 (q, J=6.4 Hz, 1H), 3.96 (s, 3H), 3.32 (s, 2H), 2.59 (m, 2H), 2.34 (s, 3H), 2.22 (s, 3H), 1.95-2.30 (m, 7H), 1.38 (d, J=6.4 Hz, 3H); ¹³C-NMR (126 MHz, CDCl₃) δ ppm 162.2, 160.9, 160.3, 142.0, 140.1, 138.4, 133.5, 129.1 (m), 127.5, 125.3, 124.8, 114.9, 114.7, 112.4, 73.4, 53.2, 52.0 (m), 46.3, 40.5, 32.7, 32.3, 23.1, 21.5. Mass spec.: 423.44 (MH)⁺.

### Example 51

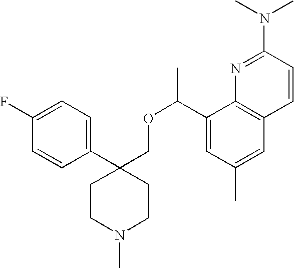

¹H-NMR (CDCl₃, 500 MHz) δ 7.74 (d, J=9.2 Hz, 1H), 7.33 (dd, J=8.7 Hz, 5.3 Hz, 2H), 7.20 (s, 1H), 7.03 (m, 2H), 6.96 (d, J=1.5 Hz, 1H), 6.82 (d, J=8.9 Hz, 1H), 5.34 (q, J=6.2 Hz, 1H), 3.34 (q_{AB}, J_{AB}=9.2 Hz, 2H), 3.14 (s, 6H), 2.72 (m, 2H), 2.23-2.40 (m, 9H), 2.02-2.23 (m, 3H), 1.38 (d, J=6.4 Hz, 3H); ¹³C-NMR (126 MHz, CDCl₃) δ ppm 162.3, 160.4, 156.3, 143.7, 138.9, 136.9, 130.8, 129.03, 128.97, 127.2, 125.1, 122.1, 115.0, 114.9, 108.6, 77.7, 73.7, 52.0, 51.9, 45.8, 40.3, 38.1, 32.2, 31.9, 22.9, 21.4. Mass spec.: 436.52 (MH)⁺.

### Example 52

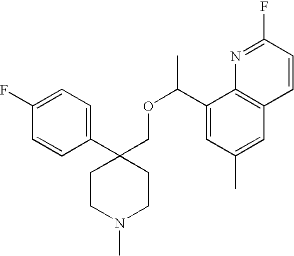

Isolated by preparative HPLC as the trifluoroacetic acid salt: ¹H-NMR (CD₃OD, 500 MHz) δ 8.30 (m, 1H), 7.58 (s, 1H), 7.47 (dd, J=8.6 Hz, 5.2 Hz, 1.5H), 7.42 (dd, J=8.9 Hz, 5.5 Hz, 0.5H), 7.17 (m, 2H), 7.12 (m, 1.5H), 7.01 (s, 0.25H), 5.48 (q, J=6.4 Hz, 0.25H), 5.40 (q, J=6.4 Hz, 0.75H), 3.88 (d, J=9.5 Hz, 0.25H), 3.22-3.60 (m, 6H), 2.70-2.98 (m, 6H), 2.58 (m, 0.75H), 2.41 (m, 2.5H), 2.34 (s, 0.75H), 1.95-2.27 (m, 2H), 1.40 (d, J=6.4 Hz, 3H). Mass spec.: 411.45 (MH)⁺.

### Example 53

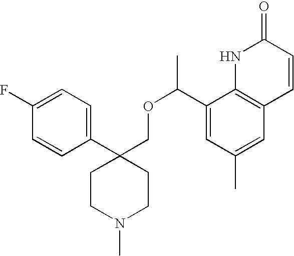

Isolated by preparative HPLC as the trifluoroacetic acid salt: ¹H-NMR (CD₃OD, 500 MHz) δ 7.95 (d, J=9.5 Hz, 0.8H), 7.91 (d, J=9.8 Hz, 0.2H), 7.40-7.50 (m, 2.7H), 7.35 (dd, J=9.2 Hz, 5.2 Hz, 0.5H), 7.17 (d, J=1.5 Hz, 0.8H), 7.12 (m, 1.8H), 7.00 (m, 0.44H), 6.62 (d, J=9.5 Hz, 0.8H), 6.57 (d, J=9.5 Hz, 0.2H), 4.71 (m, 1H), 3.66 (d, J=10.1 Hz, 0.2H), 3.35-3.55 (m, 3.6H), 2.75-2.96 (m, 5H), 2.65 (m, 2H), 2.37 (s, 3H), 2.15 (m, 2H), 1.49 (d, J=6.7 Hz, 2.4H), 1.46 (d, J=6.7 Hz, 0.7H). Mass spec.: 409.47 (MH)⁺.

### Intermediate 38

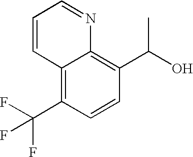

1-(5-(trifluoromethyl)quinolin-8-yl)ethanol. Intermediate 38 was prepared from 2-bromo-5-(trifluoromethyl)aniline following the experimental conditions described in the synthesis of intermediate 16. ¹H NMR (400 MHz, CDCl₃) δ ppm 1.86-2.00 (m, 2H) 2.23 (s, 2H) 2.57-2.74 (m, 2H) 2.81-2.95 (m, 2H) 3.51 (s, 2H) 4.53 (s, 2H) 7.19 (t, J=7.18 Hz, 1H) 7.31 (t, J=7.81 Hz, 2H) 7.35-7.41 (m, 2H) 7.47 (s, 1H) 7.56-7.66 (m, 1H) 7.70-7.77 (m, 1H) 7.90 (d, J=8.31 Hz, 1H) 8.12 (d, J=8.56 Hz, 1H). Mass 242.01 [M+H]⁺.

### Intermediate 39

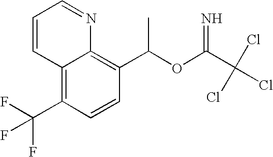

1-(5-(trifluoromethyl)quinolin-8-yl)ethyl 2,2,2-trichloroacetimidate. To a solution of 1-(5-(trifluoromethyl)quinolin-8-yl)ethanol (375 mg, 1.555 mmol) in diethyl ether (8 ml) was added 1,8-Diazabicyclo[5.4.0]undec-7-ene (0.047 ml, 0.311 mmol) followed after 10 minutes by trichloroacetonitrile (0.234 ml, 2.332 mmol). The mixture was allowed to stir at 25° C. for 4 hours then concentrated and purified by column chromatography (SiO2, 40 g) eluting with 10-40% ethyl acetate in hexanes to give the product (333 mg, 56%). ¹HNMR (400 MHz, CDCl₃) δ ppm 9.02 (dd, J=4.15, 1.64 Hz, 1H), 8.47-8.55 (m, 1H), 7.94 (s, 2H), 7.56 (dd, J=8.69, 4.15 Hz, 1H), 7.20 (q, J=6.55 Hz, 1H), 1.72-1.82 (d, J=6.55 Hz, 3H). Mass 385.98 [M+H]⁺.

### Intermediate 40

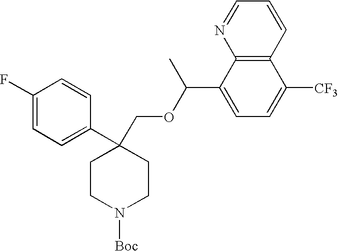

tert-butyl 4-(4-fluorophenyl)-4-((1-(5-(trifluoromethyl)quinolin-8-yl)ethoxy)methyl)piperidine-1-carboxylate. To a solution of 1-(5-(trifluoromethyl)quinolin-8-yl)ethyl 2,2,2-trichloroacetimidate (333 mg, 0.864 mmol) and tert-butyl 4-(4-fluorophenyl)-4-(hydroxymethyl)piperidine-1-carboxylate (294 mg, 0.950 mmol) in dichloromethane (2 ml) at 0° C. was added fluoroboric acid diethyl ether complex (0.013 ml, 0.173 mmol). The reaction was stirred at 0° C. for one hour then quenched with saturated NaHCO₃ solution and extracted into ethyl acetate. The organics were dried over MgSO₄, filtered and concentrated. Purification by column chromatography (SiO₂, 8 g) eluting with 10-40% ethyl acetate in hexanes gave the product (43 mg, 9%). ¹H NMR (400 MHz, CDCl₃) δ ppm 8.91 (dd, J=4.15 Hz, 1H), 8.43-8.49 (m, 1H), 7.76 (d, J=7.81 Hz, 1H), 7.46-7.53 (m, 1H), 7.27-7.38 (m, 3H), 6.98-7.07 (m, 2H), 5.50-5.61 (m, 1H), 3.73 (s, 2H), 3.35-3.41 (m 1H), 3.28 (d, J=9.06 Hz), 2.95-3.14 (m, 2H), 2.20 (s, 2H), 1.83-2.01 (m, 2H), 1.43 (s, 9H), 1.40 (d, J=6.55 Hz, 3H). Mass 533.31 [M+H]⁺.

### Example 54

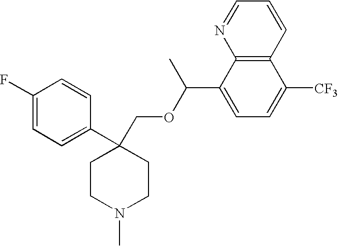

8-(1-((4-(4-fluorophenyl)-1-methylpiperidin-4-yl)methoxy)ethyl)-5-(trifluoromethyl)quinoline. Example 54 was prepared from tert-butyl 4-(4-fluorophenyl)-4-((1-(5-(trifluoromethyl)quinolin-8-yl)ethoxy)methyl)piperidine-1-carboxylate (43 mg, 0.081 mmol) following the experimental procedure described for examples 1 and 2. (22 mg, 0.046 mmol, 56%-two steps) ¹H NMR (400 MHz, MeOD) δ ppm 7.81 (1H, d), 7.62 (1H, dd, J=8.81, 4.28 Hz), 7.37-7.47 (3H, m), 7.08 (3H, t, J=8.81 Hz), 5.60 (1H, q, J=6.30 Hz), 3.42-3.51 (1H, m), 3.33 (3H, s), 2.93-3.08 (1H, m), 2.39-2.73 (6H, m), 2.27-2.38 (1H, m), 2.05-2.25 (2H, m), 1.39 (3H, d, J=6.30 Hz). Mass 447.18 [M+H]⁺.

The examples in Table 11 were prepared following the experimental procedure described for intermediates 13, 14, 16, 5, 40 and example 54 using the appropriately substituted anilines.

TABLE 11

| | | MS | HPLC | |
|---|---|---|---|---|
| Example | Structure | (MH)⁺ | (method) | ¹H NMR |
| 54 | 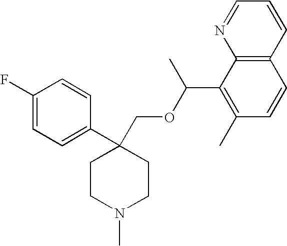 | 393.23 | 0.837(1) | ¹H NMR (400 MHz,MeOD) δ ppm 8.76 (1 H,dd, J = 4.28, 1.76 Hz), 8.18(1 H, dd, J = 8.18, 1.89Hz), 7.67 (1 H, d, J = 8.31Hz), 7.32-7.40 (3 H, m),7.29 (1 H, d, J = 8.56 Hz),7.03 (2 H, t, J = 8.81 Hz),6.17 (1 H, q, J = 6.55 Hz),3.33 (3 H, s), 3.16 (1 H, d,J = 9.32 Hz),2.93-3.09 (2H, m), 2.52-2.71 (2 H,m), 2.49 (3 H, s), 2.24-2.36 (2 H, m), 2.01-2.20(2 H, m), 1.46 (3 H, d,J = 6.80 Hz) |
| 55 | 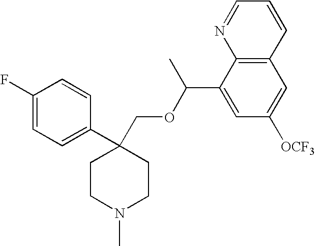 | 463.21 | 2.52(2) | ¹H NMR (400 MHz,MeOD) δ ppm 8.84 (1 H,dd, J = 4.28, 1.76 Hz), 8.30(1 H, dd, J = 8.31, 1.76Hz), 7.65 (1 H, br. s.),7.52 (1 H, dd, J = 8.44,4.15 Hz), 7.36-7.42 (2 H,m), 7.07-7.10 (1 H, m),7.04 (2 H, t, J = 8.81 Hz),5.50 (1 H, q, J = 6.55 Hz),3.43-3.49 (1 H, m), 2.55-2.73 (2 H, m), 2.17-2.23 (3 H, m), 2.20 (6 H,s), 1.37 (3 H, d, J = 6.55Hz) |
| 56 | 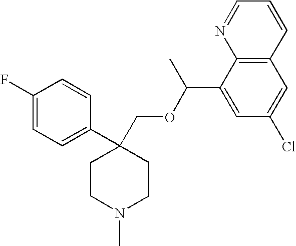 | 413.45 | 1.598(1) | ¹H NMR (400 MHz,MeOD) δ ppm, 8.80 (1 H,dd, J = 4.15, 1.64 Hz), 8.20(1 H, dd, J = 8.31, 1.76Hz), 7.76 (1 H, d, J = 2.52Hz), 7.47 (1 H, dd,J = 8.31, 4.28 Hz), 7.36-7.42 (2 H, m), 7.12 (1 H,d, J = 2.27 Hz), 7.06 (2 H,t, J = 8.81 Hz), 5.44-5.52(1 H, m), 3.42-3.48 (1 H,m), 2.52-2.69 (2 H, m),2.13-2.24 (3 H, m), 2.19(6 H, s), 1.35 (3 H, d,J = 6.55 Hz) |
| 57 | 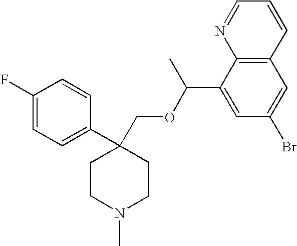 | 457.40 | 1.618(1) | ¹H NMR (400 MHz,MeOD) δ ppm 8.81 (dd,J = 4.3, 1.8 Hz, 1 H), 8.19(dd, J = 8.4, 1.6 Hz, 1 H),7.94 (d, J = 2.3 Hz, 1 H),7.47 (dd, J = 8.3, 4.3 Hz, 1H), 7.36-7.43 (m, 2 H),7.29 (d, J = 2.0 Hz, 1 H),7.06 (t, J = 8.9 Hz, 2 H),5.48 (q, J = 6.8 Hz, 1 H),3.33 (s, 2 H), 2.53-2.71(m, 2 H), 2.19 (s, 3 H),2.02-2.39 (m, 5 H), 1.92-2.03 (m, 1 H), 1.35 (d,J = 6.3 Hz, 3 H) |

### Intermediate 41

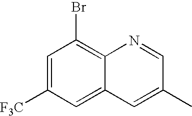

8-bromo-3-methyl-6-(trifluoromethyl)quinoline. 2-bromo-4-(trifluoromethyl)aniline (5 g, 20.83 mmol) was added to 6N HCl (20.83 mL). The mixture was heated to reflux and added methacrolein (3.96 mL, 47.9 mmol) dropwise over 20 minutes via addition funnel. Reflux was continued at 100° C. overnight then the mixture was cooled and adjusted to pH ˜5-6 using NH₄OH (aq). The mixture was extracted with ether (2×100 ml). The combined organic layers were washed with brine, dried over MgSO₄, filtered then concentrated and purified by column chromatography (SiO₂) eluting with 10-30% EtOAc in hexanes to give the product (930 mg, 15%). ¹H NMR (400 MHz, MeOD) δ ppm 8.81 (1H, dd, J=4.28, 1.76 Hz), 8.19 (1H, dd, J=8.44, 1.64 Hz), 7.94 (1H, d, J=2.27 Hz), 7.47 (1H, dd, J=8.31, 4.28 Hz), 7.36-7.43 (2H, m), 7.29 (1H, d, J=2.01 Hz), 7.06 (2H, t, J=8.94 Hz), 5.44-5.52 (1H, m, J=6.80 Hz), 3.33 (2H, s), 2.53-2.71 (2H, m), 2.19 (3H, s), 2.02-2.39 (5H, m), 1.92-2.03 (1H, m), 1.35 (3H, d, J=6.30 Hz). Mass 291.79 [M+H]⁺.

### Example 57

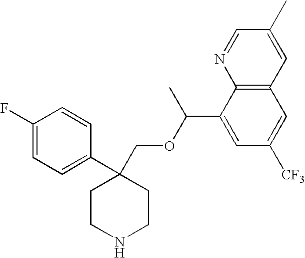

8-(1-((4-(4-fluorophenyl)piperidin-4-yl)methoxy)ethyl)-3-methyl-6-(trifluoromethyl)quinoline. Example 57 was prepared from intermediate 41 by following experimental conditions described for intermediates 14, 16 and example 1. ¹H NMR (400 MHz, MeOD) δ ppm 8.81 (1H, d, J=2.01 Hz), 8.17 (1H, s), 8.06 (1H, s), 7.49 (1H, d, J=1.51 Hz), 7.36-7.43 (2H, m), 7.02 (2H, t, J=8.81 Hz), 5.51 (1H, q, J=6.38 Hz), 3.42 (1H, d, J=9.07 Hz), 3.30-3.35 (1H, m), 2.79-2.91 (2H, m), 2.59-2.72 (2H, m), 2.52 (3H, s), 2.19-2.28 (1H, m), 2.09-2.17 (1H, m), 1.84-2.01 (2H, m), 1.37 (3H, d, J=6.30 Hz). Mass 447.21 [M+H]⁺.

### Example 58

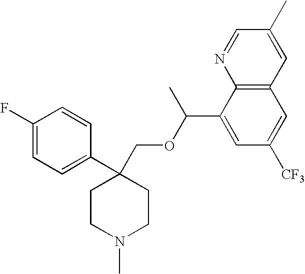

8-(1-((4-(4-fluorophenyl)-1-methylpiperidin-4-yl)methoxy)ethyl)-3-methyl-6-(trifluoromethyl)quinoline. Example 58 was prepared from example 57 by following experimental conditions described for example 2. ¹H NMR (400 MHz, MeOD) δ ppm 8.82 (1H, d, J=2.01 Hz), 8.17 (1H, br. s.), 8.07 (1H, br. s.), 7.48 (1H, br. s.), 7.37-7.44 (2H, m), 7.05 (2H, t, J=8.69 Hz), 5.54 (1H, q, J=6.29 Hz), 3.40-3.48 (1H, m), 3.31-3.36 (1H, m), 2.79-2.91 (2H, m), 2.52 (3H, s), 2.39-2.57 (3H, m), 2.37 (3H, s), 2.23-2.32 (1H, m), 2.00-2.20 (2H, m), 1.39 (3H, d, J=6.30 Hz). Mass 461.13 [M+H]⁺.

### Intermediate 42

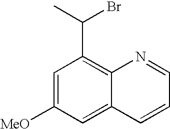

8-(1-bromoethyl)-6-methoxyquinoline. Intermediate 42 may be prepared according to the literature (Zhurnal Obshchei Khimii (1941), 11 537-40) and the experimental procedures described for intermediates 14, 16 and 1. ¹H NMR (400 MHz, CHLOROFORM-d) δ ppm 8.82 (1H, dd, J=4.15, 1.64 Hz), 8.05 (1H, d, J=8.06 Hz), 7.66 (1H, d, J=2.52 Hz), 7.38 (1H, dd, J=8.18, 4.15 Hz), 7.02 (1H, d, J=2.77 Hz), 6.68 (1H, q, J=6.88 Hz), 3.93 (3H, s), 2.15 (3H, d, J=7.05 Hz). Mass 267.99 [M+H]⁺.

### Example 59

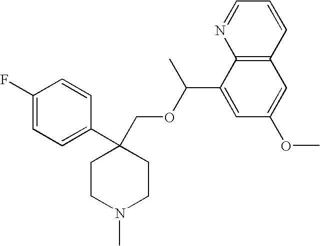

8-(1-((4-(4-fluorophenyl)-1-methylpiperidin-4-yl)methoxy)ethyl)-6-methoxyquinoline. Example 59 was prepared from intermediate 42 according to the experimental conditions described for examples 1 and 2. ¹H NMR (400 MHz, MeOD) δ ppm 8.61 (1H, dd, J=4.28, 1.76 Hz), 8.16 (1H, dd, J=8.31, 1.51 Hz), 7.35-7.42 (3H, m), 7.09 (1H, d, J=3.02 Hz), 7.05 (2H, t, J=8.81 Hz), 6.97 (2H, d, J=2.77 Hz), 5.45 (1H, q), 3.86 (3H, s), 3.37-3.44 (1H, m), 3.29-3.35 (1H, m), 2.78-2.92 (2H, m), 2.36 (2H, s), 2.33-2.55 (3H, m), 2.21-2.31 (1H, m), 2.01-2.20 (2H, m), 1.35 (3H, d, J=6.55 Hz). Mass 409.44 [M+H]⁺.

### Intermediate 43

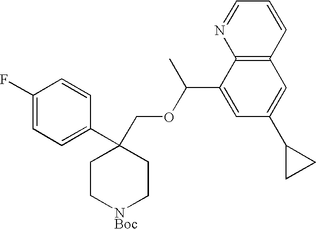

tert-butyl 4-((1-(6-cyclopropylquinolin-8-yl)ethoxy)methyl)-4-(4-fluorophenyl)piperidine-1-carboxylate. tert-butyl 4-((1-(6-bromoquinolin-8-yl)ethoxy)methyl)-4-(4-fluorophenyl)piperidine-1-carboxylate (prepared according to the experimental conditions described for example 56, (143 mg, 0.263 mmol) in toluene (3 ml) was added cyclopropylboronic acid (67.8 mg, 0.789 mmol), 1,1′-bis(diphenylphosphino)ferrocene-palladium(II)dichloride dichloromethane complex (21.49 mg, 0.026 mmol) and cesium carbonate (266 mg, 0.816 mmol). The mixture was flushed with nitrogen and heated at 100° C. for 2 hours. An additional 0.1 equivalents of 1,1′-bis(diphenylphosphino)ferrocene-palladium(II) dichloride dichloromethane complex (21.49 mg, 0.026 mmol) was added and heating at 110° C. was resumed overnight.

The mixture was cooled and quenched with saturated aqueous NaHCO₃ and diluted with EtOAc. The combined organics were washed with brine, dried over MgSO₄, filtered and concentrated. Purification by column chromatography (SiO₂) eluting with 15-30% EtOAc in hexanes gave the product (38 mg, 0.075 mmol, 29%). Mass 505.57 [M+H]⁺.

### Example 60

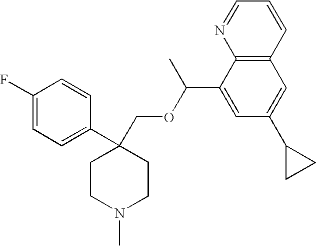

6-cyclopropyl-8-(1-((4-(4-fluorophenyl)-1-methylpiperidin-4-yl)methoxy)ethyl)quinoline. Example 60 was prepared from intermediate 43 according to the experimental conditions described for example 55. ¹H NMR (400 MHz, MeOD) δ ppm 8.65-8.72 (1H, m), 8.13 (1H, dd, J=8.31, 1.51 Hz), 7.33-7.44 (4H, m), 7.09 (1H, d, J=1.51 Hz), 7.05 (2H, t, J=8.81 Hz), 5.47 (1H, q, J=6.46 Hz), 3.23-3.43 (2H, m), 2.52-2.68 (2H, m), 2.18 (3H, s), 2.04-2.35 (5H, m), 1.86-2.03 (2H, m), 1.35 (3H, d, J=6.30 Hz), 0.97-1.07 (2H, m), 0.58-0.76 (2H, m). Mass 419.35 [M+H]⁺.

### Intermediate 44

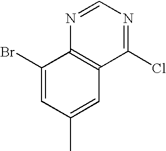

8-bromo-4-chloro-6-methylquinazoline. A mixture of neat 2-amino-3-bromo-5-methylbenzoic acid (2 g, 8.69 mmol) and formamidine acetate (1.005 g, 9.65 mmol) was heated with a heat gun and shaking in a 100 ml round bottom flask under a N₂ balloon until the material was melted together (4 minutes). To the crude 8-bromo-6-methylquinazolin-4(3H)-one (1.68 g) was added POCl₃ (13 mL, 139 mmol) and the mixture was heated at reflux overnight. Excess POCl₃ was removed by rotovap and ice was added to the residue. The pH of the mixture was neutralized to pH 7 using NaOH and saturated aqueous Na₂CO₃. The aqueous layer was then extracted with EtOAc, washed with brine, dried over MgSO₄ and concentrated. Purification of the crude residue by column chromatography (SiO₂) eluting with 0-20% EtOAc in hexanes gave the product (1.24 g, 4.82 mmol, 55%). ¹H NMR (400 MHz, CHLOROFORM-d) δ ppm 9.09 (1H, s), 8.12 (1H, d, J=1.76 Hz), 8.01 (1H, d, J=1.01 Hz), 2.59 (3H, s). Mass 258.97 [M+H]⁺.

### Intermediate 45

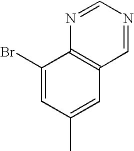

8-bromo-6-methylquinazoline. To a solution of 8-bromo-4-chloro-6-methylquinazoline (0.934 g, 3.63 mmol) in dichloromethane (35 ml) was added p-toluenesulfonohydrazide (1.824 g, 9.79 mmol). The reaction mixture was stirred at room temperature overnight the solvent was removed under vacuum to give crude product. The crude product from was added to a solution of sodium carbonate (2.307 g, 21.76 mmol) in 40 ml of water. The mixture was heated at 180° C. for 3 hours in a sealed tube then cooled to ambient temperature and extracted with dichloromethane. Dried dichloromethane layer over MgSO₄ and concentrated. Purification by column chromatography (SiO₂) eluting with 20-40% EtOAc in hexanes gave the product (439 mg, 1.97 mmol, 54%). ¹H NMR (400 MHz, CHLOROFORM-d) δ ppm 9.39 (1H, s), 9.30 (1H, s), 8.09 (1H, d, J=1.76 Hz), 7.67 (1H, s), 2.56 (3H, s).

### Intermediate 46

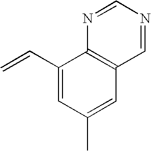

6-methyl-8-vinylquinazoline. 8-bromo-6-methylquinazoline (310 mg, 1.390 mmol) was dissolved in 2-propanol (15 ml) (degassed by bubbling through N₂). To this mixture was added triethylamine (0.678 ml, 4.86 mmol), Potassium vinyltrifluoroborate (186 mg, 1.390 mmol) and 1,1′-Bis(diphenylphosphino)ferrocene-palladium(II) dichloride dichloromethane complex (57.2 mg, 0.069 mmol). The mixture was heated in a microwave reactor at 130° C. for 30 minutes the cooled and filtered through a pad of celite and concentrated. Purification by column chromatography (SiO₂) eluting with 20-40% EtOAc in hexanes gave the product (170 mg, 0.999 mmol, 72%). ¹H NMR (400 MHz, CHLOROFORM-d) δ ppm 9.27 (2H, s), 7.92 (1H, d, J=2.01 Hz), 7.81 (1H, dd, J=17.88, 11.08 Hz), 7.59 (1H, br. s.), 6.00 (1H, dd, J=17.88, 1.26 Hz), 5.54 (1H, dd, J=11.08, 1.26 Hz), 2.57 (3H, s). Mass 171.06 [M+H]⁺.

### Intermediate 47

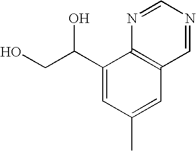

1-(6-methylquinazolin-8-yl)ethane-1,2-diol. To a solution of 6-methyl-8-vinylquinazoline (170 mg, 0.999 mmol) in acetone (9 ml) and water (1 ml) was added osmium tetroxide (2.5 wt % in 2-methyl-2-propanol) (0.626 ml, 0.050 mmol). The reaction mixture was cooled to 0° C. and NMO (234 mg, 1.998 mmol) was added. The reaction mixture was stirred at 0° C. and allowed to slowly warm to 25° C. over 1.5 hours. The reaction was quenched with aqueous sodium sulfite and extracted with EtOAc. The combined organics were washed with brine, dried over MgSO₄, filtered and concentrated to give the crude product (163 mg, 0.798 mmol, 80%). ¹H NMR (400 MHz, CHLOROFORM-d) δ ppm 9.34 (1H, s), 9.21 (1H, s), 7.72 (1H, d, J=1.76 Hz), 7.64 (1H, s), 5.23-5.36 (2H, m), 3.90-3.98 (2H, m), 2.62-2.70 (1H, m), 2.57 (3H, s).

### Intermediate 48

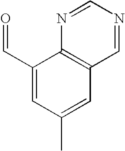

6-methylquinazoline-8-carbaldehyde. To a solution of 1-(6-methylquinazolin-8-yl)ethane-1,2-diol (163 mg, 0.798 mmol) in 1,4-dioxane (4 ml) and water (4 ml) was added sodium periodate (512 mg, 2.394 mmol). The reaction mixture was stirred at 25° C. for 1 hour the diluted with EtOAc, washed with water, brine, dried over MgSO₄, filtered and concentrated. Purification by column chromatography (SiO₂) eluting with 40-60% EtOAc in hexanes gave the product (121 mg, 0.703 mmol, 88%). ¹H NMR (400 MHz, CHLOROFORM-d) δ ppm 11.29 (1H, s), 9.42 (1H, s), 9.39 (1H, s), 8.36 (1H, d, J=2.01 Hz), 7.96 (1H, d, J=1.01 Hz), 2.64 (3H, s). Mass 173.27 [M+H]⁺.

### Example 60

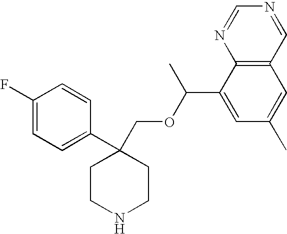

8-(1-((4-(4-fluorophenyl)piperidin-4-yl)methoxy)ethyl)-6-methylquinazoline. Example 60 was prepared from intermediate 48 according to the experimental conditions described for intermediates 16, 1 and example 1. ¹H NMR (400 MHz, CHLOROFORM-d) δ ppm 9.23 (1H, s), 9.18 (1H, s), 7.50 (1H, s), 7.29 (2H, dd, J=8.81, 5.29 Hz), 7.03 (2H, t, J=8.69 Hz), 5.41 (1H, q, J=6.30 Hz), 3.40 (1H, d, J=9.06 Hz), 3.22 (1H, d, J=8.81 Hz), 2.93-3.08 (2H, m), 2.68-2.92 (4H, m), 2.40 (3H, s), 2.24-2.33 (1H, m), 2.02-2.13 (2H, m), 1.88-2.00 (1H, m), 1.38 (3H, d, J=6.30 Hz). Mass 380.28 [M+H]⁺.

### Intermediate 49

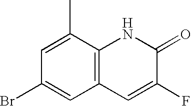

6-bromo-3-fluoro-8-methylquinolin-2(1H)-one. A solution of 4-bromo-2-methylaniline (6.12 g, 32.9 mmol) in THF (4 mL) and hexanes (2 mL) was cooled in an ice bath. To this mixture was added LiHMDS (1.0 M solution in THF) (39.5 mL, 39.5 mmol) and the reaction was stirred at 0° C. for 1.5 hours. (Z)-ethyl 2-fluoro-3-methoxyacrylate (prepared according to the literature—Tetrahedron 1994, 50, 1129) (4.87 g, 32.9 mmol) was added neat to the mixture and stirring was continued at 0° C. for another 2.5 hours. The mixture was poured into cold 1N HCl and extracted with diethyl ether. The combined extracts were dried over MgSO₄, filtered and concentrated to give 9.72 g of red-orange/brown crude intermediate. This material was dissolved in 70 ml of 75% sulfuric acid and heated at 60° C. for 2 hours then poured onto crushed ice and filtered as a brown pasty cake. The filter cake was washed with water and cold diethyl ether then dried under vacuum to obtain 10.67 g of crude product. ¹H NMR (400 MHz, DMSO-d₆) δ ppm 7.84 (1H, d, J=10.83 Hz), 7.75 (1H, d, J=2.27 Hz), 7.51-7.54 (1H, m), 2.43 (3H, s). Mass 256.08 [M+H]⁺.

### Intermediate 50

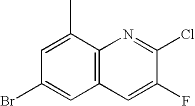

6-bromo-2-chloro-3-fluoro-8-methylquinoline. A solution of 6-bromo-3-fluoro-8-methylquinolin-2(1H)-one (10.67 g, 41.7 mmol) in POCl₃ (50 mL, 536 mmol) was heated at reflux overnight. Excess POCl₃ was evaporated and crushed ice was added to the residue and the pH was adjusted to ˜7 with NaOH and sat. Na₂CO₃. The aqueous portion was extracted with EtOAc, washed with brine, dried over MgSO₄, filtered and concentrated. Purification by column chromatography (SiO₂) eluting with 1% EtOAc in hexanes gave 1.86 g of a mixture of product and unknown by-product along with 810 mg of more pure desired product. The more pure product was carried on as is. ¹H NMR (400 MHz, CHLOROFORM-d) δ ppm 11.30-11.32 (1H, m), 8.94 (1H, d, J=2.77 Hz), 8.32 (1H, d, J=2.27 Hz), 8.20 (1H, d, J=2.27 Hz), 7.77 (1H, dd, J=8.44, 2.90 Hz)

Mass 276.09 [M+H]⁺.

### Intermediate 51

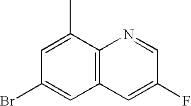

6-bromo-3-fluoro-8-methylquinoline. To a solution of 6-bromo-2-chloro-3-fluoro-8-methylquinoline (810 mg, 2.95 mmol) in HI (57% soln in water) (25 ml, 189 mmol) was added phosphorus (red) (1371 mg, 44.3 mmol). The reaction mixture was heated at reflux for 2 hours. The mixture was filtered through a pad of celite and neutralized to pH ˜7 using 10N NaOH and sat NaHCO₃. The crude solution was extracted with dichloromethane, washed with brine, dried over MgSO₄, filtered and concentrated. Purification by column chromatography (SiO₂) eluting with 0-5% EtOAc in hexanes gave 1.28 g of a mixture of product and unknown by-product that was carried on directly to the next step Mass 240.08 [M+H]⁺.

### Intermediate 52

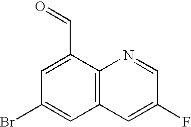

6-bromo-3-fluoroquinoline-8-carbaldehyde. To a solution of 6-bromo-3-fluoro-8-methylquinoline (1.17 g, 4.87 mmol) in 1,2-dichlorobenzene (40 mL) was added selenium dioxide (1.622 g, 14.62 mmol). This mixture was heated in a microwave reactor at 200° C. for 4 hours (split mixture into two 20 ml microwave vials).

The mixture was filtered through a pad of celite and concentrated. Purification by column chromatography (SiO₂) eluting with 3% EtOAc in hexanes gave the product as a somewhat impure mixture (595 mg) which was carried on directly to the next step. ¹H NMR (400 MHz, CHLOROFORM-d) δ ppm 11.30-11.32 (1H, m), 8.94 (1H, d, J=2.77 Hz), 8.32 (1H, d, J=2.27 Hz), 8.20 (1H, d, J=2.27 Hz), 7.77 (1H, dd, J=8.44, 2.90 Hz). Mass 256.01 [M+H]⁺ (⁸¹Br).

### Example 62

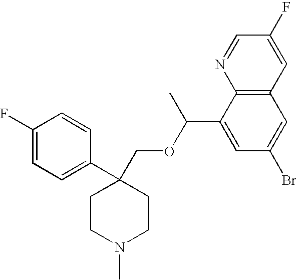

6-bromo-3-fluoro-8-(1-((4-(4-fluorophenyl)-1-methylpiperidin-4-yl)methoxy)ethyl)quinoline. Example 62 was prepared from 6-bromo-3-fluoro-8-methylquinoline according to the experimental procedures for intermediates 16 and 1 and examples 1 and 2. ¹H NMR (400 MHz, MeOD) δ ppm 8.76 (1H, d, J=3.02 Hz), 7.95 (1H, d, J=2.27 Hz), 7.91 (1H, dd), 7.36-7.43 (2H, m), 7.25 (1H, d, J=2.01 Hz), 7.05 (2H, t, J=8.81 Hz), 5.42-5.50 (1H, m), 3.40-3.48 (1H, m), 3.30-3.36 (1H, m), 2.53-2.70 (2H, m), 2.15-2.23 (3H, m), 1.90-2.41 (6H, m), 1.34 (3H, d, J=6.55 Hz). Mass 475.04 [M+H]⁺.

### Intermediate 53

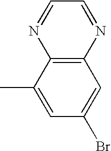

7-bromo-5-methylquinoxaline. To a solution of 5-bromo-3-methylbenzene-1,2-diamine (Bioorg. Med. Chem. 2000, 2591.) (3.63 g, 18.05 mmol) in ethanol (50 ml) was added glyoxal 40 wt. % in water (8.0 ml, 69.7 mmol). The mixture was heated to reflux for 1.5 hours and an aliquot was removed, concentrated and analyzed by LC/MS. Many overlapping peaks were observed, including a major peak corresponding to desired mass 225 ([M+H]⁺ for ⁸¹Br isotope). The mixture was removed from heat and allowed to stir overnight. Reflux was resumed for 1.5 hours and another aliquot was taken. LC/MS showed a higher ratio of desired to other peaks by integration. Analysis after 6 hours showed no appreciable change. The mixture was concentrated to remove most of the ethanol then diluted with water and dichloromethane. The layers were shaken and separated and the aqueous portion was extracted 2× with dichloromethane. Combined organics were washed with brine, dried over MgSO₄, filtered and concentrated to give 4.96 g of a black thick residue. The crude material was purified via column chromatography (SiO₂) eluting with 10% ethyl acetate/hexane to give the product as a white solid (1.70 g, 7.62 mmol, 42%). ¹H NMR (400 MHz, MeOD) δ ppm 8.80-8.84 (2H, m), 8.07 (1H, d, J=2.27 Hz), 7.39 (2H, dd, J=9.07, 5.29 Hz), 7.34 (1H, s), 7.07 (2H, t, J=8.81 Hz), 5.47 (1H, q, J=6.30 Hz), 3.47 (1H, d, J=8.81 Hz), 3.29-3.31 (1H, m), 2.75-2.92 (2H, m), 2.34 (3H, s), 2.31-2.55 (3H, m), 2.07-2.29 (2H, m), 1.96-2.06 (1H, m), 1.36 (3H, d, J=6.30 Hz). Mass 225 [M+H]⁺ (⁸¹Br).

### Intermediate 54

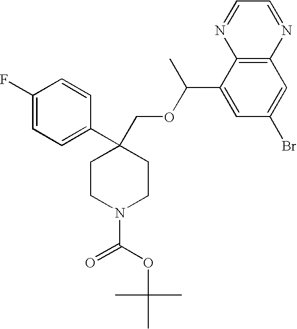

tert-butyl 4-((1-(7-bromoquinoxalin-5-yl)ethoxy)methyl)-4-(4-fluorophenyl)piperidine-1-carboxylate. Intermediate 54 was prepared from 7-bromo-5-methylquinoxaline according to the experimental procedure described for intermediates 5, 16, and 40. ¹H NMR (400 MHz, CHLOROFORM-d) δ ppm 8.79 (1H, d, J=1.76 Hz), 8.75 (1H, d, J=1.76 Hz), 8.11 (1H, d, J=2.27 Hz), 7.36 (1H, d, J=2.01 Hz), 7.27-7.33 (2H, m), 7.04 (2H, t, J=8.81 Hz), 5.41 (1H, q, J=6.46 Hz), 3.65-3.82 (2H, m), 3.37 (1H, d, J=9.06 Hz), 3.26 (1H, d, J=8.81 Hz), 2.95-3.12 (2H, m), 2.19-2.30 (1H, m), 2.01-2.11 (1H, m), 1.94 (1H, ddd, J=14.10, 10.45, 4.15 Hz), 1.82 (1H, ddd, J=14.04, 10.26, 3.90 Hz), 1.43 (9H, s), 1.35-1.40 (3H, m). Mass 446.24 [M−Boc+H]⁺

### Example 63

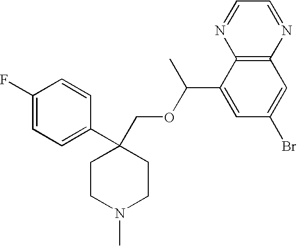

7-bromo-5-(1-((4-(4-fluorophenyl)-1-methylpiperidin-4-yl)methoxy)ethyl)quinoxaline. Example 63 was prepared from tert-butyl 4-((1-(7-bromoquinoxalin-5-yl)ethoxy)methyl)-4-(4-fluorophenyl)piperidine-1-carboxylate according to the experimental procedure described for examples 1 and 2. ¹H NMR (400 MHz, MeOD) δ ppm 8.76 (1H, d, J=3.02 Hz), 7.95 (1H, d, J=2.27 Hz), 7.91 (1H, dd), 7.36-7.43 (2H, m), 7.25 (1H, d, J=2.01 Hz), 7.05 (2H, t, J=8.81 Hz), 5.42-5.50 (1H, m), 3.40-3.48 (1H, m), 3.30-3.36 (1H, m), 2.53-2.70 (2H, m), 2.15-2.23 (3H, m), 1.90-2.41 (6H, m), 1.34 (3H, d, J=6.55 Hz). Mass 458.29 [M+H]⁺.

### Intermediate 55

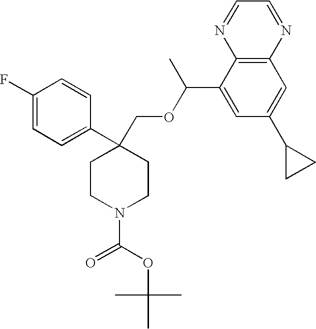

tert-butyl 4-((1-(7-cyclopropylquinoxalin-5-yl)ethoxy)methyl)-4-(4-fluorophenyl)piperidine-1-carboxylate. Intermediate 55 was prepared from tert-butyl 4-((1-(7-bromoquinoxalin-5-yl)ethoxy)methyl)-4-(4-fluorophenyl)piperidine-1-carboxylate according to the experimental procedure for intermediate 43 (24 mg, 0.047 mmol, 37%). ¹H NMR (400 MHz, CHLOROFORM-d) δ ppm 8.73 (1H, d, J=1.76 Hz), 8.65 (1H, d, J=1.76 Hz), 7.53 (1H, d, J=2.01 Hz), 7.31 (2H, dd, J=8.81, 5.29 Hz), 7.14 (1H, d, J=1.76 Hz), 7.02 (2H, t, J=8.69 Hz), 5.43 (1H, q, J=6.38 Hz), 3.63-3.80 (2H, m), 3.34-3.38 (1H, m), 3.24-3.30 (1H, m), 2.98-3.13 (2H, m), 2.15-2.25 (1H, m), 2.02-2.10 (1H, m), 1.89-2.00 (2H, m), 1.84 (1H, ddd, J=13.79, 10.14, 4.03 Hz), 1.43 (9H, s), 1.39 (3H, d, J=6.55 Hz), 1.06-1.16 (2H, m), 0.76-0.83 (1H, m), 0.68-0.75 (1H, m). Mass 506.42 [M+H]⁺.

### Example 64

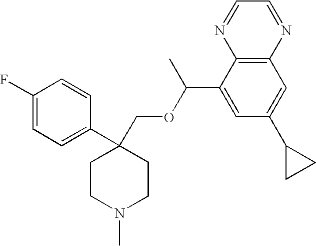

7-cyclopropyl-5-(1-((4-(4-fluorophenyl)-1-methylpiperidin-4-yl)methoxy)ethyl)quinoxaline. Example 64 was prepared from tert-butyl 4-((1-(7-cyclopropylquinoxalin-5-yl)ethoxy)methyl)-4-(4-fluorophenyl)piperidine-1-carboxylate according to the experimental procedures for examples 1 and 2 (13 mg, 0.029 mmol, 72%). ¹H NMR (400 MHz, MeOD) δ ppm 8.72-8.74 (1H, m), 8.70-8.72 (1H, m), 7.51 (1H, d, J=2.01 Hz), 7.41 (2H, dd, J=8.94, 5.41 Hz), 7.14 (1H, d, J=2.01 Hz), 7.07 (2H, t, J=8.81 Hz), 5.48 (1H, q, J=6.46 Hz), 3.42 (1H, d, J=8.81 Hz), 3.25-3.31 (2H, m), 2.75-2.92 (2H, m), 2.35 (3H, s), 2.31-2.53 (2H, m), 2.08-2.29 (2H, m), 1.90-2.07 (2H, m), 1.37 (3H, d, J=6.55 Hz), 1.04-1.16 (2H, m), 0.65-0.80 (2H, m). Mass 420.33 [M+H]⁺.

Table 12 describes compounds that were prepared from tert-butyl 4-(4-bromophenyl)-4-(hydroxymethyl)piperidine-1-carboxylate and tert-butyl 4-(hydroxymethyl)-4-(3-methoxyphenyl)piperidine-1-carboxylate (WO 2007/121389 A2) according to the experimental conditions described in example 1 and example 2. Retention time (t_{R}) is in min.

TABLE 12

| | | MS | HPLC | |
|---|---|---|---|---|
| Example | Structure | (MH)⁺ | (method) | ¹H NMR |
| 65 | 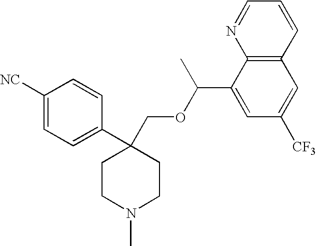 | 454.27 | 2.385(2) | ¹H NMR (400 MHz,MeOD) δ ppm 8.95 (1 H,dd, J = 4.03, 1.76 Hz), 8.41(1 H, dd, J = 8.31, 1.76Hz), 8.16 (1 H, s), 7.65-7.70 (2 H, m), 7.55-7.63(3 H, m), 7.44 (1 H, d,J = 1.76 Hz), 5.56 (1 H, q,J = 6.55 Hz), 3.53 (1 H, d,J = 9.06 Hz), 3.39 (1 H, d,J = 9.07 Hz), 2.64-2.82 (2H, m), 2.27 (3 H, s), 2.20-2.47 (4 H, m), 2.08-2.18 (1 H, m), 1.97-2.07(1 H, m), 1.38 (3 H, d,J = 6.30 Hz) |
| 66 | 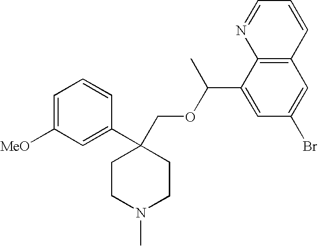 | 469.30 | 2.898(3) | ¹H NMR (400 MHz,MeOD) δ ppm 8.80 (1 H,dd, J = 4.03, 1.76 Hz), 8.16(1 H, dd, J = 8.44, 1.64Hz), 7.92 (1 H, d, J = 2.27Hz), 7.45 (1 H, dd,J = 8.31, 4.28 Hz), 7.40 (1H, d, J = 2.27 Hz), 7.26 (1H, t, J = 8.06 Hz), 6.95 (1H, d, J = 7.81 Hz), 6.91 (1H, t, J = 2.14 Hz), 6.80 (1H, dd, J = 7.93, 2.14 Hz),5.48 (1 H, q, J = 6.46 Hz),3.76 (3 H, s), 3.42 (1 H, d,J = 8.81 Hz), 3.30-3.36 (1H, m), 2.71-2.87 (2 H,m), 2.29-2.50 (3 H, m),2.30 (3 H, s), 2.18-2.27(1 H, m), 1.97-2.16 (2 H,m), 1.36 (3 H, d, J = 6.30Hz) |

We claim:

1. A compound of Formula I 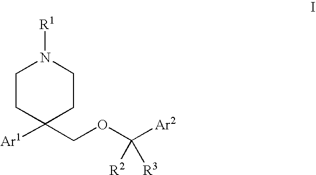
where:
R¹ is hydrogen or alkyl;
R² is hydrogen or alkyl;
R³ is hydrogen or alkyl;
R⁴ is amino, alkylamino, dialkylamino, pyrrolidinyl, piperidinyl, piperazinyl, (alkyl)piperazinyl, morpholinyl, or thiomorpholinyl:
R⁵ is hydrogen or alkyl;
Ar¹ is phenyl or pyridinyl and is substituted with 0-3 substituents selected from the group consisting of halo, alkyl, haloalkyl, alkoxy, and cyano;
Ar² is quinolinyl, isoquinolinyl, quinazolinyl, or quinoxalinyl and is substituted with 0-3 substituents selected from the group consisting of halo, alkyl, cycloalkyl, haloalkyl, hydroxy, alkoxy, haloalkoxy, cyano, R⁴, COR⁴, CO₂R⁵, and Ar³; and
Ar³ is phenyl substituted with 0-3 substituents selected from the group consisting of halo, alkyl, haloalkyl, hydroxy, alkoxy, haloalkoxy, and cyano;
or a pharmaceutically acceptable salt thereof.

2. A compound of claim 1 where R¹ is hydrogen.

3. A compound of claim 1 where R¹ is methyl.

4. A compound of claim 1 where R² and R³ are hydrogen.

5. A compound of claim 1 where R² is methyl and R³ is hydrogen.

6. A compound of claim 1 where Ar¹ is phenyl.

7. A compound of claim 1 where Ar¹ is halophenyl.

8. A compound of claim 1 where Ar² is quinolinyl substituted with 0-3 substituents selected from the group consisting of halo, alkyl, cycloalkyl, haloalkyl, hydroxy, alkoxy, haloalkoxy, cyano, R⁴, COR⁴, CO₂R⁵, and Ar³.

9. A compound of claim 1 selected from the group consisting of 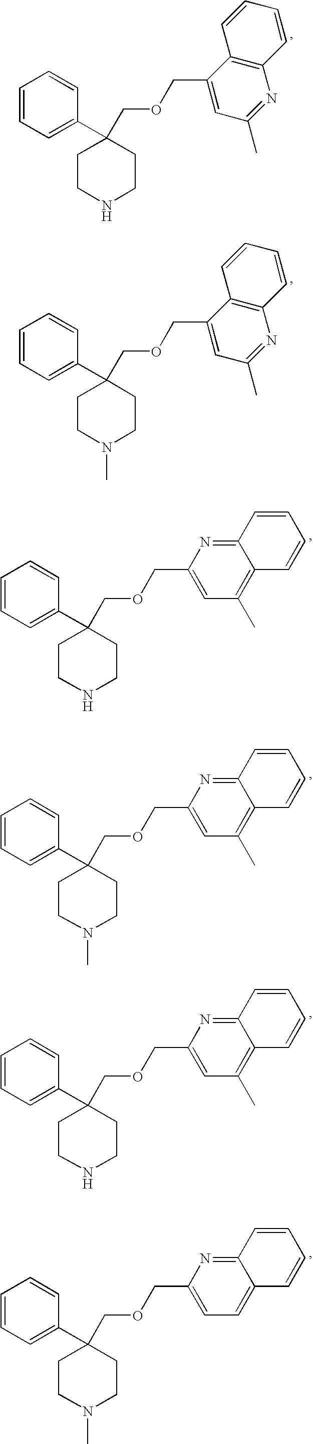 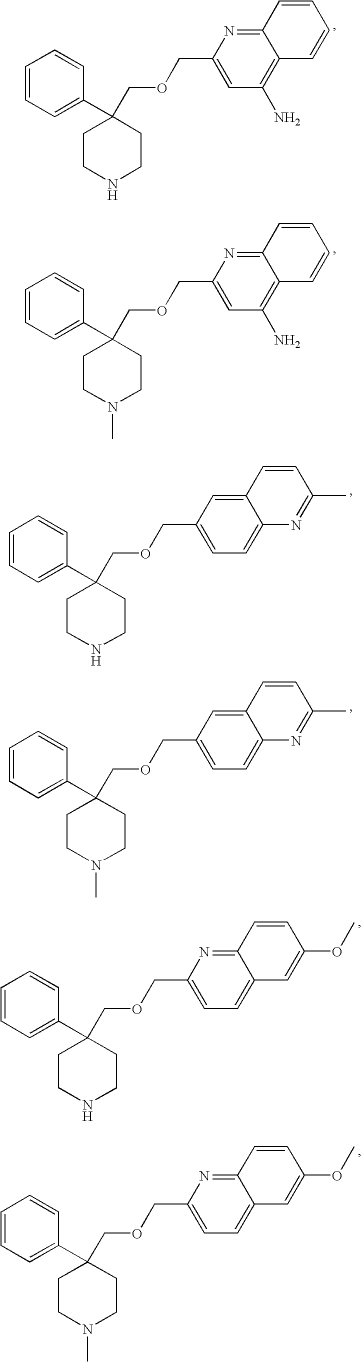 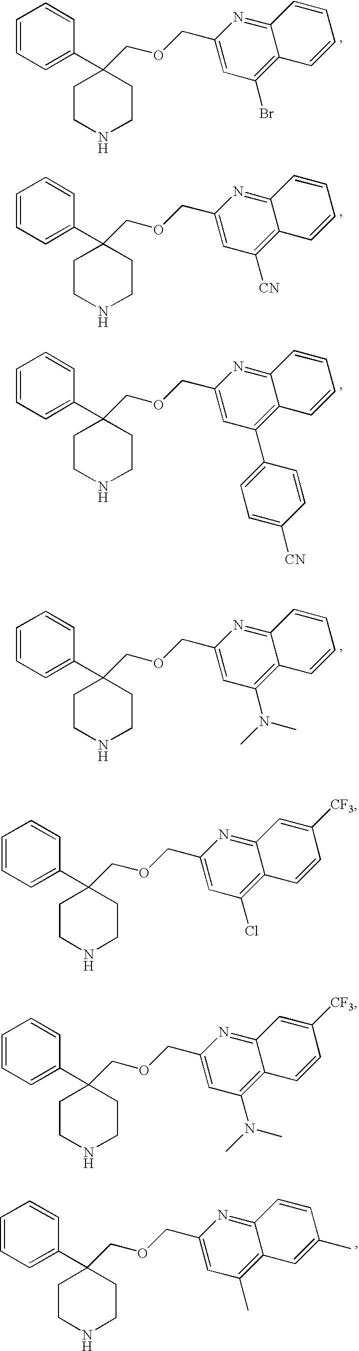 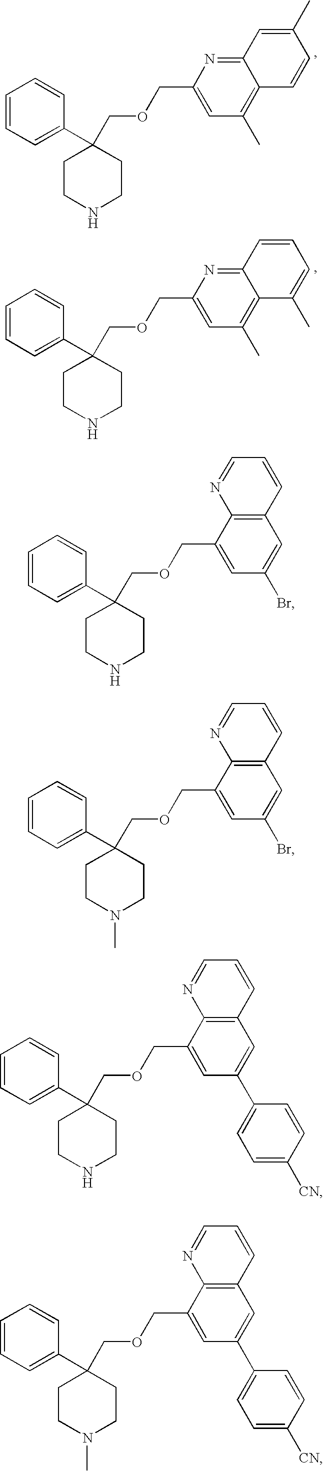 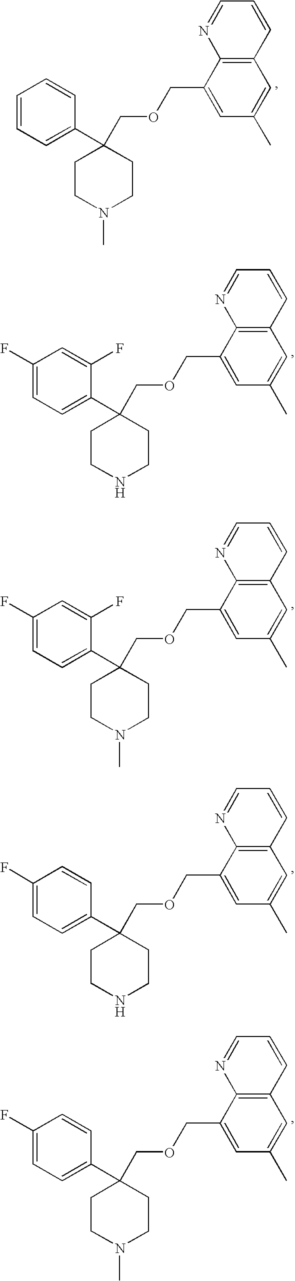 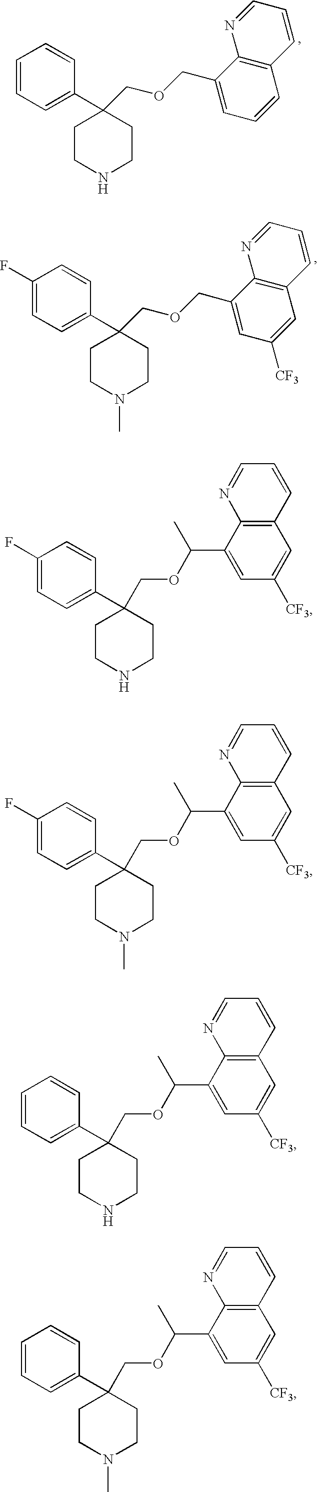 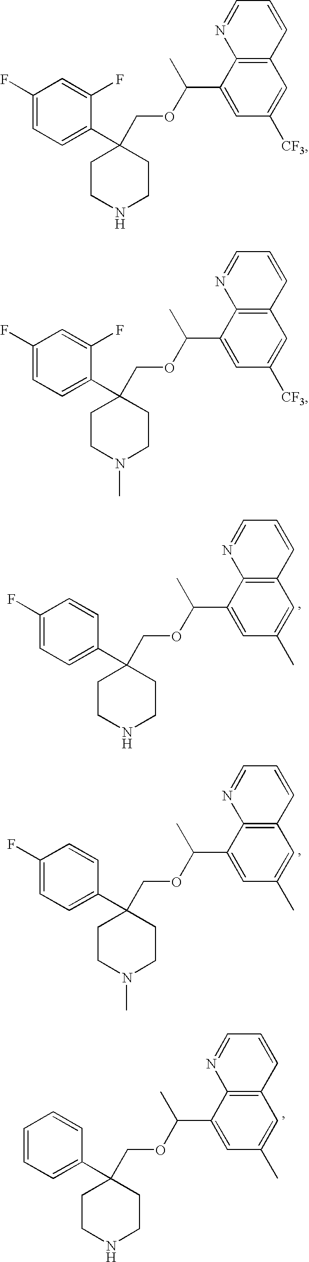 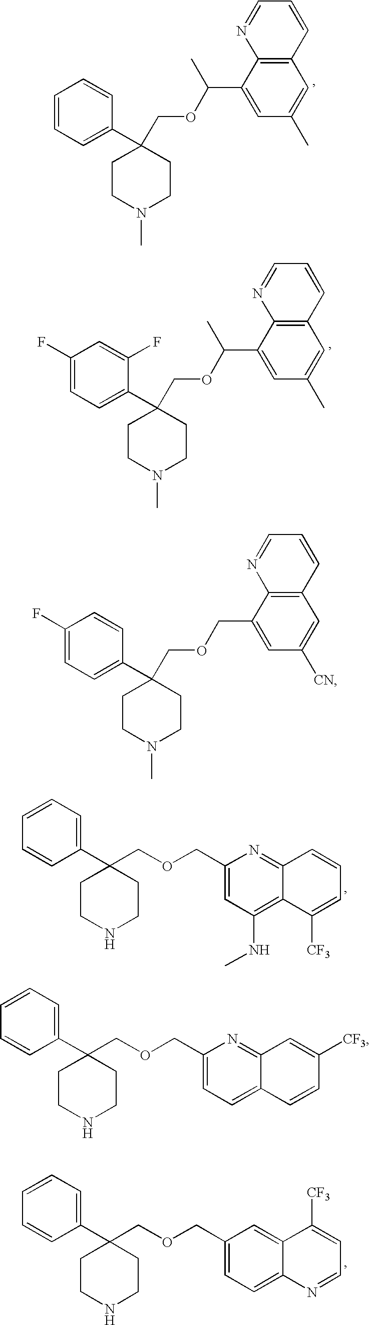 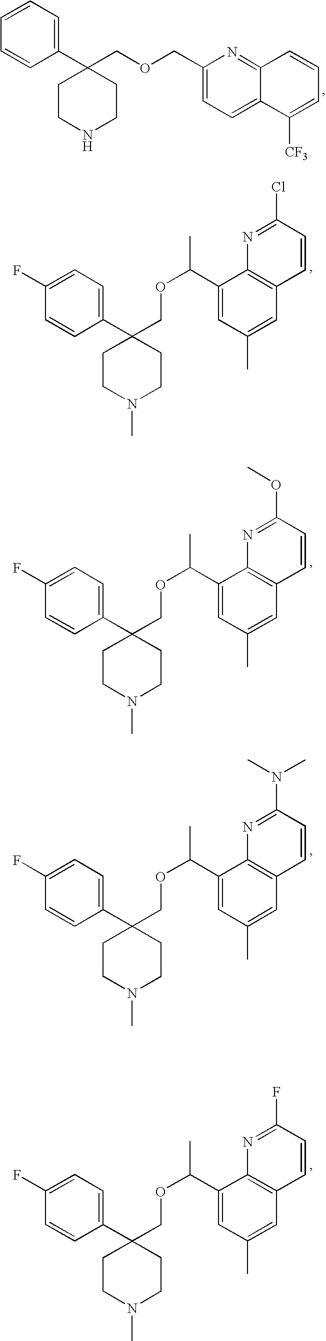 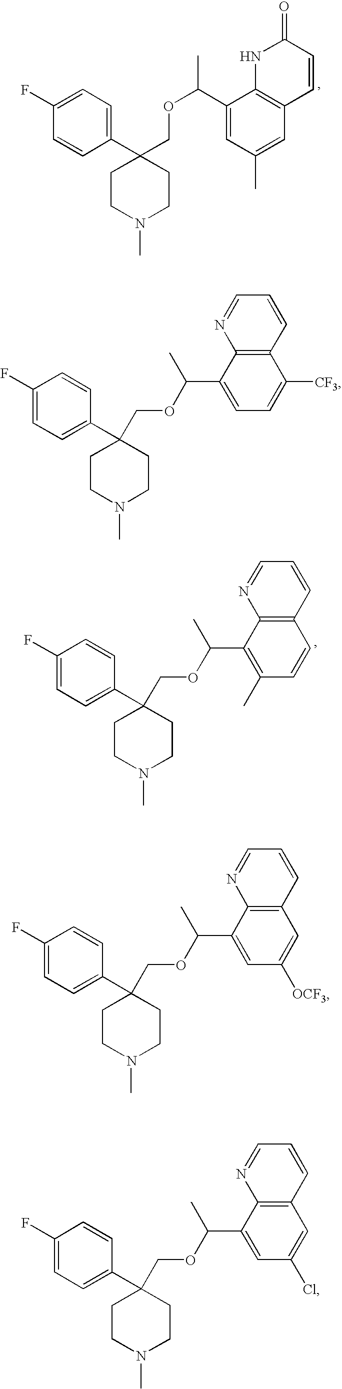 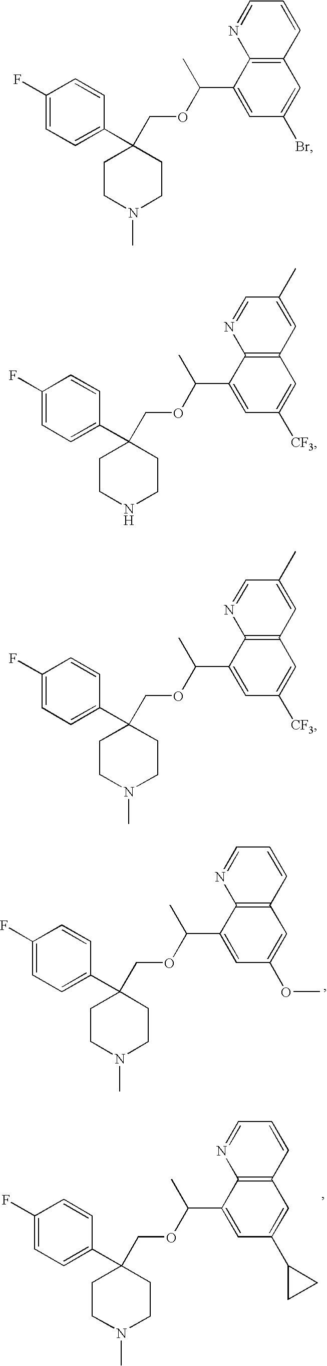 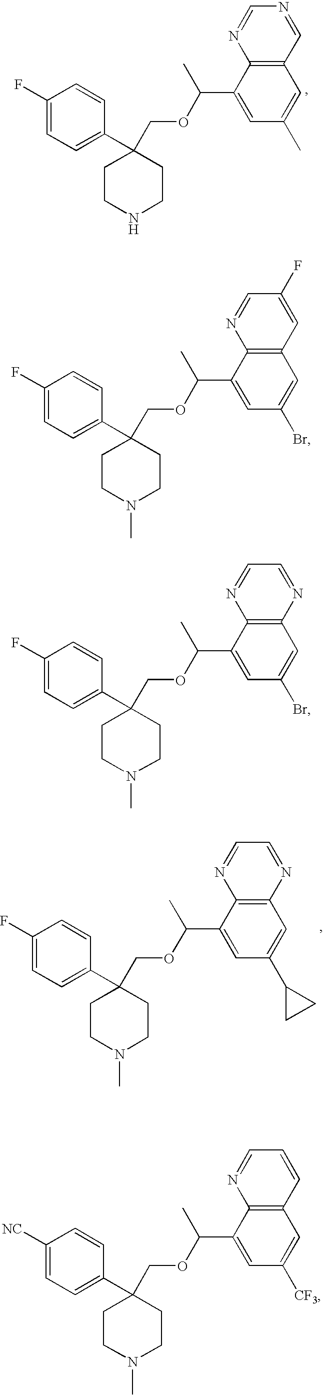 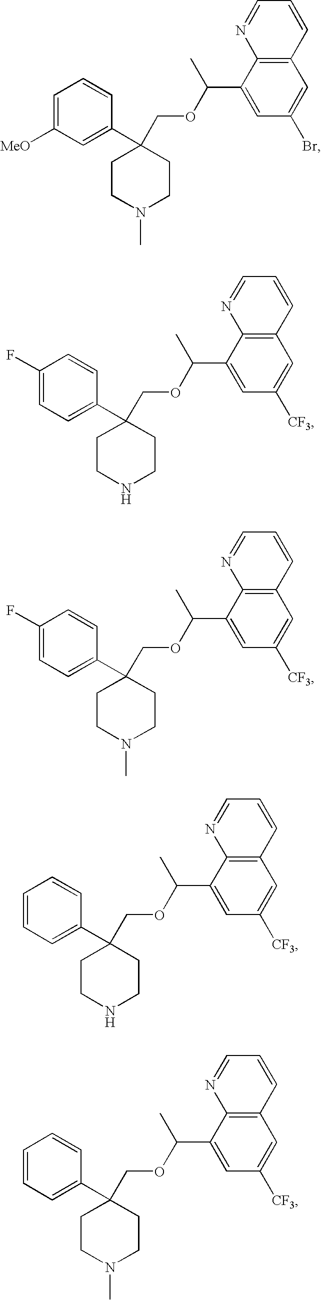 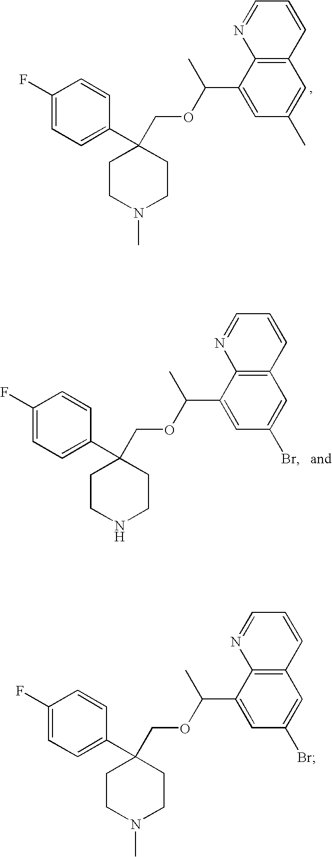
or a pharmaceutically acceptable salt thereof.

10. A composition comprising a pharmaceutically acceptable amount of a compound of claim 1 and a pharmaceutically acceptable carrier.

11. A method for treating a disorder associated with aberrant levels of tachykinins or serotonin comprising administering an effective amount of a compound of claim 1 to a patient afflicted with the disorder.

12. The method of claim 11 where the disorder is anxiety.

13. The method of claim 11 where the disorder is depression, obsessive compulsive disorder, bulimia, or panic disorder.
